(19) United States
(12) Patent Application Publication
(10) Pub. No.: US 2024/0100174 A1
(43) Pub. Date: Mar. 28, 2024

(54) PROTEIN-PEG INTERACTIONS THAT REDIRECT THE THERMAL UNFOLDING PATHWAY OF PEGYLATED HUMAN GALECTIN-3C

(71) Applicant: UNIVERSITY OF FLORIDA RESEARCH FOUNDATION, INCORPORATED, Gainesville, FL (US)

(72) Inventors: Matthew Eddy, Gainesville, FL (US); Michael Harris, Gainesville, FL (US); Amanda Pritzlaff, Gainesville, FL (US)

(21) Appl. No.: 18/236,078

(22) Filed: Aug. 21, 2023

Related U.S. Application Data
(60) Provisional application No. 63/399,437, filed on Aug. 19, 2022.

(51) Int. Cl. A61K 47/60; C07K 14/705

(52) U.S. Cl. CPC A61K 47/60; C07K 14/7056

(57) ABSTRACT

Conjugation of polymers to proteins, including biomedically-relevant PEGylation, is a promising approach to address a central challenge of biologics and biotech: the lack of protein stability in demanding non-native environments. Application of conjugation is hindered by the lack of atomic level understanding of protein-polymerinteractions, preventing design of conjugates with predicted properties. An integrative structural and biophysical approach was used to address this challenge using a polymer-modified carbohydrate recognition domain of human galectin-3 (Gal3C), a lectin essential for cellular adhesion and potential biologic. Modification with PEG and other polymers dramatically increased Gal3C thermal stability and redirected its unfolding pathway through forming a stable intermediate. Distinct polymer properties which increased protein thermal stability were revealed. Structural details of Gal3C-polymer conjugates revealed by NMR pointed to the important role of polymer localization. Residues local to the site of conjugation were perturbed by polymer conjugation and these perturbations remained localized over a wide temperature range. For PEGylated conjugates, replacing key lysine residues within the PEG-perturbed region altered the protein-PEG interface and thermal unfolding behavior, providing mechanistic insight into rational design of conjugates that will expand the benefits of polymer conjugation.

### GOVERNMENT FUNDING SUPPORT

This invention was made with government support under grant no. R35GM138291 awarded by The National Institutes of Health. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 4, 2023, is named “10457528US1_seq.xml” and is 28672 bytes in size. The sequence listing contained in this .XML file is part of the specification and is hereby incorporated by reference herein in its entirety.

### BACKGROUND

Biologics are highly effective, targeted treatments for a range of diseases, and constitute approximately 30% of drugs approved by the FDA over the past 5 years (1,2). Despite the promise of expanding on this success, biologics are inherently challenging to produce due to their complexity and frailty as compared with small molecules. One of the most promising approaches to overcoming these challenges is through chemical modification of biologics via covalent attachment of a polymer such as poly(ethylene glycol) (PEG) to reactive groups in protein side chains. PEGylation is one of the most widely used approaches in industrial and pharmaceutical applications (3-7). Since approval of the first PEGylated protein drug Adagen™ in 1990 nearly 30 FDA-approved PEGylated drugs have entered the clinic to treat a wide array of illnesses including numerous cancers (3).

Ideally, PEGylation should preserve or even enhance the activity of the protein drug while conferring improved robustness. The benefits of PEGylation to the conjugated protein include increased thermal stability , resistance to chemical denaturation, and improved functional activity (6,8-11). However, these benefits are not necessarily obtained with PEGylation at arbitrary positions in the protein sequence. PEGylation has been documented to also negatively affect protein properties, alter protein functions in undesired ways, or have no observable impact (12-15). Currently there exist no clear criteria for predicting the effects of PEGylation on protein properties. Thus, a grand challenge is designing protein-polymer conjugates a priori with predictable chemical properties.

Achieving this goal will require improved molecular models describing protein-polymer interactions with atomic level accuracy. Current molecular models mostly based on lower resolution methods proposed two different solution conformations of protein-PEG conjugates that differ in the degree of non-covalent interactions between PEG and the protein. In one model, the protein and covalently attached PEG are thought to exist as a ‘dumbbell’-shaped conjugate with minimal to no protein-PEG interactions (16-19). The second model describes a ‘shroud’ conformation where PEG forms more extensive interactions with the protein surface (20,21). Some experimental data and molecular dynamics simulations support a more nuanced view where the configuration of the protein-PEG conjugate depends on the protein size and the length and chemical structure of the conjugated polymer (22-24). The accuracy of these different models is difficult to assess, due in large part to the lack of experimental data that provide atomic level information on the structure and conformational dynamics of the protein-polymer conjugate.

Galectin-3 has well-documented roles in multiple disease areas, including cancer (26-28) where it is critical for angiogenesis and metastasis (29). Galectin-3 also forms the lubricating layer of articular cartilage (30) and plays important roles in endometriosis development and is a target for endometriosis treatments (31). Gal3C, the naturally-occurring carbohydrate recognition domain only form of the galectin-3 protein, has been shown to be a potential biologic useful in cancer treatment, inhibiting tumor growth in breast cancer animal models (32) and enhancing the activity of anti-cancer compounds (33).

### SUMMARY

This invention relates to compositions containing the Gal3C protein which may have one or more amino acids substituted for the natural residue and which also have a synthetic polymer covalently attached through a single amino acid group. Specifically, these compositions as prepared are more temperature resistant than the unmodified Gal3C protein through means of a polymer-dependent redirection of the protein unfolding pathway. The stability of these conjugates allowed for thorough characterization of elevated-temperature protein behavior at the molecular scale. This disclosure contains aspects of methods used to prepare these compositions as well as the thorough characterization of the discretely prepared conjugates. Specifically, characterization focused on realizing residue-specific perturbations of the protein structure and dynamics caused by the attached polymer over a range of temperatures. We recorded NMR spectroscopic data with aqueous solutions containing PEG conjugated to the carbohydrate recognition domain of human galectin-3 (Gal3C). Global secondary structure, thermal unfolding transitions, and quantitative melting temperatures were obtained using circular dichroism (CD) spectroscopy. Protein and conjugate function was assessed using equilibrium binding intrinsic tryptophan fluorescence assays.

### BRIEF DESCRIPTION OF THE DRAWINGS

FIG. 1A shows the crystal structure of Gal3C (PDB ID 4R9A) in a ribbon representation in two orthogonal orientations (64). Position T243, which was replaced with a cysteine for chemical modification with PEG, is represented by a red sphere in both images. FIG. 1B shows the grafting-to conjugation scheme for PEGylation of Gal3C[T243C] using a thiol-Michael addition. FIG. 1C shows the equilibrium binding of LacNac to Gal3C[T243C] (black) and Gal3C[T243C]-PEG (red) measured by intrinsic tryptophan fluorescence. FIG. 1D presents a superposition of normalized circular dichroism (CD) plots of Gal3C[T243C] measured from 30° C. (region ‘1’) to 90° C. (region ‘2’). The arrow and ‘T’ indicate an increase in temperature for individual spectra plotted from region ‘1’ to region ‘2’. FIG. 1E presents a superposition of normalized CD plots of Gal3C[T243C]-PEG measured from 30° C. to 90° C. FIG. 1F shows the thermal unfolding of Gal3C[T243C] (black) and Gal3C[T243C]-PEG (red) monitored by CD at 220 nm.

FIG. 2A shows the superposition of [¹⁵N, ¹H]-HSQC spectra of [u-¹⁵N]-Gal3C[T243C] (blue) and [u-¹⁵N]-Gal3C[T243C]-PEG (orange) measured at 30° C. FIG. 2B presents expanded views from FIG. 2A selected to show specific amide signals where significant changes in chemical shift or line widths were observed between Gal3C[T243C] and Gal3C[T243C]-PEG. FIG. 1C is a surface representation of the crystal structure of Gal3C (PDB ID 4R9C). FIG. 2D is a histogram of the chemical shift perturbations (blue bars) and line broadening (orange bars) observed for Gal3C[T243C]-PEG relative to the unconjugated protein as a function of the residue position.

FIG. 3A shows the rotational correlation times (τ_{c}) of Gal3C[T243C] (blue) and Gal3C[T243C]-PEG (orange) at 30° C. determined from ¹⁵N relaxation data shown in FIG. 3B. FIG. 3B shows the global ¹⁵N longitudinal and transverse relaxation times (T₁ and T₂, respectively) of amide groups in the structured core of Gal3C[T243C] and Gal3C[T243C]-PEG.

FIG. 4A shows the solvent accessibility of amide groups of Gal3C[T243C]-PEG determined from HDX experiments (in purple) mapped onto the crystal structure of Gal3C (PDB ID 4R9C). The residues perturbed by PEGylation of the Gal3C protein are shown in grey space filling dots for comparison. FIG. 4B shows perturbations to amide groups at orange colored residues after heating Gal3C[T243C]-PEG to the first transition temperature of 62° C. and then cooling back down to 30° C. FIG. 4C is a histogram of the chemical shift perturbations (blue bars) and line broadening (orange bars) observed at 30° C. for Gal3C[T243C]-PEG after heating to 62° C. relative to the native protein conjugate.

FIG. 5A (Gal3C[T243C]-PEG), FIG. 5B (Gal3C[T243C,K139I]-PEG), and FIG. 5C (Gal3C[T243C,K139I, K196I]-PEG) are ribbon representations of Gal3C with PEG-perturbed residues colored in green for each variant. FIG. 5D, FIG. 5E, and FIG. 5F present data on the thermal unfolding of Gal3C[T243C,K139I] and the corresponding PEGylated variant. FIG. 5G, FIG. 5H, and FIG. 5I present data on the thermal unfolding of Gal3C[T243C,K139I,K196I] and the corresponding PEGylated variant. Thermal unfolding of variants was monitored by variable temperature CD.

FIG. 6 is a size exclusion chromatogram from a representative sample preparation of Gal3C[T243C]-PEG.

FIG.7 is a representative MALDI-TOF spectrum of a Gal3C[T243C]-PEG sample used for biophysical experiments, showing a single peak near the expected m/z ratio (22.3 kDa).

FIG. 8 is a representative size exclusion chromatogram of the products of the Gal3C PEGylation reaction after removal of excess PEG-maleimide.

FIG. 9 shows equilibrium binding of lactose to Gal3C (black squares), Gal3C[T243C] (red circles) and Gal3C[T243C]-PEG (blue triangles) measured by intrinsic tryptophan fluorescence.

FIG. 10 shows equilibrium binding of LacNac to Gal3C (black squares) measured by intrinsic tryptophan fluorescence.

FIG. 11 shows superposition of normalized circular dichroism (CD) vs. wavelength plots of Gal3C (black squares), Gal3C[T243C] (red circles) and Gal3C[T243C]-PEG (blue triangles) measured at 30° C.

FIG. 12 shows superposition of normalized circular dichroism (CD) plots of Gal3C measured from 30° C. (region ‘1’) to 90° C. (region ‘2’).

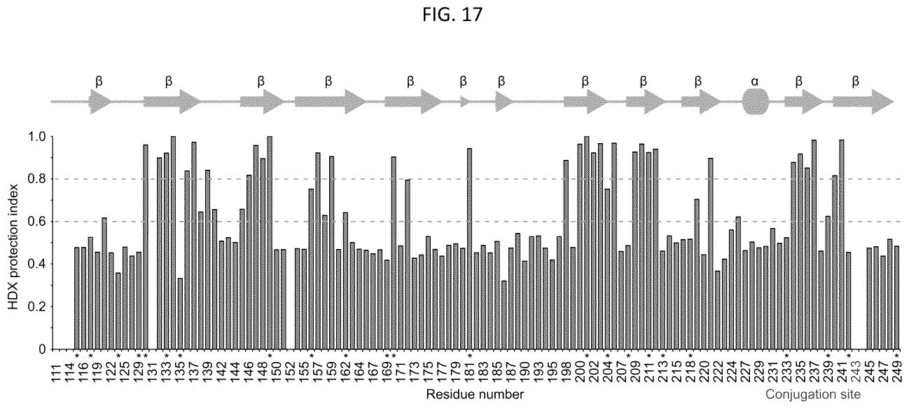

FIG. 13 shows the thermal unfolding of Gal3C (black squares) monitored by normalized CD intensity at 220 nm.

FIG. 14 shows thermal unfolding of Gal3C[T243C] (20 μm) in the presence (black squares) and absence (red circles) of 22 μM free PEG-OH measured by normalized CD intensity at 220 nm.

FIG. 15 shows one-dimensional cross sections from [¹⁵N, ¹H]-HSQC spectra of [u-¹⁵N]-Gal3C[T243C] (blue) and [u-¹⁵N]-Gal3C[T243C]-PEG (orange).

FIG. 16A presents a superposition of ¹⁵N-edited 1D ¹H spectra of [u-¹⁵N]-Gal3C[T243C] (blue) and [u-¹⁵N]-Gal3C[T243C]-PEG (orange). FIG. 16B shows the correlation of rotational correlation times (τ_{c}) with molar masses plotted as blue circles for 20 well-characterized globular proteins from literature data.

FIG. 17 is a histogram of the relative resistance of Gal3C[T243C]-PEG amide protons to exchange with deuterium in a solution containing 73.5% ²H₂O, indicated as the “HDX protection index”.

FIG. 18A is a histogram plot of chemical shift perturbations (CSP) observed in [¹⁵N, ¹H]-HSQC spectra of Gal3C[T243C]-PEG relative to the unconjugated protein as measured at 40° C. (yellow), 50° C. (orange) and 55° C. (red). FIG. 18B is a histogram plot of observed line broadening in [¹⁵N, ¹H]-HSQC spectra of Gal3C[T243C]-PEG relative to the unconjugated protein as measured at 40° C. (yellow), 50° C. (orange) and or 55° C. (red). FIG. 18C is a histogram plot of differences in chemical shift perturbations between [¹⁵N, ¹H]-HSQC spectra of Gal3C[T243C]-PEG relative to Gal3C[T243C] compared between spectra measured at 55 ° C. and 30° C.

FIG. 19 shows the superposition of normalized circular dichroism (CD) plots recorded during the preparation of the intermediate state of Gal3C[T243C]-PEG by heating from 30° C. to 62° C.

FIGS. 20A-B show the superposition of [¹⁵N, ¹H]-HSQC spectra measured at 30° C. with freshly prepared [u-¹⁵N]-Gal3C[T243C]-PEG (blue) and for the same sample heated to 62° C. for 3 minutes and then cooled back down to 30° C. (orange). FIG. 20A shows the overall [¹⁵N, ¹H]-HSQC spectra; FIG. 20B shows a set of expanded views presenting NMR signals that were perturbed upon heating the sample.

FIGS. 21A-B show the superposition of [¹⁵N, ¹H]-HSQC spectra measured at 30° C. with unconjugated and PEGylated [u-¹⁵N]-Gal3C[T243C, K139I] (FIG. 21A) and [u-¹⁵N]-Gal3C[T243C, K139I, K196I] (FIG. 21B).

FIGS. 22A-22D show histograms of the chemical shift perturbations (blue bars) and line broadening (orange bars) observed for the residues of PEGylated Gal3C variants relative to their unconjugated counterparts. FIG. 22A: Gal3C[T243C]-PEG relative to Gal3C[T243C] as shown in FIG. 2D; FIG. 22B: Gal3C[T243C,K139I]-PEG relative to Gal3C[T243C,K139I]; FIG. 22C: Gal3C[T243C,K139I,K196I]-PEG relative to Gal3C[T243C,K139I,K196I].

FIG. 23 shows a ¹H NMR spectrum of mPEG-maleimide (PEG) in CDCl₃ measured at room temperature and 400 MHz ¹H precession frequency. Relative signal integrals and assignments are shown.

FIG. 24 is a histogram showing differences in observed line width of NMR signals observed in HSQC spectra of [u-¹⁵N] Gal3C[T243C] and [u-¹⁵N] Gal3C[T243C]-PEG at temperatures 40, 50, and 55° C.

FIG. 25A is a graph showing thermal melt profiles measured via circular dichroism of Gal3C[T243C,K139I] and Gal3C[T243C,K139I]-PEG. FIG. 25B is a graph showing thermal melt profiles measured via circular dichroism of Gal3C[T243C,K139I, K196I] and Gal3C[T243C,K139I,K196I]-PEG.

FIG. 26 is a graph showing thermal melt profiles measured via circular dichroism of Gal3C[T243C,K139I, K196I, K199I].

FIGS. 27A-27C Properties of polymers used to generate conjugates with Gal3C[T243C] and general conjugation scheme. (FIG. 27A) Grafting-to conjugation scheme for generating Gal3C[T243C]-polymer conjugates using RAFT-generated polymers. (FIG. 27B) Chemical structures of monomers dimethylacrylamide (DMA) and oligoethylene glycol methacrylate (OEGMA) of number-averaged molecular weights (Mₙ's) of 300 and 500 ethylene glycol (EG) derived poly(ethylene glycol) (PEG) is shown for comparison. (FIG. 27C) Final Mₙ values of dispersities (Ð's) of polymers conjugated to Gal3C[T243C] as obtained by SEC.

FIGS. 28A-28E Preparation and equilibrium binding affinities of Gal3C[T243C]-P(OEGMA⁵⁰⁰)₂₁ and Gal3C[T243C]-PDMA₆₁ conjugates compared to unmodified Gal3C[T243C]. (FIG. 28A) Reference chromatogram of unmodified Gal3C[T243C]. (FIG. 28B and FIG. 28C) SEC purification of (B) Gal3C[T243C]-P(OEGMA⁵⁰⁰)₂₁ and (C) Gal3C[T243C]-PDMA₆₁. (FIG. 28D and FIG. 28E) SDS-PAGE of representative conjugate samples for (FIG. 28D) Gal3C[T243C]-P(OEGMA⁵⁰⁰)₂₁ and (FIG. 28E) Gal3C[T243C]-PDMA₆₁. For both gels, the left lane is an annotated molecular weight marker. (FIG. 28F) Equilibrium binding of LacNac to Gal3C[T243C]-P(OEGMA⁵⁰⁰)₂₁, Gal3C[T243C]-PDMA₆₁ and Gal3C[T243C] measured with intrinsic tryptophan fluorescence. Dissociation constants (K_{D}'s) for LacNac are reported in the figure legend as an average of three replicates±standard deviation. A single replicate with a K_{D} closest to the average is plotted for each construct.

FIGS. 29A-29F Effects of conjugation with 5.4 kDa PEG, PDMA₆₁, and P(OEGMA⁵⁰⁰)₂₁ on the backbone structure and dynamics of [U-¹⁵N]-Gal3C[T243C] as observed by chemical shift perturbations (CSPs) and line broadenings between [¹⁵N, ¹H]-HSQC NMR spectra of Gal3C[T243C] and conjugates. (FIG. 29A-C) Histograms reporting CSPs and line broadening of backbone amide signals for Gal3C[T243C] conjugated with (FIG. 29A) PEG, (FIG. 29B) PDMA₆₁, and (FIG. 29C) P(OEGMA⁵⁰⁰)₂₁. The “*” indicates residues with ambiguous assignments. The green “#” indicates backbone amides broadened beyond detection or unassignable. The conjugation site, C243, is indicated by the red line. Areas of significant perturbation due to PEGylation at C243, as observed in a previous study of Gal3C[T243C], are outlined with gray dashed boxes on each plot. (FIG. 29D-F) Mapping chemical shift perturbations and line broadenings on the structure of Gal3C[T243C] for the conjugates with (FIG. 29D) PEG, (FIG. 29E) PDMA₆₁, and (FIG. 29F) P(OEGMA⁵⁰⁰)₂₁. Gal3C[T243C] (PDB ID 4R9A) is shown in space-filling representation with residues colored according to the lower right legend. Panels A and D are adapted with permission from ref (33). Copyright 2022 John Wiley & Sons.

FIGS. 30A-30J Preparation and thermal melting profiles of Gal3C[T243C]-PDMA₁₀, Gal3C[T243C]-PDMA₃₉, and Gal3C[T243C]-PDMA₆₁. (FIG. 30A) Reference chromatogram of unmodified Gal3C[T243C]. (FIG. 30B-D) SEC chromatograms of (FIG. 30B) Gal3C[T243C]-PDMA₁₀, (FIG. 30C) Gal3C[T243C]-PDMA₃₉, and (FIG. 30D) Gal3C[T243C]-PDMA₆₁ with exact fractions selected for characterization labeled a-e, A-E, and 1-4, respectively. All fractions were 330 μL. (FIG. 30E-G) SDS-PAGE gels of select conjugate fractions for I Gal3C[T243C]-PDMA₁₀, (FIG. 30F) Gal3C[T243C]-PDMA₃₉, and (FIG. 30G) Gal3C[T243C]-PDMA₆₁. (FIG. 30H-J) Thermal melting plots monitored by single-wavelength circular dichroism of (FIG. 30H) Gal3C[T243C]-PDMA₁₀ fractions b-d, (FIG. 30I) Gal3C[T243C]-PDMA₃₉ fractions C-E, and (FIG. 30J) Gal3C[T243C]-PDMA₆₁ fractions 1-3. The thermal melting profile of unmodified Gal3C[T243C] is superimposed in panels H-J.

FIGS. 31A-31G Preparation and thermal melting of Gal3C[T243C]-P(OEGMA³⁰⁰)₂₀ and Gal3C[T243C]-P(OEGMA⁵⁰⁰)₂₁. (FIG. 31A) Reference chromatogram of unmodified Gal3C[T243C]. (FIG. 31B) SEC purification of Gal3C[T243C]-P(OEGMA³⁰⁰)₂₀ with exact fractions selected for characterization labeled 1-6. All fractions were 330 μL. (FIG. 31C) SEC purification of Gal3C[T243C]-P(OEGMA⁵⁰⁰)₂₁ with exact fractions selected for characterization labeled A-D. (FIG. 31D and FIG. 31E) SDS-PAGE of select conjugate fractions for Gal3C[T243C]-P(OEGMA³⁰⁰)₂₀ and Gal3C[T243C]-P(OEGMA⁵⁰⁰)₂₁, respectively. The unmodified Gal3C[T243C] lane in (FIG. 31D) features both monomeric (17 kDa) and the occasionally observed dimer (34 kDa) in sample. (FIG. 31F) Single-wavelength thermal melting plots of Gal3C[T243C]-P(OEGMA³⁰⁰)₂₀ fractions 2-4 as compared to unmodified Gal3C[T243C]. (FIG. 31G) Single-wavelength thermal melting plots of Gal3C[T243C]-P(OEGMA⁵⁰⁰)₂₁ fractions A-D as compared to unmodified Gal3C[T243C].

FIGS. 32A-32C Two-step scheme for functionalization of trithiocarbonate end-group polymers with bis-maleimide linker (1). Each step was followed by desalting in PBS. FIG. 32A teaches a first scheme of functionalization. FIG. 32B teaches a second scheme for producing PDMA-SH or POEGMA-SH. FIG. 32C shows a scheme for generating PDMA-Mal or POEGMA-Mal.

FIGS. 33A-33E Size exclusion chromatograms of polymers obtained using gel permeation chromatography before (solid black lines) and after (red dashed lines) end-group functionalization with bismaleimide linker. FIG. 33A shows the chromatogram for PDMA10, FIG. 33B shows the chromatogram for PDMA39, FIG. 33C shows the chromatogram for PDMA61; FIG. 33D shows the chromatogram for P(OEGMA⁵⁰⁰)21 and FIG. 33E shows the chromatogram for P(OEGMA³⁰⁰)20 in DMAC solvent.

FIG. 34 Superimposed size exclusion chromatograms of P(OEGMA⁵⁰⁰)21 (solid black line), PDMA61 (blue dash-dotted line) and reference 5,000 g/mol PEG-maleimide (red dashed line) in DMAC solvent.

FIGS. 35A-35B ¹H NMR spectra of POEGMA polymers before and after functionalization with bis- maleimide (1), (FIG. 35A) P(OEGMA⁵⁰⁰)21, and (FIG. 35B) P(OEGMA³⁰⁰)20. Bis-maleimide linker (1) is provided as the top spectrum in A and B in blue. End-group functionalized polymers are shown in the middle spectra in green. Unmodified polymers are shown in the bottom red spectra. Inset spectra show the maleimide region of the spectra from δ6.5-6.95 ppm. Broad peaks labeled with an asterisk (*) in the green spectra in A and B are impurity peaks related to isolating polymer from buffer prior to dissolving in NMR solvent. Spectra were recorded on a 500 MHz Varian instrument in CDCl3 (see methods Example 8).

FIGS. 36A-36C ¹H NMR spectra of PDMA polymers before and after functionalization with bis-maleimide (1), (FIG. 36A) PDMA61, (FIG. 36B) PDMA39, and (FIG. 36C) PDMA10. Bis-maleimide linker (1) is shown as the top spectrum in blue. End-group functionalized polymers are shown in the middle spectra in green. Unmodified polymers are shown in the bottom red spectra. Inset spectra show the maleimide region of the spectra from 6.0-6.8 ppm. PDMA10-mal and PDMA39-mal (green spectra FIG. 36B and FIG. 36C) exhibited polydispersity in SEC chromatograms (FIG. 33), including possible polymers dimerized by a single bis-maleimide, and therefore spectra are provided without further assignment. Spectra were recorded on a 500 MHz Varian instrument in D20 for PDMA39 and in CDCl3 for all other samples.

FIGS. 37A-37D UV-Vis spectra of polymers before (blue traces) and after (black dashed traces) end-group cleavage with hydrazine. Samples were prepared by diluting 20-30× prior to acquisition and each polymer is prepared at the same dilution. FIG. 37A shows the spectra for PDMA₃₉ and PDMA₃₉-SH, FIG. 37B shows the spectra for PDMA₆₁ and PDMA₆₁-SH, FIG. 37C shows the spectra for POEGMA³⁰⁰ and POEGMA³⁰⁰-SH, FIG. 37D shows the spectra for POEGMA⁵⁰⁰ and POEGMA⁵⁰⁰-SH

FIG. 38 ¹H NMR spectrum measured in CDCl3 measured on a Varian 500 MHZ instrument with the bis-maleimide linker (1) used to functionalize polymer thiol end groups.

FIGS. 39A-39D Determination of potential side products from the reaction between Gal3C[T243C] and 10 equivalents of 5k PEG-maleimide as monitored by SEC and SDS-PAGE. FIG. 39A) Reference Gal3C[T243C] SEC chromatogram. FIG. 39B-C) Reaction time points of 1, 4.5, or 12 h as shown in (FIG. 39B) superimposed chromatograms and (FIG. 39C) crude aliquots analyzed by SD-PAGE. (FIG. 39D) MALDI-TOF spectrum of 4.5 h sample time point showing masses (m/z) of 22.4 kDa and 44.7 kDa, at expected masses of Gal3C[T243C]-PEG conjugate and conjugate dimer, respectively. Spectrum was acquired in linear mode 20-50 kDa with a 10 mg/mL sinapinic acid matrix in 1:1 ACN:water (0.1% TFA).

FIGS. 40A-40C SEC-purified conjugates before (black traces) and after (red dash traces) reduction with DTT. (FIG. 40A) Reference chromatogram of Gal3C[T243C] with 10 mM DTT. (FIG. 40B) Gal3C[T243C]-PDMA61 before and after DTT reduction. (FIG. 40C). Gal3C[T243C]-P(OEGMA⁵⁰⁰)21 before and after DTT reduction. Regions on (FIG. 40B) and (FIG. 40C) marked “Gal3C[T243C]/free polymer” are labeled according to Gal3C[T243C] elution times shown in (FIG. 40A) and elution time of free polymers (not shown).

FIG. 41 Superposition of [¹⁵N, ¹H]-HSQC spectra measured on a Bruker 800 MHz instrument at 30° C. of Gal3C[T243C] (blue) and Gal3C[T243C] conjugated to PEG (orange), as adapted with permission from reference 4 Example 8.

FIG. 42 Superposition of [¹⁵N, ¹H]-HSQC spectra measured on a Bruker 800 MHz instrument at 30° C. of Gal3C[T243C] and Gal3C[T243C] conjugated to different polymers. The spectra of [u-¹⁵N]-Gal3C[T243C] (green) and [u-¹⁵N]-Gal3C[T243C]-PEG (red) is superimposed with the spectrum of [u-¹⁵N]-Gal3C[T243C]-PDMA (blue) or [u-¹⁵N]-Gal3C[T243C]-P(OEGMA⁵⁰⁰)21 (purple).

FIGS. 43A-43F Superposition of full CD vs. wavelength thermal melting plots recorded from 30 to 90° C., which were used to generate thermal melting curves shown in FIGS. 4 and 5. (FIG. 43A) Gal3C[T243C] spectra. (FIG. 43B-F) Melting plots Gal3C[T243C] conjugated with (FIG. 43B) P(OEGMA³⁰⁰)₂₀, (FIG. 43C) P(OEGMA⁵⁰⁰)₂₁, (FIG. 43D) PDMA₁₀, (FIG. 43E) PDMA39, and (FIG. 43F) PDMA₆₁.

FIG. 44 [¹⁵N, ¹H]-HSQC spectrum of [u-¹⁵N]-Gal3C[T243C]-P(OEGMA⁵⁰⁰)21 measured at 55° C. on a Bruker 800 MHz instrument.

### DETAILED DESCRIPTION

Embodiments described herein pertain to polymer conjugation of Gal3C or variants thereof that result in a Gal3C-polymer conjugate which has a thermal unfolding intermediate and possesses increased thermal stability while maintaining proper formation and biological activity at physiological temperatures. In certain embodiments, disclosed is an agent comprising a sequence according to SEQ ID NO:11, or a sequence comprising at least 90%, 92%, 95%, or 98% therewith, that includes at least one substituted cysteine residue substituted in place of rationally-selected residue, wherein the sequence is conjugated to a polymer at the at least one substituted cysteine residue. In specific embodiments, the polymer is PEG, PDMA, or POEGMA. In an exemplified embodiment, the at least one substituted cysteine residue replaces threonine at position 243 (T243C). The polymer and protein sequence may be linked by a succinimide molecule as the result of a thiol-Michael addition between polymeric thiol and maleimide linker.

According to other embodiments, disclosed is a method for treating a subject with cancer comprising administering a therapeutically effective amount of a composition comprising an polymerized Gal3C agent as described herein. In certain examples, the polymer is PEG, PDMA or POEGMA. In a specific embodiment, the agent comprises Gal3C[T243C] conjugated to a PEG polymer.

Other embodiments pertain to a method of improving the pharmacological properties of a protein drug compound. The method involves a) obtaining a Gal3C sequence or variant thereof comprising at least 90%, 92%, 95% or 98% sequence identity therewith, wherein the Gal3C sequence or variant thereof comprises at least one substituted cysteine residue in place of a rationally-selected residue; and b) conjugating a polymer to the Gal3 sequence or variant thereof to the at least one substituted cysteine residue. The conjugating step may comprise combining a polymer comprising a maleimide molecule covalently bound thereto, wherein the maleimide molecule reacts with a thiol group of the at least one substituted cysteine residue resulting in the polymer being linked to a sulfur of the at least one substituted cysteine residue via a succinimide molecule. In specific examples, the polymer covalently bound to a maleimide molecule comprises at least one of the following:

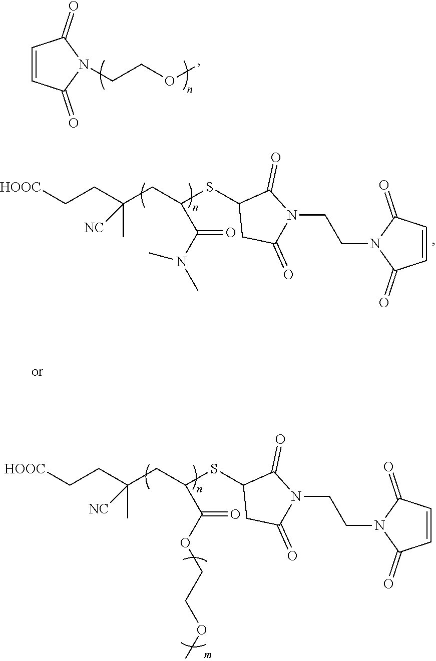

### Overview

High resolution NMR data of the unconjugated and conjugated proteins provided an atomic level view into the effects of PEGylation on the structure of PEGylated Gal3C. Using variable temperature circular dichroism (CD) spectroscopy, PEGylation was demonstrated to redirect the unfolding pathway of Gal3C. Gal3C PEGylation resulted in the formation of a stable intermediate conformation upon heating, which had been previously proposed to account for the effects of PEGylation on the stability of other proteins but not experimentally observed (13,34). Local Gal3C-polymer interactions observed by NMR correlated with a redirection of the thermal unfolding pathway, which could be influenced by replacing specific charged residues with more hydrophobic amino acids. Surprisingly, PEGylation of these Gal3C variants still provided improved thermal stability despite altered interactions. Together these data provide a path toward the rational design of protein-polymer conjugates with predicted properties. In certain embodiments, the protein Galectin-3C (Gal3C) is modified with cysteine at the 243 position (Gal3C[T243]). PEG (121)monomethylether was converted to the amine using MsCl and ammonium hydroxide to give the PEG (121) amine that was reacted with maleic anhydride to PEG (121) maleimide, which was subsequently reacted in a Michael addition reaction with the SH of the cysteine to give the conjugated Gal3C[T243C]-PEG.

Note that the description and examples refer to Gal3C, but the invention also is contemplated for use with other proteins, such as industrial enzymes benefitting from increased thermal stability and/or melting temperatures and PEGylated therapeutic.

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about” means plus or minus 20 percent of the recited value, so that, for example, “about 0.125” means 0.125±0.025, and “about 1.0” means 1.0±0.2.

As used herein, the term “cancer” refers to a neoplasm or tumor resulting from abnormal uncontrolled growth of cells. The term “cancer” encompasses a disease involving both pre-malignant and malignant cancer cells. In some embodiments, cancer refers to a localized overgrowth of cells that has not spread to other parts of a subject, i.e., a benign tumor. In other embodiments, cancer refers to a malignant tumor, which has invaded and destroyed neighboring body structures and spread to distant sites. In yet other embodiments, the cancer is associated with a specific cancer antigen.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”. “having”, “has”, “with”, or variants thereof are used in either the detailed description and/or the claims, such terms are intended to be inclusive in a manner similar to the term “comprising.”

As used herein, the term “percentage of sequence identity” or “percent sequence identity” refers to the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. A sequence that is identical at every position in comparison to a reference sequence is said to be 100% identical to the reference sequence, and vice-versa. The percent identity of two nucleotide sequences may be determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. Optimal alignment to compare two or more sequences may be performed using local or global alignment through a variety of available computer programs. The algorithm of Smith T. F. and Waterman M. S. (1981), Identification of common molecular subsequences [symbol] J. Mol. Biol. 147(1):195-,PubMed: 7265238 [symbol] DOI: 10.1016/0022-2836(81)90087-5 is a suitable local alignment strategy and is utilized by tools such as EMBOSS Water (https://www.ebi.ac.uk/Tools/psa/emboss_water/). The algorithm of Needleman S. B. and Wunsch C. D. (1970), A general method applicable to the search for similarities in the amino acid sequence of two proteins, J. Mol. Biol. 48(3):443-53,PubMed: 5420325,DOI: 10.1016/0022-2836(70)90057-4 is a suitable global alignment strategy and is utilized by such tools as EMBOSS Needle (https://www.ebi.ac.uk/Tools/psa/emboss_needle/). Depending on the sequences to be compared and the relevant parameters, a local or global alignment strategy may be more likely to find an optimal alignment, but both strategies may be utilized to confirm the optimal alignment giving the most accurate percent identity.

The terms “treat” “treating” or “treatment of” as used herein refers to providing any type of medical management to a subject. Treating includes, but is not limited to, administering a composition comprising one or more active agents to a subject using any known method. for purposes such as curing, reversing, alleviating, reducing the severity of, inhibiting the progression of, or reducing the likelihood of a disease, disorder, or condition or one or more symptoms or manifestations of a disease, disorder or condition. The administration of the drug can be oral, nasal, parental, topical, ophthalmic, or transdermal administration or delivery in the form of solid, semi-solid, lyophilized powder, or liquid dosage forms. The dosage forms include tablets, capsules, troches, powders, solutions, suspensions, suppositories, or the like, preferably in unit dosage forms suitable for simple administration of precise dosages.

In the context of cancer, the terms “treat”, “treatment”, and “treating” can more specifically refer to the reduction or inhibition of the progression and/or duration of cancer, reduction of risk of developing cancer, the reduction or amelioration of the severity of cancer, and/or the amelioration of one or more symptoms thereof resulting from the administration of one or more therapies. In a specific embodiment, a patient that is at a high risk for developing cancer is treated. In specific embodiments, such terms refer to one, two, or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life. In certain embodiments, such terms refer to a stabilization or reduction in the cancer stem cell population. In some embodiments, such terms refer to a stabilization or reduction in the growth of cancer cells. In some embodiments, such terms refer to a stabilization or reduction in the cancer stem cell population and a reduction in the cancer cell population. In some embodiments, such terms refer to a stabilization or reduction in the growth and/or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in survival rate of a patient population. In further embodiments, such terms refer to an increase in the response rate, the durability of response, or number of patients who respond or are in remission. In some embodiments, such terms refer to a decrease in hospitalization rate of a patient population and/or a decrease in hospitalization length for a patient population.

As used herein, the term “therapeutically effective amount” in the context of cancer refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. Typically, an effective amount is provided according to a regimen. In an embodiment, the amount of a therapy is effective to achieve one, two, three or more of the following results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life. The term prophylactically effective amount refers to an effective amount administered to a subject either at risk of having cancer or who has already been treated for cancer and is administered to reduce relapse.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

Composition embodiments comprising a protein-polymer conjugate (e.g. polymer-conjugated Gal3C[T243C]) are included in the disclosure herein, and can be in the form of a solid, liquid or gas (aerosol). Typical routes of administration may include, without limitation, oral, topical, parenteral, sublingual, rectal, vaginal, ocular, intradermal, intratumoral, intracerebral, intrathecal, and intranasal. Parenteral administration includes subcutaneous injections, intravenous, intramuscular, intraperitoneal, intrapleural, intrasternal injection, directly into the lumen of the bladder, directly into the tumor, or infusion techniques. In a specific embodiment, the compositions are administered parenterally. In a more specific embodiment, the compositions are administered intravenously. Pharmaceutical compositions of the disclosure can be formulated so as to allow an antibody of the disclosure to be bioavailable upon administration of the composition to a subject. Compositions can take the form of one or more dosage units, where, for example, a tablet can be a single dosage unit, and a container of an antibody of the disclosure in aerosol form can hold a plurality of dosage units.

### 2. Summary of Results

This study shows the preparation and characterization of a soluble protein-polymer conjugate. The protein is the carbohydrate recognition domain of human galectin-3 (Gal3C), a lectin protein required in cell adhesion processes. Gal3C has been reported to enhance the benefits of anti-cancer therapeutics in two animal cancer models, however, the protein appeared to suffer from degradation after administration, limiting its utility ref. This specification describes a PEGylated form of this protein that shows increased robustness and stability, and methods for producing such proteins. The methods and products described here can help overcome the limitations observed in earlier studies.

PEGylation of the Galectin-3 carbohydrate recognition domain (Gal3C) creates thermal unfolding intermediate which increases the thermal stability of the protein. This method can produce proteins suitable for therapeutics with high specificity, formulation and storage stability, and biocompatibility. Proteins can be conjugated with synthetic polymers (i.e. PEG) to tune proteins properties. In order to improve methods, it is highly desirable to obtain more information on how the polymer-protein interactions affect their properties at the molecular level. Human Gal3C was used here as a model to study these interactions, but the same methods can be used with any protein as useful to the skilled artisan. Gal3C can be conjugated with PEG or other synthetic polymers using [T243C] to produce a conjugate functional for ligand recognition. Variable temperature CD spectroscopy was used to investigate unfolding. PEG prolongs the protein unfolding process by leading to formation of a polymer-dependent unfolding intermediate.

PEGylation of Gal3C[T243C] preserves its global structure but induces local perturbations of NMR spectra. NMR was performed with 15-35 μM ¹⁵N¹H protein or conjugate, 800 MHz, cryoprobe. Assignment from Gal3C (BMRB 4909) and full length Gal3 (BMRB 19491). PEGylation of Gal3C[T243C] perturbs NMR signals of a patch of residues at the protein's surface. Mapping of the crystal structure of Gal3C (PDB ID 4R9C) was performed, as well as τ_{c} from global measurements of backbone amides ¹⁵N R₁ and R₂ (556 Hz CPMG). PEGylation of Gal3C[T243C] slows down its rotational diffusion: PEG and the protein interact and do not behave as independent domains.

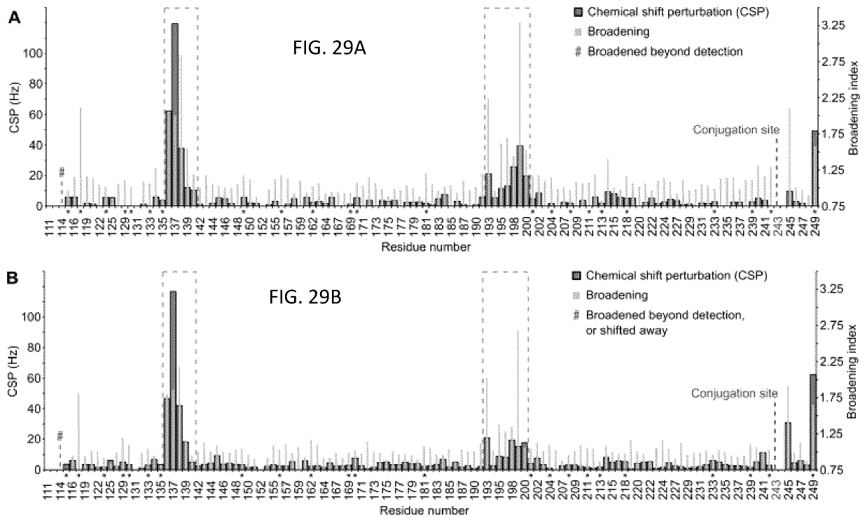

HDX and NOESY experiments were performed with Gal3C[T243C]-PEG, but PEG/Gal3C[T243C]interactions are relatively transient. PEG-induced perturbations are constant upon temperature increase. See FIG. 24.

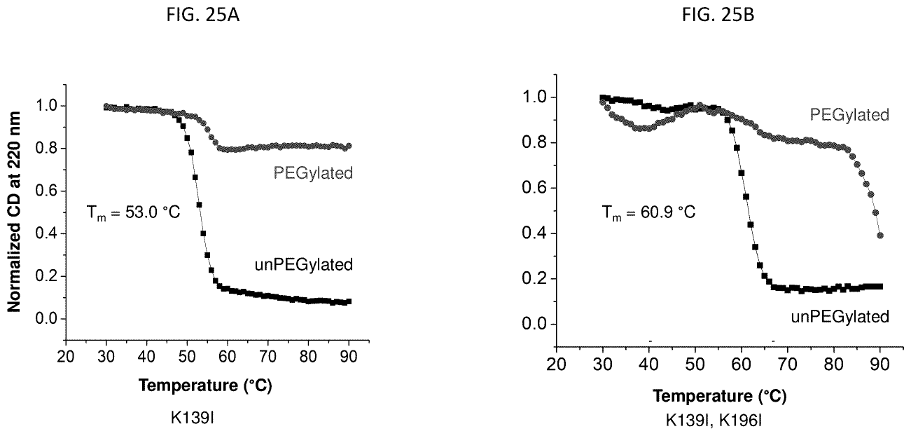

NMR of the freshly prepared conjugate was performed at 30° C. and after heating to 62° C. (3 min), then cooling down. Sample heating preserves the global fold but induces local perturbations, several of which overlap with NMR changes due to PEGylation. See FIG. 20.

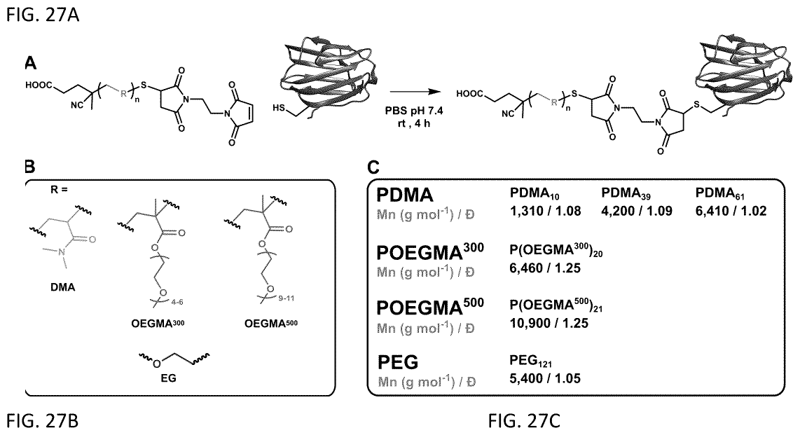

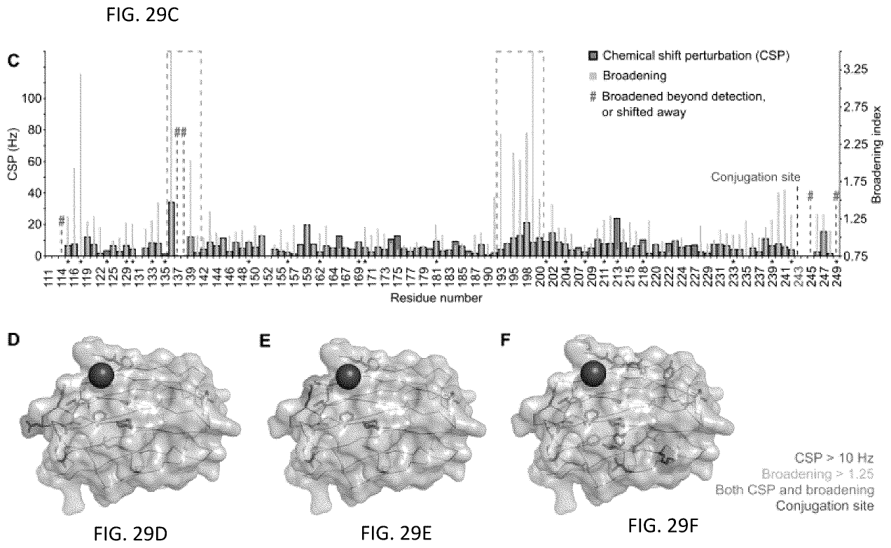

Lysines do not dominate PEG/Gal3C[T243C] interactions. Mutations in the region of PEG-induced perturbations affect the native protein stability, but not the observation of a thermal unfolding intermediate. Before lysine-to-isoleucine mutations, Gal3C[T243C]: Tₘ=58.0° C. Gal3C[T243C]-PEG: Tₘ₁=59.4° C., Tₘ₂=87.1° C. After an example lysine-to-isoleucine mutation, Gal3C[T243C,K139I]: Tₘ=52.7° C. Gal3C[T243C]-PEG: Tₘ₁=55.1° C., Tₘ₂=>90° C. PEGylation of Gal3C[T243C] or lysine-substituted variants does not alter its global structure and function, while it stabilizes it. PEG interacts loosely on the surface of Gal3C[T243C]. These interactions colocalize with changes that occur toward the unfolding intermediate. These interactions may stabilize regions of the protein that initiate the unfolding process. See also FIG. 22 and FIG. 25.

TABLE E1
Summary of Results.

| | Secondary | Gal3C[T243C] Relative to | Heated Relative to Freshly |
|---|---|---|---|
| Residue | Structure | Gal3C[T243C]-PEG | Prepared Gal3C[T243C]-PEG |
| L114 | N-terminus | Broadening beyond detection | — |
| I115* | N-terminus | — | Large signal perturbation |
| V116 | N-terminus | — | Shift and broadening |
| Y118* | β-strand | Broadening | — |
| N119 | β-strand | — | Large signal perturbation |
| G124* | Loop | — | Shift |
| R129* | Loop | — | Broadening |
| I134 | β-strand | Broadening | — |
| G136 | β-strand | Shift and broadening | Broadening |
| T167 | β-strand | Shift and broadening | Large signal perturbation |
| V138 | Loop | Shift and broadening | Large signal perturbation |
| K139 | Loop | Shift and broadening | — |
| N141 | Loop | Shift | — |
| R151 | Loop | — | Shift and broadening |
| F159 | β-strand | — | Shift |
| F163 | β-strand | — | Broadening |
| R169* | β-strand | — | Large signal perturbation |
| L177 | Loop | — | Large signal perturbation |
| V189 | Loop | — | Large signal perturbation |
| E193 | Loop | Shift, broadining, and possible splitting | Large signal perturbation |
| G195 | Loop | Shift and broadening | — |
| K196 | Loop | Shift and broadening | Shift |
| F198 | β-strand | Shift and broadening | Broadening |
| K199 | β-strand | Shift, broadining, and possible splitting | Shift |
| I200 | β-strand | Shift and broadening | — |
| V213 | Loop | — | Shift and broadening |
| N214 | Loop | Broadening | — |
| I240 | β-strand | Broadening | — |
| L242* | β-strand | Broadening | — |
| A245 | β-strand | Broadening | Large signal perturbation |
| M249* | C-terminus | Shift and broadening | — |

### 3. Embodiments of the Invention

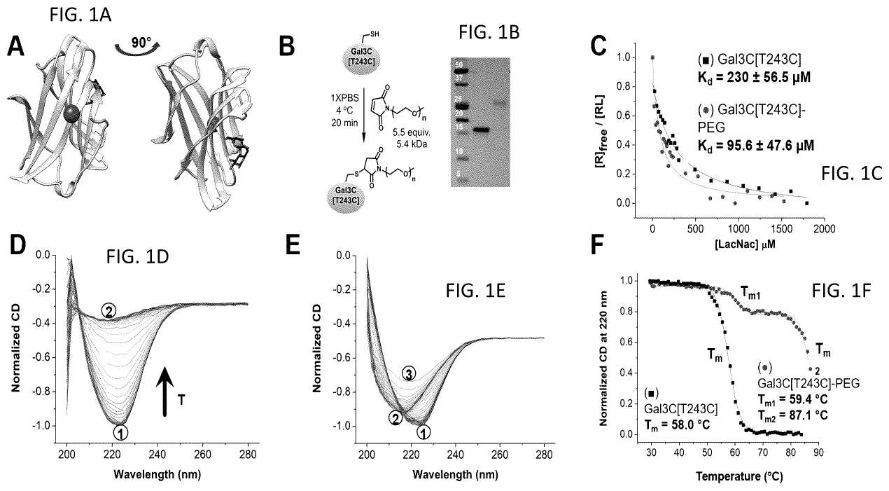

According to a certain embodiment, provided is a strategy to PEGylate the carbohydrate recognition domain of human galectin-3, Gal3C[T243C], via thiol-Michael addition of mPEG-maleimide (abbreviated herein as PEG) to an extrinsic cysteine resulted in highly pure and functional PEGylated protein. See FIG. 1. [As is noted elsewhere herein, other polymers can be used in place of PEG, and well as in conjunction with other variants of Gal3C.] Gal3C[T243C]-PEG retained the Gal3C[T243C] fold and showed similar to moderately higher affinities for two endogenous ligand. See FIG. 1C and Supplemental Table 1, below). This approach enabled scaled up production of PEGylated Gal3C[T243C] to generate sufficient quantities for structural and biophysical experiments and provides broad flexibility to further diversify the design of polymer-protein conjugates. Previous studies have generated a variety of conjugates by varying PEG molecular weight, positional isomer, PEG grafting density and attachment strategies (8-10,21,46-50). Combining the preparation strategy discussed here with NMR spectroscopic studies enables broad and systematic exploration of protein conjugate structure-function relationships.

Variable temperature CD spectroscopy showed that PEGylation of Gal3C[T243C] redirected the thermal unfolding pathway of the protein, resulting in two observed melting temperatures separated by a plateau representing an intermediate conformational state of the protein. See FIG. 1F. This intermediate state exhibited a slight shift in the 1 minimum of the normalized CD signal vs. wavelength spectra (FIG. 1E) but largely retained b-sheet secondary structure until irreversibly unfolding at a temperature significantly higher than the unfolding temperature of the unconjugated protein (FIG. 1E). Both the unconjugated and PEGylated Gal3C unfolded irreversibly. This suggests that PEGylation of Gal3C creates an unfolding intermediate that enables PEGylated Gal3C to remain soluble at elevated temperatures.

FIG. 1 shows the preparation of PEGylated Gal3C[T243C] and comparison of the function and temperature-dependent unfolding of Gal3C[T243C] and Gal3C[T243C]-PEG. In FIG. 1A, the crystal structure of Gal3C (PDB ID 4R9A) is shown in a ribbon representation in two orthogonal orientations. Position T243, which was replaced with a cysteine for chemical modification with PEG, is represented by a red sphere in both images. FIG. 1B shows the grafting-to conjugation scheme for PEGylation of Gal3C[T243C] using a thiol-Michael addition. FIG. 1C shows the equilibrium binding of LacNac to Gal3C[T243C] (black) and Gal3C[T243C]-PEG (red) measured by intrinsic tryptophan fluorescence. The plotted datasets are representative single experiments, and the reported K_{d} values are the average K_{d}±standard deviation for each protein determined from replicated experiments. FIG. 1D shows the superposition of normalized circular dichroism (CD) plots of Gal3C[T243C] measured from 30° C. (region ‘1’) to 90° C. (region ‘2’). The arrow and ‘T’ indicate an increase in temperature for individual experiments plotted in region ‘1’ to region ‘2’. FIG. 1E shows the superposition of normalized CD plots of Gal3C[T243C]-PEG measured from 30° C. to 90° C. As the temperature increased, three distinct regions were observed near 30° C. (region ‘1’), 70° C. (region ‘2’) and then 90° C. (region 3). In FIG. 1F, thermal unfolding of Gal3C[T243C] (black) and Gal3C[T243C]-PEG (red) was monitored by CD at 220 nm. For Gal3C[T243C], a single cooperative unfolding event was observed and for Gal3C[T243C]-PEG two separate unfolding events were observed, separated by a plateau spanning a temperature range from ˜70° C. to 80° C.

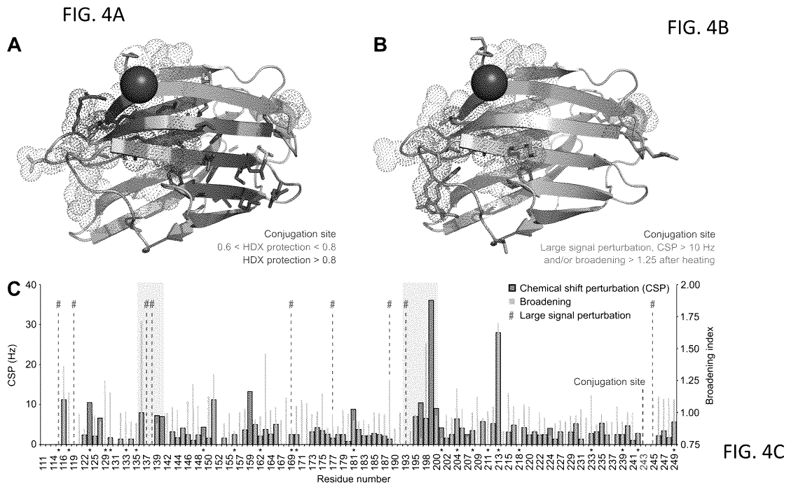

Variable temperature 2-dimensional [¹⁵N,¹H]-HSQC data show highly similar patterns of chemical shift changes and line broadening observed for PEGylated Gal3C[T243C] between 30° C. and 55° C. (see FIG. S13A and FIG S13B), the highest possible temperature that could be directly observed in these experiments. NMR experiments with Gal3C[T243C]-PEG heated to even higher temperatures and then showed more extensive chemical shift changes and line broadening, though predominantly in the same region (FIG. 4B, FIG. 4C, FIG. S13C). Overall, observations from NMR indicate that PEG remains relatively confined to a specific region on the surface of Gal3C[T254C]-PEG even as the temperature approached the first observed transition, suggesting that the localization of PEG is closely related to the thermal stability of the conjugated protein.

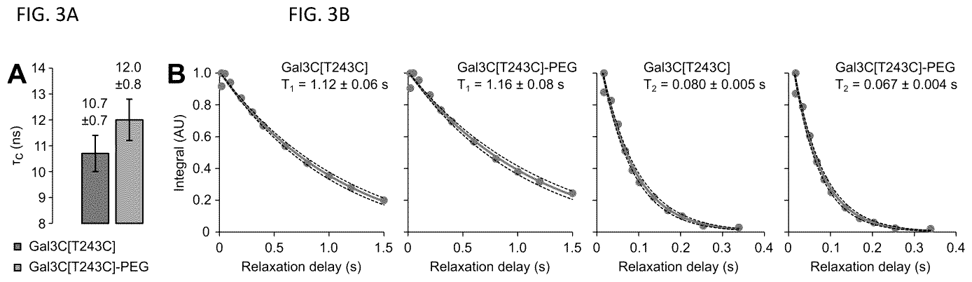

These data are of interest for refining models that describe the spatial organization of PEG with respect to the protein surface and the extent of non-covalent interactions between PEG and the protein, i.e. the ‘dumbbell’ and ‘shroud’ models. Localization of chemical shift perturbations and line broadening observed by NMR suggest that protein-PEG interactions occur in a specific region of the protein surface. An analogous observation was made in an earlier NMR and x-ray diffraction study with PEGylated plastocyanin (51). The NMR-observed perturbations presented here imply that PEG does not exhibit a random coil structure, but rather has collapsed upon the protein surface in a defined region. The slower rate of rotational diffusion (t_{c}) for Gal3C[T243C]-PEG also supports that PEG does not behave as an independent domain (FIG. 3A). This observation is somewhat in contrast with the same earlier NMR and x-ray diffraction study of PEGylated plastocyanin, which concluded there were minimal to no interactions between the protein and conjugated PEG. This conclusion was based on comparison of NMR linewidths between the PEGylated and unconjugated proteins and also from the observation that PEG was not observed in crystals of the protein, but it lacked an explicit comparison of diffusion properties of the two molecules. Altogether, these results are more in line with literature data of PEGylated human Interferon-at a , which reported that PEG formed a “shield” for the protein but did not permanently cover the protein surface.

FIG. 3 presents a comparison of the rotational diffusion rates of Gal3C[T243C] and Gal3C[T243C]-PEG. FIG. 3A shows rotational correlation times (τ_{c}) of Gal3C[T243C] (blue) and Gal3C[T243C]-PEG (orange) at 30° C. determined from ¹⁵N relaxation data shown in (B). Error bars indicate the 95% confidence intervals. FIG. 3B shows global ¹⁵N longitudinal and transverse relaxation times (T₁ and T₂, respectively) of amide groups in the structured core of Gal3C[T243C] and Gal3C[T243C]-PEG. Blue points represent normalized integral values obtained for each relaxation delay, and the solid blue line is the non-linear regression used to quantify T₁ and T₂. The dotted lines indicate the 95% confidence interval from the regression procedure.

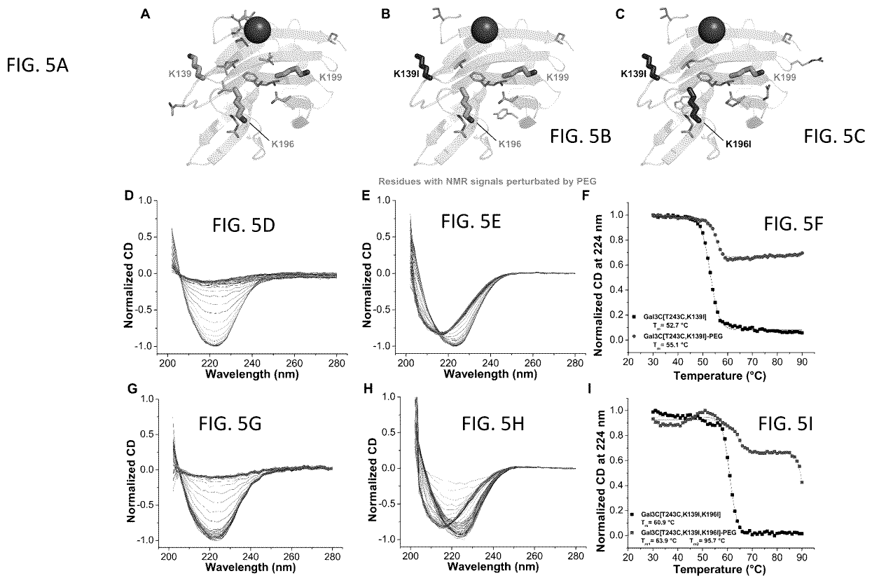

An important consideration in designing de novo protein-polymer conjugates is whether specific amino acid types drive the spatial organization of PEG on the protein surface. Earlier computational studies of PEGylated proteins proposed that lysines could play an important role in orienting PEG on a protein through hydrogen bonding (24,44,45). In the present study, two lysines that bordered a region of the protein surface affected by PEGylation were replaced. Replacing these lysines with isoleucines resulted in partially altering the region of PEG-Gal3C interactions (FIG. 5). Thermal unfolding data with the corresponding variant proteins appeared distinct from Gal3C[T243C]-PEG, though benefits of PEGylation (i.e. increased thermal stability) and the formation of unfolding intermediates were still observed (FIG. 5). These data suggest that protein-PEG interactions are likely influenced by multiple amino acid types and that even subtle reorientation of PEG on the protein surface can impact protein thermal unfolding properties.

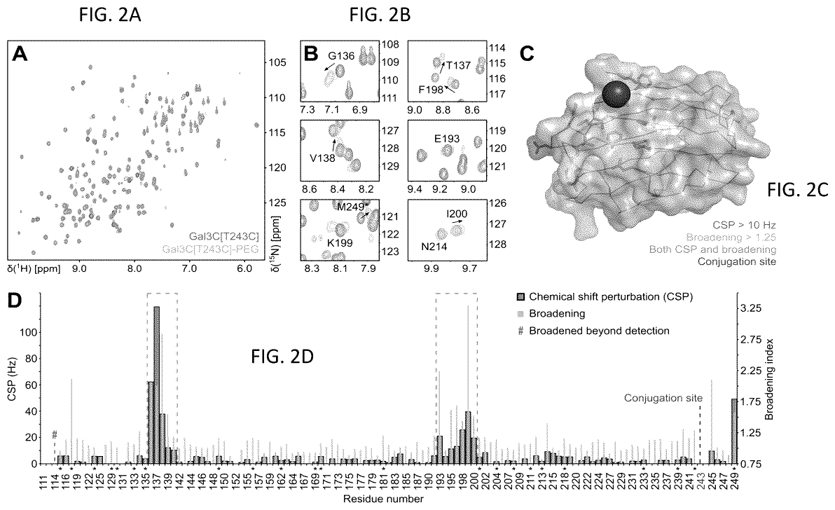

In the present study, integrating quantitative NMR data with correlative thermal melting and functional assays enabled us to develop an atomic view of the nature of interactions between the carbohydrate recognition domain of human galectin-3 and covalently attached polyethylene glycol. Mono-PEGylating human Gal3C[T243C] at position 243 was observed to lead to increased thermal stability mediated through formation of a thermal unfolding intermediate with similar, but distinct, secondary structure. (FIG. 1D-F) Two-dimensional NMR correlation spectra indicated PEG-dependent perturbations are local to the site of PEGylation. (FIG. 2) In parallel, NMR hydrogen-to-deuterium exchange data and diffusion measurements support a “shroud-like” solution conformation of the PEG with respect to Gal3C. (FIG. 4A and FIG. S12)

While protein-PEG interactions are likely complex and involve multiple amino acid types, the presence of positively charged amino acids, particularly lysines, near the border of the PEG-Gal3C interface suggested they played a special role in determining the localization of PEG on the Gal3C surface. Interestingly, for lysines identified by highly perturbed chemical shifts in the NMR data of Gal3C-PEG, substitution with isoleucines led to observable changes in PEG-dependent perturbations on the protein surface and subsequent changes to the thermal unfolding profiles. For these variants, PEG-dependent chemical shift perturbations were still observed, suggesting that PEG still interacted with the surface of Gal3C variants, though at potentially different positions than Gal3C[T243C]. Also, for these lysine variants, the formation of the unfolding intermediate and higher melting temperatures with the protein-PEG conjugates were observed. Replacement of just one lysine, K139, led to an even higher measured transition and melting temperature that was beyond the upper temperature limit of our instrumentation. These observations suggest that protein-PEG interactions are likely facilitated by multiple amino acid types, supporting the robust formation of conjugates with altered chemical properties. At the same time, seemingly subtle alterations of protein-PEG interactions appeared to significantly alter the properties of the conjugate. (FIG. 5 and FIG. 25)

In this work, NMR spectroscopy played a particular important role in revealing both local, atomic-resolution information on protein-polymer conjugates and measurements of the overall diffusion properties of conjugated proteins.

Therefore, this specification describes (see Examples), with experimental detail a structural model of a protein-polymer bioconjugate. This model is used both to understand how PEGylation alters the thermal stability of the protein and to rationally select mutations that allow control over PEG-protein interactions. Biologics have profoundly impacted numerous medical fields but their development is limited by the need for proteins to survive the challenging environment inside the human body. Polymer-based protein engineering has emerged as one of the most promising approaches to overcome this problem. Protein PEGylation, in particular, has been successfully used to produce over 30 FDA-approved protein-polymer drugs, many of which are successful anti-cancer therapeutics. However, wider application of protein PEGylation is hindered by the lack of a precise structural view of protein-polymer interactions that give rise to their beneficial properties, as demonstrated in recent studies with failed enzyme therapeutics.

This study provides an atomic level view into the structure of PEGylated human galectin-3, a galactoside binding protein critical to cellular adhesion and angiogenesis, which is both a target for small molecule drugs and a biologic of interest for cancer treatment. These techniques can be used with and for other proteins as well. The data provide new insights into the mechanism by which the covalently attached polymer alters the structure, dynamics, and thermal unfolding behavior of the conjugated protein. Importantly, PEGylation results in the generation of a key intermediate conformational state critical for improving the thermal stability of the PEGylated protein, which is presented for the first time here. Using NMR spectroscopy, one can relate the formation of this state to the spatial location of PEG near the surface of galectin-3, then use the molecular model to introduce judiciously placed mutations, i.e., rationally-selected residues, that when conjugated to a polymer such as PEG, disrupt the PEG-protein interaction interface and alter the thermal unfolding properties of the conjugated mutants.

In summary, this work provides a substantial revision to the current understanding of protein-polymer interactions than those put forward through low-resolution experimental techniques. Moreover, the results provide a critical experimental foundation for the development of improved theories for engineering protein-polymer conjugates with novel properties. The research strategy used here establishes a biophysical toolbox that can be generally applied to characterize protein-polymer interactions with atomic level precision in a comprehensive manner.

### 4. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention. For additional information, see FIG. P1 and FIG. P2 in Appendix A and Appendix B.

### Example 1: General Methods For Examples 2-7

### A. Molecular Biology Reagents

All primers were ordered from Integrated DNA Technologies™ (IDT). Phusion Hot Start II HF Polymerase and HF Reaction Buffer were used for PCR mutagenesis (Thermo Fisher™). NcoI, DpnI, T4 DNA Ligase and CutSmart Buffer (New England Biolabs™) were used in the preparation of all Gal3C T243C variant plasmids. A Wizard SV™ Gel and PCR Clean-Up System and Wizard Plus SV™ Minipreps DNA Purification System (Promega™) were used for agarose-gel DNA extraction and plasmid isolation. Origami B(DE3) competent E. coli cells (Novagen™) were used for all protein expression. HisPur™ Cobalt Resin was used for IMAC affinity purification (Thermo Fisher™).

### B. Solvents and Reagents

Dichloromethane (DCM, 99.6%), diethyl ether (ACS grade), 29% NH₄OH, triethyl amine (TEA, 99%), and chloroform (CHCl₃, ACS grade) were purchased from Fisher Chemical™. Maleic anhydride (99%) and acetic anhydride (Ac₂O, 99%) was obtained from Oakwood Chemical. Poly(ethylene glycol) mono methyl ether (mPEG, Mₙ=5k g/mol) was purchased from Sigma Aldrich™. Methanesulfonyl chloride (MsCl, >99%) was from TCI America. ¹⁵NH₄Cl was purchased from anhydrous (NaAc), dextrose (D-glucose) anhydrous, and [alpha]-lactose monohydrate were obtained from Fisher Chemical™. N-acetyllactoamine was purchased from Biosynth Carbosynth.

### C. Construct Design

### 1. Preparation of the Gal3C Plasmid:

The galectin-3 gene is contained in an open reading frame of a pET-21d(+) vector between the XhoI and NcoI restriction Cambridge Isotope Laboratories™. Sodium acetate sites was gifted from the Hudalla Lab of University of Florida Department of Biomedical Engineering. A variant of galectin-3 containing residues A111 to E252 of the carbohydrate recognition domain (Gal3C) was created by restriction digest using the Ncol sites flanking the N-terminal domain sequence. A second NcoI restriction site was inserted into the gene after proline 105 using the overlapping NcoI-FWD and NcoI-REV primers in Table S4.

The PCR product with the brightest band was isolated from a 1% agarose gel and subjected to DpnI digestion before digesting with NcoI to remove the N-terminal sequence. The NcoI digest products were separated on a 1% agarose gel and the larger product was isolated and ligated. The final product was transformed into One Shot TOP10 chemically competent E. coli cells from Invitrogen™ and the sequence was confirmed by Genewiz™.

### 2. Preparation of the Gal3C[T243C] Plasmid:

Primers T243C-FWD and T243C-REV (see Table S4, below) were used for site-directed mutagenesis with the Gal3C plasmid as the template DNA and Phusion Hot Start II HF DNA Polymerase. The PCR product was separated on a 1% agarose gel, and the brightest band isolated and subjected to DpnI digestion before transforming into One Shot TOP10 chemically competent E. coli cells and then sequenced with Genewiz™.

TABLE S4
Oligonucleotide primers used for the generation of Gal3C[T243C] Variants

| | | | SEQ ID |
|---|---|---|---|
| Name | Description | Sequence 5″ - 3″ | NO |
| NcoI-FWD | NcoI insertion overlapping | CCCCTCCATGGCTGGGCCACT | 1 |
| | forward primer | GATT | |
| NcoI-REV | NcoI insertion overlapping | CCAGCCATGGAGGGGCGCCA | 2 |
| | reverse primer | TAGGG | |
| T243C-FWD | T243C forward mutation | AGACCTCTGCAGTGCTTCATA | 3 |
| | | TACC | |
| T243C-REV | T243C reverse mutation | AGCACTGCAGAGGTCTATGT | 4 |
| | | CAC | |
| K139I-FWD | K139I forward mutation | CTGATAACAATTCTGGGCAC | 5 |
| | | GGTGATTCCCAATGCAAACA | |
| | | GAATTGCTTTA | |
| K139I-REV | K139I reverse mutation | TAAAGCAATTCTGTTTGCATT | 6 |
| | | GGGAATCACCGTGCCCAGAA | |
| | | TTGTTATCAG | |
| K196I-FWD | K196I forward mutation | TCGGTTTTCCCATTTGAAAGT | 7 |
| | | GGGATTCCATTCAAAATACA | |
| | | AGTACTGGTT | |
| K196I-REV | K196I reverse mutation | AACCAGTACTTGTATTTTGAA | 8 |
| | | TGGAATCCCACTTTCAAATG | |
| | | GGAAAACCGA | |
| K199I-FWD | K199I forward mutation | CCATTTGAAAGTGGGATTCC | 9 |
| | | ATTCATTATACAAGTACTGGT | |
| | | TGAACCTGAC | |
| K199I-REV | K199I reverse mutation | GTCAGGTTCAACCAGTACTT | 10 |
| | | GTATAATGAATGGAATCCCA | |
| | | CTTTCAAATGG | |

Provided below is SEQ ID NO: 11. This is the Gal3C[T243C] sequence was used for the chemical conjugation with PEG and other polymers as described herein (e.g. see Examples).

M A G P L I V P Y N L P L P G G V V P R M L I T I
L G T V K P N A N R I A L D F Q R G N D V A F H F
N P R F N E N N R R V I V C N T K L D N N W G R E
E R Q S V F P F E S G K P F K I Q V L V E P D H F
K V A V N D A H L L Q Y N H R V K K L N E I S K L
G I S G D I D L T S A S Y C M I L E H H H H H H

Provided below is

SEQ ID NO: 12 (amino acid sequence of homo sapiens
Gal3C, T243 bolded):
MADNFSLHDALSGSGNPNPQGWPGAWGNQPAGAGGYPGASYPGAYPGQAP
PGAYPGQAPPGAYPGAPGAYPGAPAPGVYPGPPSGPGAYPSSGQPSATGA
YPATGPYGAPAGPLIVPYNLPLPGGVVPRMLITILGTVKPNANRIALDFQ
RGNDVAFHFNPRFNENNRRVIVCNTKLDNNWGREERQSVFPFESGKPFKI
QVLVEPDHFKVAVNDAHLLQYNHRVKKLNEISKLGISGDIDLTSASYTMI
SEQ ID NO: 13 (Threonine relating to T243 bolded)
M A G P L I V P Y N L P L P G G V V P R M L I T I
L G T V K P N A N R I A L D F Q R G N D V A F H F
N P R F N E N N R R V I V C N T K L D N N W G R E
E R Q S V F P F E S G K P F K I Q V L V E P D H F
K V A V N D A H L L Q Y N H R V K K L N E I S K L
G I S GD I D L T S A S Y T M I L E H H H H H H

### 3. Preparation of the Gal3C[T243C,K139I], Gal3C[T243C,K139I,K196I] and Gal3C[T243C,K139I,K196I,K199I] Plasmids

For generating the Gal3C[T243C] variants with lysine replacements, PCR-based site-directed mutagenesis was used (AccuPrime™ Pfx Supermix, Invitrogen™). Primers for these series of mutagenesis are listed in Table S4, and the resulting plasmid sequences were confirmed with Genewiz™.

D. Synthesis of monomethoxy-PEG5.4k-maleimide (PEG)

Each polymer synthesis step was followed by ¹H NMR for samples measured in CDCl₃. Spectra were recorded on a Bruker TM Ascend 400 MHz spectrometer equipped with TopSpin™ v3.5.7 software. NMR data were processed using MestReNova™ x64 v4.0 software.

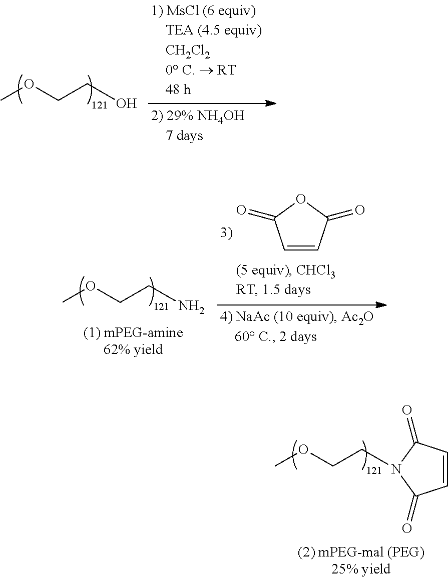

Step 1: Production of the mPEG-Ms Intermediate

Bulk poly(ethylene glycol) monomethyl ether, Mₙ=5K (mPEG) (1 equiv., 31.85 g) was placed in a round bottom flask and dried under high-vacuum while stirring at 60° C. for 1.5 hours. The dried mPEG was dissolved in 75 mL DCM and triethylamine (TEA) (4.51 equiv., 4 mL) was added to the solution. The mixture was placed in an ice bath and purged with argon for 30 minutes while stirring. Methanesulfonyl chloride (MsCl) (6.09 equiv., 3 mL) was dissolved in 25 mL of DCM and dripped into the mPEG solution over the course of 20 minutes using an addition funnel. White precipitate was immediately observed upon the addition of the MsCl. After two hours, about 75 mL of DCM was added to the reaction to dilute it. After 48 hours, the reaction was concentrated down to approximately 100 mL with rotary evaporation and appeared yellow in color. The solution was washed 2× with 5% HCl (150 mL each wash), then once with brine, in a separatory funnel. The organic layer was dried over Na₂SO₄ and gravity filtered to remove the salt. Cold ether (900 mL) was stirred in an Erlenmeyer flask and the organic layer was slowly added to precipitate the mPEG-Ms product as a fluffy white powder. mPEG-Ms was collected by vacuum filtering the solid from the ether slurry.

Step 2: Production of (1) mPEG-amine

For this step we adapted a previously published protocol from Barnes, et al. The crude mPEG-Ms product was immediately dissolved into 700 mL of 29% NH₄OH in a 1 L round bottom flask, capped with a needle-pierced septum, and stirred for four days at room temperature. The flask was then opened to the atmosphere and stirred at room temperature for an additional 3 days. The reaction was split into halves and each half was extracted with DCM twice in a separatory funnel. The DCM layer was then evaporated and the PEG-amine product dried under high vacuum with 19.82 g recovered (62% yield).

Step 3: Production of mPEG-Acid Intermediate

PEG-amine (1 equiv., 2.45 g) was dissolved in 20 mL of CHCl₃ in a sealed addition funnel and purged with argon. Maleic anhydride (5 equiv., 0.196 g) was dissolved in 5 mL of CHCl₃ in a round bottom flask and purged with argon. The polymer solution was dripped into the maleic anhydride over the course of 30 minutes. After 1.5 days (35 hours), the PEG-acid intermediate was precipitated into 125 mL cold ether and left at −20° C. overnight. The desired product was isolated with Buchner funnel filtration and dried on high vacuum to yield 2.37 g of material (2.37 g, 97% yield).

Step 4: Production of PEG-mal (PEG).

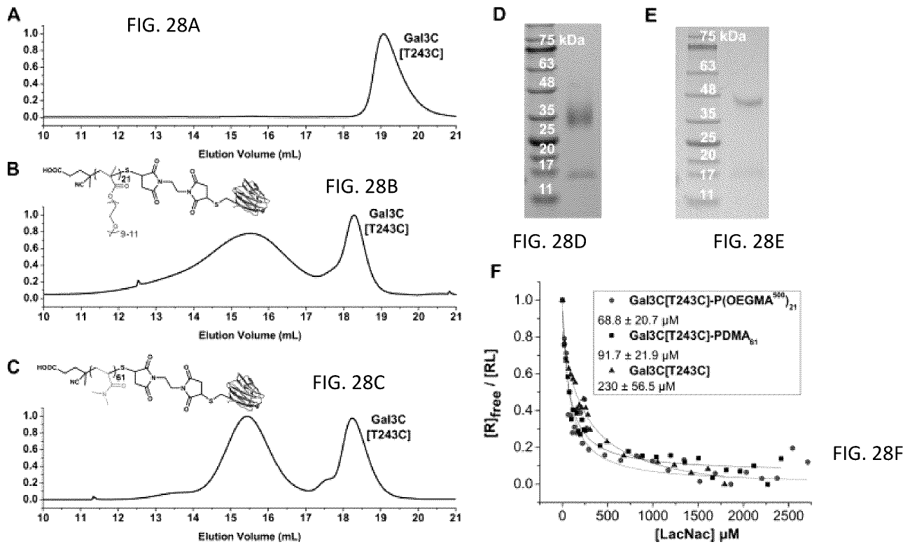

Immediately following drying, the product from step 3 (1 equiv., 2.22 g) was loaded into a round bottom flask and dissolved in 27 mL of neat acetic anhydride. Sodium acetate (10.4 equiv., 0.380 g) was added to the flask and did not fully dissolve. The mixture was then purged with argon and sealed. The reaction was stirred at 60° C. for two days. The crude reaction was precipitated into ether to recover the PEG-mal product. The precipitated PEG-mal was dialyzed against 4 L of water for one week (1 kDa MWCO membrane) to remove remaining acetic anhydride. After lyophilizing, the product (1.233 g) was recovered. Based on ¹H NMR spectra in CDCl₃ (FIG. 23), 47% of the recovered material was the PEG-mal product, corresponding to a final yield of 25%. FIG. 23 presents a ¹H NMR spectrum of mPEG-maleimide (PEG) in CDCl₃ measured at room temperature and 400 MHz 1 H precession frequency. Relative signal integrals and assignments are shown.

### Alternative Polymer Conjugation

In other embodiments, polymers are functionalized on the end-group as follows:

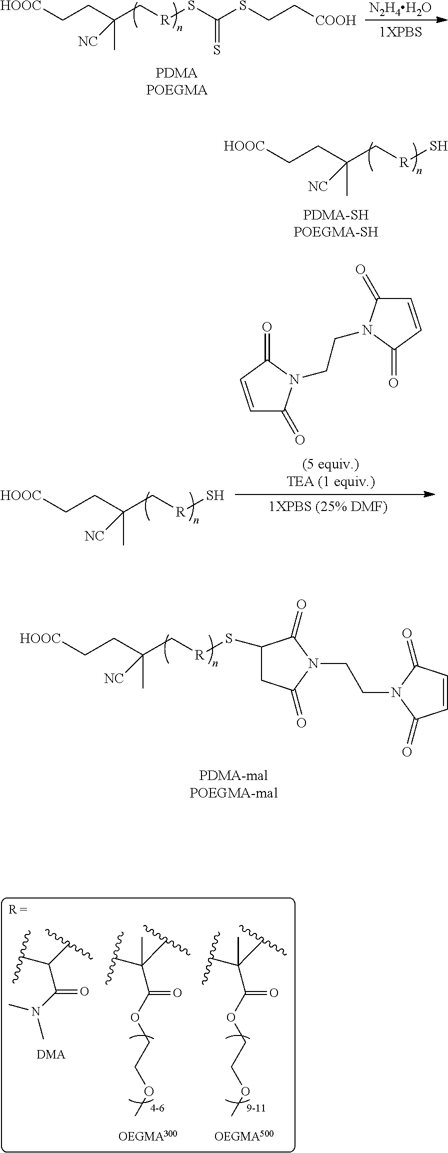

The above functionalized PDMA-mal and POEGMA-mal polymers can alternatively be used in place of the PEG-mal for conjugation of Gal3C according to the techniques described herein.

### E. Protein Production

### 1. Expression of Gal3C and Gal3C[T243C] and Gal3C[T243C] Variants Containing Lysine Replacements.

Plasmids containing Gal3C, Gal3C[T243C], Gal3C[T243C,K139I], Gal3C[T243C,K139I,K196I] and Gal3C[T243C,K139I,K196I,K199I] were transformed into Origami B(DE3) competent cells for expression and transferred to LB agarose plates containing 100 μg/mL kanamycin and ampicillin. Glycerol stocks created from selected colonies were used for all subsequent protein production. Large scale protein production of unlabeled Gal3C[T243C] variants proceeded as follows. First, 5 mL of LB media containing 100 μg/mL kanamycin and ampicillin was inoculated with a stab of the glycerol stock and grown overnight at 37° C. and 250 rpm. Each 0.5 L culture of 2×YT media was inoculated from one 5 mL overnight culture in a 2.8 L flask. Cultures were grown at 37° C. until reaching an OD₆₀₀ of 0.7-0.9. Expression was then induced with 0.5 mM IPTG and the temperature lowered to 18° C. for a total expression time of 18 hours.

2. Expression of [u-¹⁵N]-Gal3C[T243C], [u-¹⁵N]-Gal3C[T243C,K139I] and [u-¹⁵N]-Gal3C[T243C,K139I,K196I]:

Expression conditions for the stable-isotope labeled protein were similar to the unlabeled expression of Gal3C[T243C] variants with the following changes. For each 0.5 L cell culture, a 75 mL starter culture was prepared in LB media in a baffled 125 mL flask from glycerol stocks. After 12 hours, the starter cultures were centrifuged at 5,000 rpm for 20 minutes, the media was decanted and the cell pellet was resuspended in M9 minimal media containing 4 g L⁻¹ glucose and 1 g L^{-1 15}NH₄Cl, FeCl₃ (0.05 mM, prepared in 100 mM citric acid), thiamine hydrochloride (0.02%) and standard M9 minimal media salts. Total expression time was 19.5 hours. Expression temperature and IPTG concentration was the same for unlabeled Gal3C[T243C].

### F. Protein Purification

The thawed cell pellet from 3 L of culture was resuspended in approximately 70 mL of cold 1×PBS, 10 mM DTT, and 2× Protease Inhibitor, and lysed with three passages through a cell disruptor (Pressure Biosciences™) at 25 kpsi. The lysate was clarified by centrifuging at 30,000×g for 1 hour. Then the lysate supernatant was filtered with 0.45 μM GF+PES syringe filters and mixed with cobalt IMAC resin at the ratio of 0.5 mL per 2 L of original culture volume in conical tubes. The filtered lysate and resin were rotated at 4° C. for 20-30 minutes before loading onto an empty 25 mL gravity column. The resin was then washed with 1×PBS (50 mL), 1×PBS+500 mM NaCl (50 mL), and 1×PBS+5 mM imidazole (30 mL). The protein was eluted with 5 mL of 1×PBS+75 mM imidazole after first allowing the buffer to enter the resin bed and incubated without stirring or rotating the column for 15 minutes. 0.5 mL fractions were collected until the protein concentration in eluted fractions dropped below 20 μM. Typical yields for Gal3C[T243C] expressed in minimal media were 2.1 mg purified Gal3C[T243C] per liter of culture.

### G. Preparation of PEGylated Gal3C[T243C], Gal3C[T243C,K139I], and Gal3C[T243C,K139I,K196I]

PEG_{5.4k}-maleimide (11 equiv., 5.5 equiv. of functionalized PEG) was dissolved in 0.3-0.5 mL of PBS buffer and loaded into a 100 mL round bottom flask with a stir bar. Gal3C[T243C] was buffer exchanged into PBS immediately before conjugation using an AKTA FPLC system equipped with a 5 mL HiTrap Sephadex™ G-25 Desalting column (Cytiva™) Then 1 equivalent of Gal3C[T243C] or Gal3C[T243C] variant was loaded into a 125 mL addition funnel and dripped into the polymer solution over the course of 20 minutes at 4° C. while stiffing gently. The reaction was allowed to proceed for 20 minutes and then was quenched with 10 mM DTT.

To remove excess PEG from the Gal3C[T243C] PEGylation reaction, the crude products were loaded onto 0.5-1.0 mL of lactose-agarose affinity resin at a ratio of at least 10 mg of protein: 1 mL of resin and added into Eppendorf™ tubes. Tubes containing the resin and crude reaction were rotated at 4° C. for 2 hours to maximize binding and protein recovery. The resin and reaction mixture were loaded into an empty 15 mL gravity column. The flow-through was collected and passed through the column twice to improve the final yield. The column was washed with 50 mL (50-100×CV) of cold PBS to remove excess PEG-mal. 1 column volume of elution buffer containing cold PBS and 30 mM lactose was added to the resin and one to two 0.5 mL fractions were collected. Then an additional 3.0 mL elution buffer was added to the resin and incubated with the resin bed for 15 minutes before six elution fractions of 0.5 mL each were collected. Fractions containing the highest amounts of protein were pooled and concentrated to ⅓ of the total volume using a speedvac turned on for 2-minute intervals with a heated sample chamber to prevent freezing.

Because the eluate from the lactose-affinity purification step contained both Gal3C[T243C] and Gal3C[T243C]-PEG, size exclusion chromatography (SEC) was used to isolate the Gal3C[T243C]-PEG product. SEC was performed with an AKTA Go system (Cytiva™) and a Superdex™ 200 Increase 10/300 GL column (Cytiva™) equilibrated with buffer containing either 1×PBS for ligand binding and CD experiments or a 20 mM sodium phosphate buffer with 30 mM NaCl, pH 6.90 for NMR experiments. Baseline separation of Gal3C[T243C]-PEG was achieved and fractions were pooled for subsequent experiments. The same PEGylation and purification steps were followed for each of the Gal3C lysine variants.

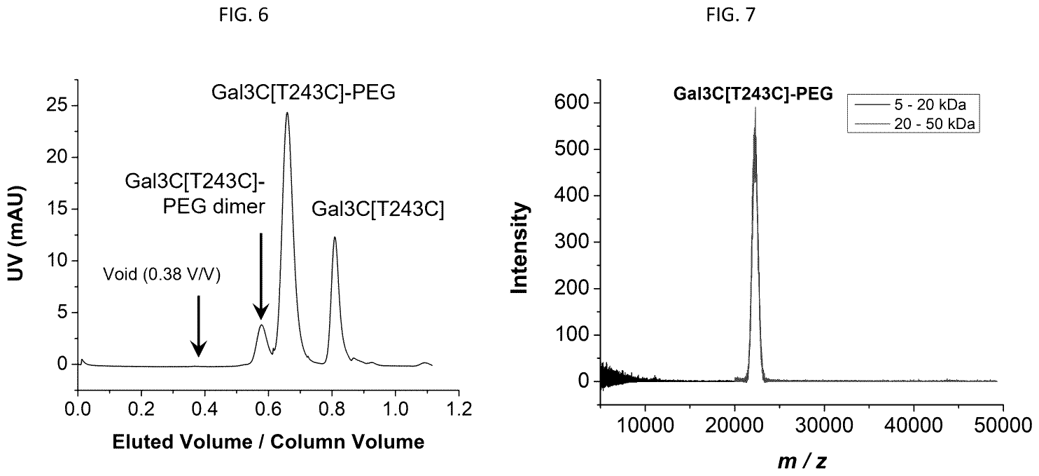

FIG. 6 presents a size exclusion chromatogram from a representative sample preparation of Gal3C[T243C]-PEG. The sample was applied after removal of excess maleimide-PEG. Analytical separation of Gal3C[T243C]-PEG and Gal3C[T243C] was achieved, permitting isolation of the conjugated protein. The separated peaks for Gal3C[T243C]-PEG and Gal3C[T243C] are annotated. See additional details for the method herein.

### H. Mass Spectrometry

Matrix assisted laser desorption/ionization time-of-flight (MALDI-TOF) mass spectrometry was performed on a Bruker Daltonics™ Microflex LRF mass spectrometer (Bruker Daltonics™, Breman, Germany) operated in linear, positive ion mode with an N2 laser. The laser power was set to 50% or greater to reach a threshold level required to generate signal. Signals were summed to sufficient intensity. Instrumental calibration was performed for 5-20 and 20-50 kDa linear ranges via methods specified by the manufacturer. Protein Calibration Standards I and II purchased from Bruker Daltonics™, which bracketed the range of sample molecular weights. Signals in the range of 4-66 kDa were assigned for calibration. Solutions containing either unconjugated or conjugated protein were prepared at 15-60 μM in 1×PBS or 20 mM sodium phosphate buffer with 30 mM NaCl for samples spotted on the target. The matrix was 10 mg/mL (saturated) sinapinic acid in 1:1 ACN/0.1% TFA in water. Samples for spotting on a polished MALDI target plate were mixed together at a ratio of 1:1 protein:matrix. At least 3×1 μL of the mixture was spotted on the target plate and allowed to dry before analysis.

### I. Ligand Binding Assay

Equilibrium binding data were recorded using a protocol adapted from a previous procedure from Restuccia, et al (38). Intrinsic tryptophan fluorescence was monitored using a Cary Eclipse™ Fluorescence Spectrophotometer (Agilent) operating with Cary Eclipse™ WinFLR software version 1.2 in ‘Scan Mode’. The instrument method parameters were as follows: excitation wavelength was 280 nm, the acquisition range was from 290 nm to 440 nm and the photomultiplier tube sensitivity was set to ‘medium’. Samples were prepared at final concentrations of 1 μM for Gal3C[T243C] or 5 μM Gal3C[T243C]-PEG in 800 ∥L, in PBS for each titration. Samples were pipetted into 0.7×0.7 cm quartz cuvettes with a micro stir bar. Each new experiment was first zeroed against PBS to remove the small background signal from the buffer. Stock solutions of ligands in PBS (66 mg⁻¹ mL⁻¹ lactose or 34 mg⁻¹ mL⁻¹ LacNac) were titrated into the solution such that the first addition of ligand was at least 10× the concentration of the protein. Increasing concentration of ligands were titrated until reaching a concentration of 10 times the theoretical K_{d}, recording at least 20 data points per titration.

### J. Functional Assay Data Fitting

The general binding equation describing reversible binding of Gal3C (R) to the ligand (L) is described:

R+L [symbol] RL

Where the equilibrium binding constant (K_{d}) is defined in the following relation:

$${K}_{d} = \frac{[ {R}_{eq} ] [ {L}_{eq} ]}{[ {RL}_{eq} ]}$$

A low and fixed [R] and a wide range of [L] surpassing the theoretical K_{d} by 10× was chosen for the functional assay. A rearrangement of this equation, considering fractional occupancy of the receptor, can be written in terms of [L] and normalized by the y value at the data start and end points as listed immediately above.

$$f ( L ) = \frac{{[ R ]}_{free}}{{[ R ]}_{total}} = \frac{{[ R ]}_{free}}{( {[ R ]}_{free} + [ RL ] )} = Start + ( End - Start ) \frac{[ L ]}{{K}_{d} + [ L ]}$$

The above equation, labeled as the Hill 1 equation in Origin v8.5 software, was used to fit normalized equilibrium binding data. For the Gal3C[T243C]-PEG binding data, fixed parameters included the Start and End values as 1 and 0, respectively. Replicated experiments with a fit error of ≤30% were used to the calculate the average K_{d}±standard deviation with a total of 3 independent replicated experiments.

### K. Circular Dichroism (CD) Spectroscopy Experiments and Data Analysis

CD spectroscopic data were recorded with an Applied Photophysics™ Chirascan spectrophotometer equipped with a Quantum Northwestern Peltier temperature control device and operating with Chirascan v4.7.0 and Pro-Data™ Viewer software. Samples were prepared in PBS at final concentrations of 10 μM. All samples were contained in 2 mm tapered quartz cuvettes, and a temperature probe was inserted through the cuvette cap to monitor the sample temperature in real time. The following parameters were used for constant temperature experiments: CD signal measured in millidegrees, bandwidth of 1.0 nm, wavelength scan from 200-280 nm, step size of 2.0 nm. Spectra were background subtracted against the sample buffer. For thermal unfolding experiments, a linear thermal ramp was applied from 30° C. to 90° C. at 1° C. per minute. All spectra were measured in triplicate, and data were normalized to account for slight differences in protein concentration.

To calculate melting temperatures of unconjugated Gal3C, Gal3C[T243C], and Gal3C[T243C] variants containing lysine replacements, the normalized data were fit to a Boltzmann sigmoidal function in Origin v8.5. For the data fitting, no parameters were fixed, and the ‘x’ parameter defined as the center of the data was interpreted as the melting temperature (Tₘ). Variable temperature CD data of PEGylated Gal3C[T243C] and Gal3C[T243C] variants were fit with a sum of two Boltzmann sigmoidal functions, as shown below:

$$f ( y ) = A 3 + \frac{A 1 - A 2}{1 + {e}^{\frac{( x - x 1 )}{dx 1}}} + \frac{A 2 - A 3}{1 + {e}^{\frac{( x - x 2 )}{dx 2}}}$$

where ‘A1’, ‘A2’, and ‘A3’ represent three plateaus of the CD signal, ‘dx1’ and ‘dx2’ are the change in x between the plateaus for each melting transition, and ‘x1’ and ‘x2’ are the two Tₘ values extracted by the fit for the first and second unfolding transitions, respectively. Because the third plateau, A3, occurred at temperatures higher than the experimentally accessible range, this value was estimated. A single wavelength for plotting on the y-axis was selected by calculating the wavelength of greatest CD signal change for each PEGylated construct.

To record a CD spectrum of Gal3C[T243C] with free PEG, a stock solution of methoxy-PEG (mPEG) in 1×PBS was added immediately before the experiment to a solution containing protein (20 μM, 1 ×PBS) to a final concentration of 22 μM mPEG. All thermal melt parameters were the same as listed in the above described experiments.

### L. Sample Preparation for NMR Experiments

Both unconjugated and conjugated samples containing [u-¹⁵N]-Gal3C[T243C], [u-¹⁵N]-Gal3C[T243C,K139I] and [u-¹⁵N]-Gal3C[T243C,K139I,K196I] were concentrated to 15-35 μM in NMR buffer (20 mM sodium phosphate pH 6.9, 30 mM NaCl, 10 mM DTT, 9.5% ²H₂O) at a final volume of 300 mL. For hydrogen-to-deuterium exchange (HDX) experiments with [u-¹⁵N]-Gal3C[T243C]-PEG, a reference [¹⁵N, ¹H]-HSQC spectrum was recorded in buffer containing 9.5% ²H₂O. This sample was then immediately concentrated by rotory evaporation using 2-minute intervals of exposure to vacuum with the chamber heater on and then diluted back to 300 mL with 99.8% ²H₂O. The final NMR sample contained 73.5% ²H₂O, which was confirmed by ¹H NMR. For NMR experiments with samples of Gal3C[T243C]-PEG heated to a temperature required to populate the intermediate state, the protein was prepared in NMR buffer without DTT and without ²H₂O and was heated in a quartz cuvette at a 1° C. min⁻¹ linear ramp using a CD spectrophotometer to monitor the CD signal in real time. Once the sample temperature reached 62° C., the sample temperature was held constant for 3 minutes, and the sample was then cooled down to 30° C. The sample was immediately centrifuged at 17,000 rpm for 15 min to remove any aggregates and the supernatant was immediately prepared for NMR experiments by adding 10 mM DTT and 9.5% ²H₂O to a final volume of 300 mL.

### M. NMR Data Acquisition

NMR data were recorded for temperatures between 30° C. and 55° C., as noted in the main text, with a Broker Avance™ III spectrometer operating at 800 MHz, running Topspin™ version 3.6.3 and equipped with a 5 mm TXI cryoprobe. The temperature was calibrated using a standard sample of 4% methanol in d₄-MeOH. 2D [¹⁵N, ¹H]-HSQC spectra were recorded using a gradient sensitivity-enhanced pulse sequence (hsqcetf3gpsi) with 2048 points in the direct dimension, between 180 to 260 points in the indirect dimension and between 32 to 736 scans per experiment, depending on the final sample concentration to achieve similar signal-to-noise ratios for all data. The ¹⁵N T₁ and T₂ relaxation measurements were acquired with 1D ¹⁵N-edited HSQC-based experiments (hsqct1etf3gpsi3d and hsqct2etf3gpsi3d) with 2048 points and 512 scans per experiment. For T₁ measurements, experiments were recorded with relaxation delays of 0.02, 0.05, 0.1, 0.2, 0.3, 0.4, 0.6, 0.8,1, 1.2 or 1.5 s. For T₂ measurements we used relaxation delays of 17, 34, 51, 68, 85, 102, 136, 170, 204, 254, or 339 ms with a 900 μs inter-pulse delay CPMG.

### N. NMR Data Processing and Spectral Assignment

NMR spectra were processed with TopSpin™ 3.2 and analyzed with NMRFAM-SPARKY version 1.470. Prior to Fourier transformation, the data matrices were zero filled to 1024 (t1)×4096 (t2) complex points and multiplied by 6.0 Hz gaussian and cosine window functions applied to the direct and indirect dimensions, respectively. Spectra recorded for ¹⁵N relaxation measurements were processed using a cosine window function.

Chemical shifts of signals for conjugated at unconjugated Gal3C[T243C] measured at 30° C. were calibrated by global comparison of the spectra and using the signal of backbone amide group of L122. For variable temperature experiments, chemical shifts were calibrated with respect to the reference spectra at 30° C. by subtracting the average shifts of all assigned backbone amides signals and verified with the one of L122.

By comparing the HSQC data of Gal3C[T243C] recorded at 30° C. in the current study with the published chemical shifts of human Gal3C (BMRB entry 4909) and full length human galectin-3 (BMRB entry 19491) also recorded at 30° C., 71% of the backbone amide signals of Gal3C[T243C] could be unambiguously assigned and an additional 17% of the amide signals were tentatively assigned, as indicated in the main text. Given the high degree of similarity of NMR data of Gal3C[T243C] and Gal3C[T243C]-PEG, assignments of the HSQC signals for Gal3C[T243C] were transferred to spectra of Gal3C[T243C]-PEG. The same procedure was followed for assigning signals in spectra of samples of Gal3C[T243C,K139I], Gal3C[T243C,K139I]-PEG, Gal3C[T243C,K139I,K196I] and Gal3C[T243C,K139I,K196]I-PEG.

### O. NMR Data Analysis

Chemical shifts of the backbone amide signals of Gal3C[T243C] and Gal3C[T243C] variants with lysine replacements that were perturbed from PEGylation (CSP (PEG) in Hz) were quantified at each temperature using the following equation:

CSP (PEG)=\sqrt{(ω_{PEG}−ω_{no PEG})_{H}²+(ω_{PEG}−ω_{no PEG})_{N}²}

where ω_{PEG} and ω_{no PEG} are the resonance frequencies of a given amino acid backbone amide for the conjugated and unconjugated protein, respectively, and where H and N written in subscripts outside of the parentheses indicate the amide chemical shifts the chemical shifts for the ¹H and ¹⁵N dimensions, respectively.

The extent of line broadening of NMR signals due to conjugation with PEG (Broadening (PEG)) was quantified using the following equation

$$Broadening ( PEG ) = \frac{( \frac{{I}_{residue}}{{I}_{max}} ) no PEG}{( \frac{{I}_{residue}}{{I}_{max}} ) PEG}$$

where I_{residue} is the signal intensity of a given amino acid backbone amide and Iₘₐₓ is the most intense signal in the spectrum and where no PEG and PEG outside of the parentheses designate the unconjugated and conjugated protein, respectively.

Similar metrics were used to compare the Gal3C[T243C]-PEG that was heated to 62° C. (designated by the subscript heated) to the relative to a freshly prepared Gal3C[T243C]-PEG sample (designated by the subscript FP), both analyzed at 30° C.:

$$CSP ( heating ) = \sqrt{{( {ω}_{heated} - {ω}_{FP} )}_{H}^{2} + {( {ω}_{heated} - {ω}_{FP} )}_{H}^{2}}$$

$$Broadening ( heating ) = \frac{( \frac{{I}_{residue}}{{I}_{max}} ) FP}{( \frac{{I}_{residue}}{{I}_{max}} ) heated}$$

In variable temperature [¹⁵N, ¹H]-HSQC experiments with Gal3C[T243C] and Gal3C[T243C]-PEG, differences in chemical shift perturbations between NMR data measured at 30° C. and a second temperature (40, 50 or 55° C.) were calculated as follows

CSP (T)=\sqrt{(ω_{T}−ω_{30° C.})_{H}²+(ω_{T}−ω_{30° C.})_{N}²} |ΔCSP|=|CSP_{PEG}(T)−CSP_{no PEG}(T)|

where the same notation was used as above equation, T is a given temperature, and |ΔCSP| is the absolute value of the difference in the chemical shift changes between Gal3C[T243C] and Gal3C[T243C]-PEG.

Changes in line broadening observed as a function of temperature (peaks intensity ratio I ratio (T)) and differences in broadening changes (ΔI ratio) were calculated as follows

$$\begin{matrix}I ratio ( T ) = \frac{( \frac{{I}_{residue}}{{I}_{max}} ) T}{( \frac{{I}_{residue}}{{I}_{max}} ) 30 °C} & Δ\end{matrix} I ratio = I {ratio}_{PEG} ( T ) - I {ratio}_{no PEG} ( T )$$

For HDX experiments, a solvent exchange protection factor (HDX protection) was calculated with the following equation

$$HDX protection = \frac{( \frac{{I}_{high 2 H}}{{I}_{low 2 H}} ) residue}{( \frac{{I}_{high 2 H}}{{I}_{low 2 H}} ) max}$$

where I_{high 2H} and I_{low 2H} are the intensity of a given [¹⁵N, ¹H]-HSQC signal for the sample with high and low ²H₂O concentration, respectively, and where residue and max outside of the parentheses designate a given amino acid backbone amide and the most intense signal in the spectrum, respectively.

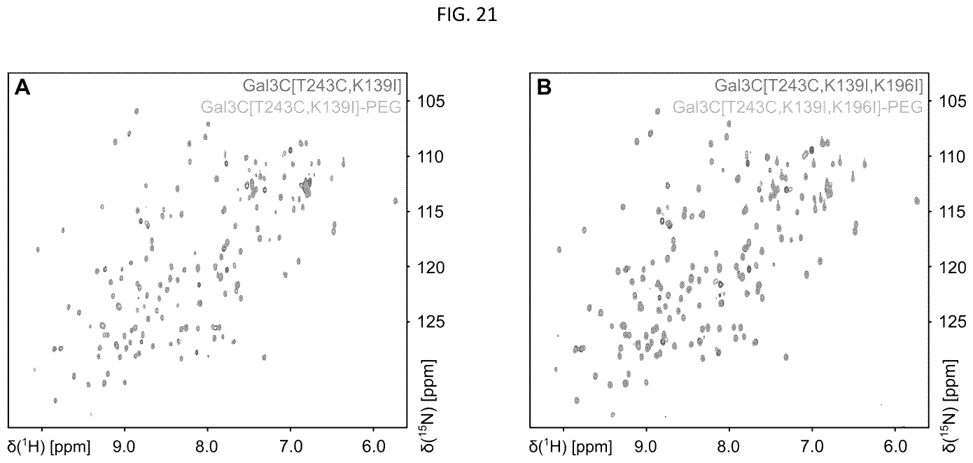

Global ¹⁵N relaxation measurements of Gal3C[T243C] and Gal3C[T243C]-PEG backbone amides at 30° C. were performed by integrating ¹⁵N-edited 1D ¹H signals from 8.50 to 9.95 ppm to select only signals from the backbone amides of the protein folded core (see FIG. 16). The longitudinal relaxation time T₁ and the transverse relaxation time T₂ were obtained by fitting the integrals as a function of relaxation delays with the equations below and using simulated annealing-based optimization from an in-house program (GOSA) to estimate the associated error.

$$I ( t ) = I ( 0 ) + P {e}^{\frac{- t}{{T}_{1}}} \qquad I ( t ) = P {e}^{\frac{- t}{{T}_{2}}}$$

In the above equation I is the signal integral, t is the relaxation delay and P is an amplitude factor.

The rotational correlation times τ_{c} of Gal3C[T243C] and Gal3C[T243C]-PEG was then determined from T₁ and T₂ using the following spectral density function (j(ω)) equations

$$J ( ω ) = \frac{\frac{2}{5} {τ}_{c}}{1 + {( ω {τ}_{c} )}^{2}}$$

$$\frac{1}{{T}_{1}} = d [ 3 J ( {ω}_{N} ) + J ( {ω}_{H} - {ω}_{N} ) + 6 J ( {ω}_{H} + {ω}_{N} ) ] + cJ ( {ω}_{N} )$$

$$\frac{1}{{T}_{2}} = \frac{1}{2} d [ 4 J ( 0 ) + 3 J ( {ω}_{N} ) + J ( {ω}_{H} - {ω}_{N} ) + 6 J ( {ω}_{H} ) + 6 J ( {ω}_{H} + {ω}_{N} ) ] + \frac{2}{3} cJ ( {ω}_{N} )$$

where d and c are the ¹H-¹⁵N dipolar coupling and the ¹⁵N chemical shift anisotropy, respectively, and where ω_{N} and ω_{H} are the Larmor frequencies of ¹⁵N and ¹H, respectively. This analysis follows the model-free approach from Lipari and Szabo ref, which assumes Gal3C contains a relatively rigid backbone and that overall molecular tumbling is the largest contribution to relaxation.

τ_{c} values of Gal3C[T243C] and Gal3C[T243C]-PEG were compared to values published in the literature for proteins of different molar mass. As the τ_{c} values from the literature were determined at 25° C. and the τ_{c} values of Gal3C[T243C] and Gal3C[T243C]-PEG were determined at 30° C., the difference in temperature and corresponding difference in solvent viscosity was accounted for using Stoke's law:

$${τ}_{c} = \frac{4 πη {r}^{3}}{3 kT}$$

### Example 2: Residue-Specific PEGylation of the Carbohydrate Recognition Domain of Galectin-3

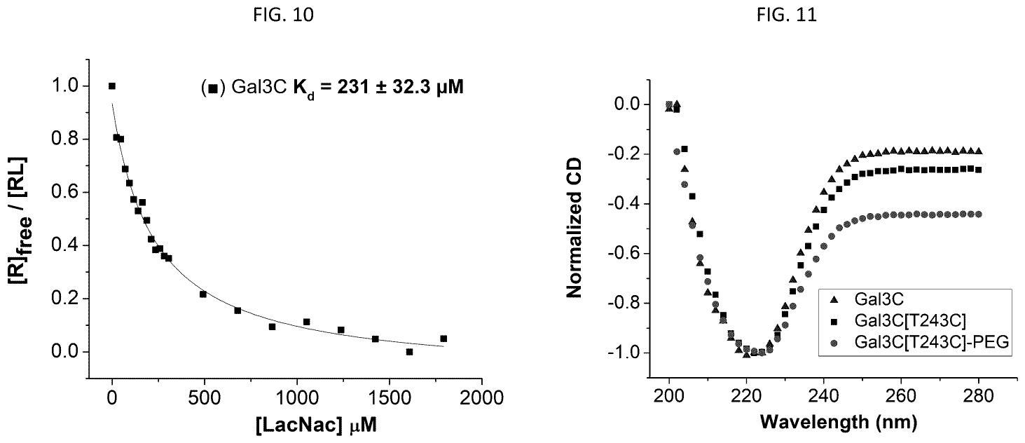

A variant of the carbohydrate recognition domain of human galectin-3 (Gal3C) containing a single extrinsic cysteine at position 243, Gal3C[T243C], was covalently modified with a single 5.4 kDa monomethoxy-poly(ethylene glycol)-maleimide polymer at position 243 (FIG. 1A). Native Gal3C contains only one cysteine, C173, which is buried in the protein hydrophobic core. Attempts to conjugate native Gal3C with PEG resulted in completely unreacted protein, as verified by size exclusion chromatography (see FIG. 8). C243 was selected for conjugation with PEG because it was predicted to be solvent accessible and located on the opposite surface of the Gal3C sugar-binding pocket, and therefore the function of the PEG-conjugated protein was anticipated to be preserved.

FIG. 8 is a representative size exclusion chromatogram of the products of the Gal3C PEGylation reaction after removal of excess PEG-maleimide. The major isolated product is labeled ‘Gal3C’ on the chromatogram.

To produce the reactive PEG, a maleimide group was installed with end-group functionalization of monomethoxy-PEG (mPEG). A maleimide group was chosen as the Michael-acceptor here because it is frequently employed for efficient bioconjugation with cysteines and represented in many PEG-protein conjugates in industrial and clinical applications. Thiol-Michael addition between C243 and the mPEG-maleimide (PEG) produced Gal3C[T243C]-PEG (see FIG. 1B).

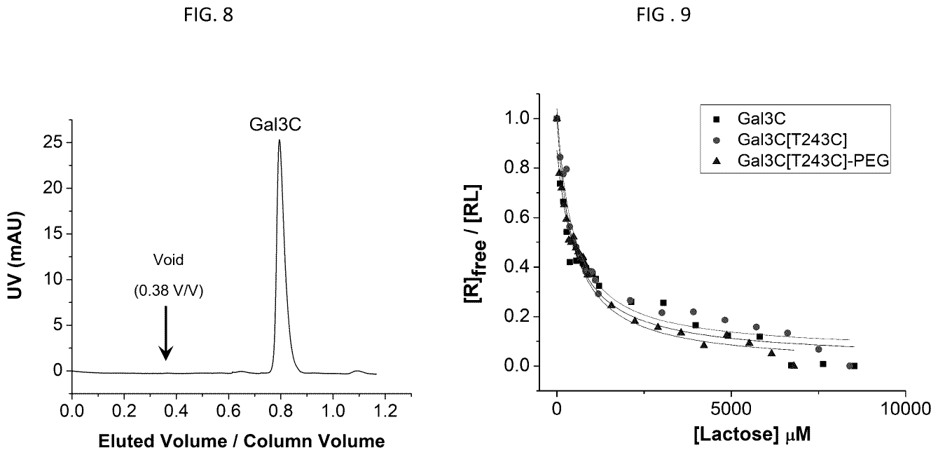

Gal3C[T243C]-PEG was produced and then isolated via two consecutive chromatographic steps that removed excess PEG and unreacted Gal3C[T243C]. Baseline separation of Gal3C-T243C1-PEG from unreacted Gal3C[T243C] was achieved in a final size exclusion chromatography step (see FIG. S1). The resulting monodispersed Gal3C-T243C1-PEG was highly pure, as verified by SDS-PAGE (FIG. 1B). MALDI-TOF mass spectroscopy confirmed that PEG was covalently attached, and the expected 1:1 stoichiometric ratio of PEG:protein was observed (FIG. 7).

FIG. 7 is a representative MALDI-TOF spectrum of a Gal3C[T243C]-PEG sample used for biophysical experiments, showing a single peak near the expected m/z ratio (22.3 kDa). Two superimposed traces (black and red) are from different instrument methods used to generate spectra in two mass ranges of 5-20 kDa (black) and 20-50 kDa (red). No signal was observed in the 5-20 kDa range, as expected.

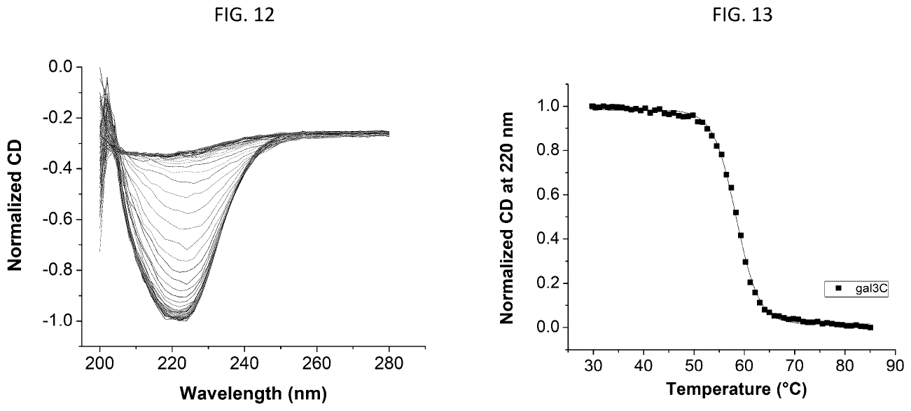

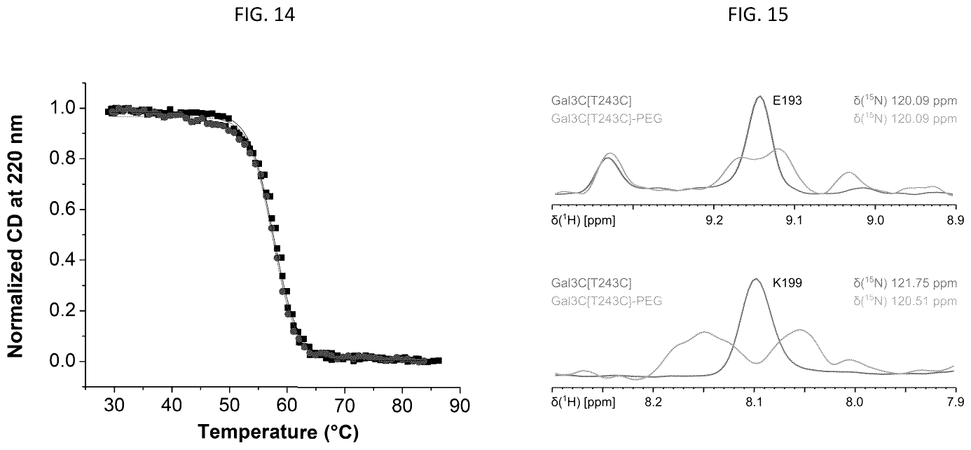

The binding affinity and specificity of Gal3C[T243C]-PEG was compared with Gal3C[T243C] and Gal3C (see FIG. 1C, FIG. 9 and FIG. 10) via an inherent tryptophan fluorescence assay optimized to ensure equilibrium binding conditions. Binding of lactose or LacNac quenches the fluorescence of W181, located adjacent to the bound sugar in the crystal structure of Gal3C, thus permitting direct measurement of ligand K_{d} values. The observed K_{d} for lactose binding to Gal3C[T243C]-PEG was essentially identical to the values measured for Gal3C[T243C] and Gal3C. Further, the binding affinity of LacNac for Gal3C[T243C]-PEG was enhanced 2.4-fold over Gal3C[T243C]. See FIG. 1C and Table S1.

TABLE S1
Equilibrium binding constants (K_{d}) for lactose or LacNac
to Gal3C, Gal3C[T243C], and Gal3C[T243C]-PEG
determined from the tryptophan fluorescence measurements.

| Protein | Ligand | K_{d} (μM)^{a} |
|---|---|---|
| Gal3C[T243C] | Lactose | 413 ± 102 |
| | LacNac | 230 ± 56.5 |
| Gal3C[T243C]-PEG | Lactose | 462 ± 67.7 |
| | LacNac | 95.6 ± 47.6 |
| Gal3C | Lactose | 529 ± 125 |
| | LacNac | 231 ± 32.3 |

^{a}K_{d} is the average K_{d} ± standard deviation determined from 3 replicated experiments.

FIG. 9 shows the equilibrium binding of lactose to Gal3C (black squares), Gal3C[T243C] (red circles) and Gal3C[T243C]-PEG (blue triangles) measured by intrinsic tryptophan fluorescence. The plotted datasets are representative single experiments and the reported K_{d} values are the average K_{d}±standard deviation for each protein, as determined from 3 replicated experiments. See also Table S1, above.

FIG. 10 shows the equilibrium binding of LacNac to Gal3C (black squares) measured by intrinsic tryptophan fluorescence. The plotted dataset is representative of a single experiment and the reported K_{d} value is the average K_{d}±standard deviation, as determined from 3 replicated experiments. See also Table S1.

### Example 3: PEGylation of the Carbohydrate Recognition Domain of Galectin-3 Redirects its Unfolding Pathway

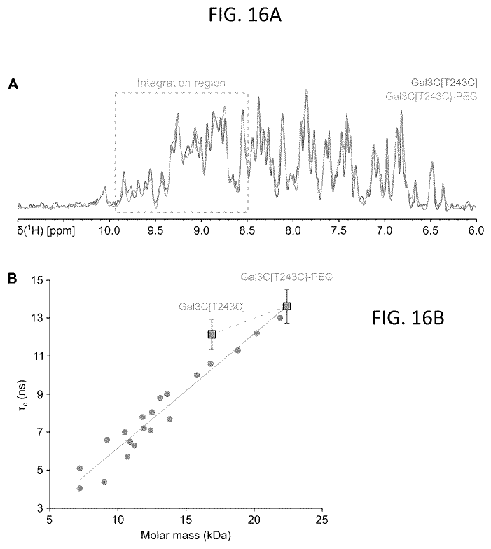

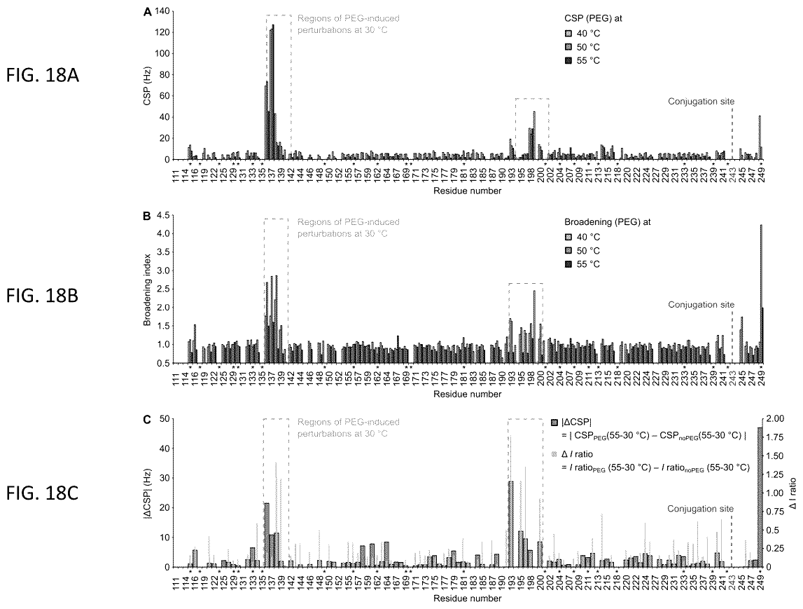

To determine the effect of PEGylation on the global protein fold and thermal stability of Gal3C[T243C] we used circular dichroism (CD) spectroscopy. The CD spectra of Gal3C[T243C] measured at 30° C. are consistent with a folded protein containing mostly b-sheet secondary structure (FIG. 1D) and highly similar to CD spectra of Gal3C measured at the same temperature (FIG. 11). Variable temperature CD experiments of Gal3C[T243C] using a continuous thermal ramp showed a single cooperative unfolding transition with an apparent melting temperature (Tₘ) of 58.1° C. (see FIG. 1D, FIG. 1F, and Table S2), which was indistinguishable from the unfolding behavior of Gal3C (see FIG. S7, FIG. 13, and Table S2) and indicated the T243C amino acid replacement did not alter the thermal stability of the protein. CD spectra of Gal3C[T243C]-PEG measured at 30° C. were highly similar to unconjugated Gal3C[T243C] (see FIG. 1E) and consistent with a folded protein containing highly similar secondary structure.

FIG. 11 shows the superposition of normalized circular dichroism (CD) vs. wavelength plots of Gal3C (black squares), Gal3C[T243C] (red circles) and Gal3C[T243C]-PEG (blue triangles) measured at 30° C. FIG. S7 shows the superposition of normalized circular dichroism (CD) plots of Gal3C measured from 30° C. (region ‘1’) to 90° C. (region ‘2’). The arrow and ‘T’ indicate an increase in temperature for individual experiments plotted in region ‘1’ to region ‘2’. FIG. 13 shows the thermal unfolding of Gal3C (black squares) monitored by normalized CD intensity at 220 nm. The data were fit to a Boltzmann sigmoidal function that showed a single cooperative unfolding event with a fitted melting temperature (Tₘ) of 58.4° C.

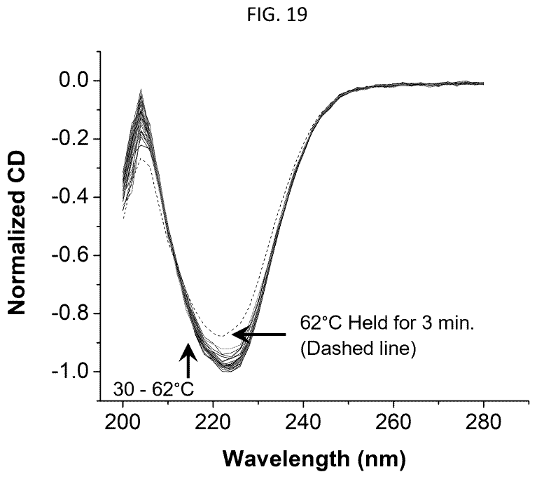

Remarkably, the thermal unfolding of Gal3C[T243C]-PEG was distinct from unconjugated Gal3C[T243C] and showed two separate unfolding transitions with apparent Tₘ values of 56.2° C. and 84.5° C., respectively (FIG. 1E, FIG. 1F, and Table S2). While the lower Tₘ value of Gal3C[T243C]-PEG was similar to Gal3C[T243C], complete unfolding of the conjugated protein was beyond the capabilities of the instrument, and the higher Tₘ value was determined from fitting the data using an estimated value for the third plateau. The first Tₘ observed for Gal3C[T243C]-PEG was determined to be 56.2° C., similar to the measured Tₘ value of Gal3C[T243C]. However, unlike the observations of cooperative unfolding and aggregation of Gal3C[T243C] upon heating, Gal3C[T243C]-PEG showed a transition to an intermediate state (FIG. 1 e and f) and remained soluble at higher temperatures. The CD spectra for Gal3C[T243C]-PEG measured above the first transition temperature show that conjugated Gal3C[T243C]-PEG retains similar b-sheet content at higher temperatures, but the lambda minimum is less than the value observed for Gal3C[T243C] (215 nm versus 225 nm) (FIG. 1E), supporting the formation of an intermediate state at elevated temperatures with subtly different global structure. As a control, we also recorded variable temperature CD experiments with Gal3C[T243C] alone and in the presence of free, non-functionalized PEG added to the solution prior to heating the sample. These data were nearly identical to the data measured for unconjugated Gal3C[T243C] (see FIG. 14). This indicated that direct interactions between Gal3C[T243C] and covalently attached PEG increased the thermal stability of the conjugated protein and drove formation of the intermediate, rather than this observation resulting from a molecular crowding effect from free PEG in solution. We also concluded that PEGylation of Gal3C[T243C] redirects the single step, cooperative and irreversible unfolding pathway of Gal3C, resulting in a two-step unfolding process and formation of an unfolding intermediate that retains significant secondary structure.

TABLE S2
Average melting temperatures from fitting normalized
CD at 220 nm vs. temperature plots of Gal3C, Gal3C[T243C],
and Gal3C[T243C]-PEG.^{a}

| | Tm₁ ± StDev | Tm₂ ± StDev |
|---|---|---|
| Protein | (° C.) | (° C.) |
| Gal3C | 58.8 ± 0.6 | — |
| Gal3C[T243C] | 58.1 ± 0.5 | — |
| Gal3C[T243C]-PEG | 56.2 ± 2.8 | 84.5 ± 2.3 |
| Gal3C[T243C, K139I] | 52.5 ± 0.4 | ND^{b} |
| Gal3C[T243C, K139I]-PEG | 55.1 ± 0.1 | ND^{b} |
| Gal3C[T243C, K139I, K196I] | 60.5 ± 0.6 | ND^{b} |
| Gal3C[T243C, K139I ,K196I]-PEG | 63.5 ± 0.3 | 96.8 ± 1.8 |

^{a}Tm values from 3 replicates were averaged.
^{b}Higher than detectable by instrument (>90° C.)

FIG. 14 shows the thermal unfolding of Gal3C[T243C] (20 μm) in the presence (black squares) and absence (red circles) of 22 μM free PEG-OH measured by normalized CD intensity at 220 nm. The data were fit using a Boltzmann sigmoidal function, and the fitted melting temperatures (Tₘ) for each sample were determined to be 57.7° C. and 57.5° C., respectively.

### Example 4: PEGylation Alters the Gal3C Backbone Local to the Site of Chemical Conjugation

To characterize potential non-covalent interactions between Gal3C[T243C] and covalently attached PEG that redirect the thermal unfolding pathway of Gal3C[T243C]-PEG, a 2-dimensional NMR correlation spectra was recorded with both conjugated and unconjugated Gal3C[T243C]. Previously reported NMR assignments for Gal3C and full-length Gal3 enabled unambiguous assignment of 71% of the backbone amide groups for Gal3C[T243C] and assignment of an additional 17% of the amide groups with some ambiguities. 2D [¹⁵N, ¹H]-heteronuclear single quantum correlation (HSQC) spectra recorded at 30° C. with both [u-¹⁵N] Gal3C[T243C] and [u-¹⁵N] Gal3C[T243C]-PEG were resolved and well dispersed, confirming both unconjugated and conjugated Gal3C[T243C] were folded. See FIG. 2A. The 2D HSQC spectra for unconjugated and conjugated Gal3C[T243C] were highly similar globally (FIG. 2A), demonstrating that PEGylation of Gal3C did not change the overall fold of Gal3C[T243C].

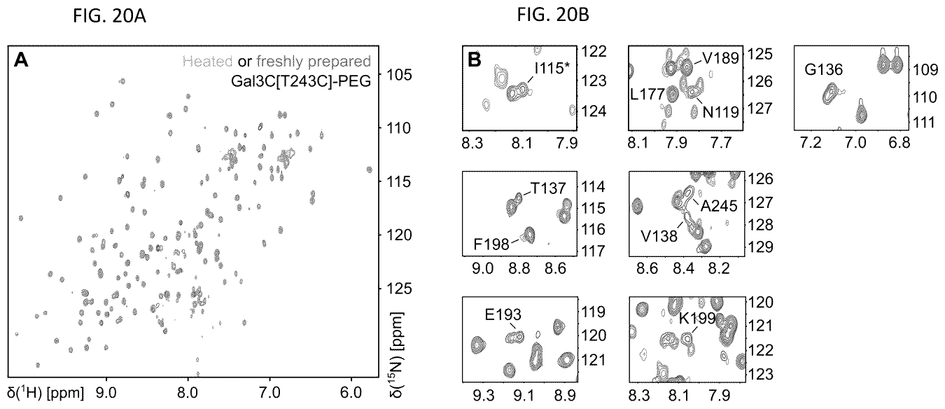

However, comparison of the HSQC spectra for unconjugated and conjugated Gal3C[T243C] revealed several differences for specific amide groups. Significant changes in chemical shifts (>0.05 ppm) and/or changes in line broadening (>25% change in line width) between the HSQC spectra of Gal3C[T243C] and Gal3C[T243C]-PEG were observed for about 12 amide groups predominantly from residues numbered 136 to 141 and 193 to 200. See FIG. 2B, FIG. 2D and Table S3. These perturbations are local to the site of conjugation but not exclusively adjacent to position 243C, encompassing several loops and the ends of two β-strands near the site of conjugation at C243 (see FIG. 2C). Interestingly, while most amide groups showed single NMR peaks, signals for two residues, E193 and K199, were split in the HSQC spectrum of Gal3C[T243C]-PEG (see FIG. 15 and Table S3), suggesting the existence of multiple local conformations at these positions for Gal3C[T243C]-PEG. Because perturbations in the HSQC data could be related to either direct non-covalent interactions between PEG and the Gal3C[T243C] surface or due to an indirect effect of PEGylation on the protein's local conformation and dynamics, further NMR experiments were performed to more thoroughly investigate the nature of these interactions.

FIG. 2 presents the NMR observations of the Gal3C[T243C] structure upon chemical conjugation with PEG at position 243. FIG. 2A shows the superposition of [¹⁵N, ¹H]-HSQC spectra of [u-¹⁵N]-Gal3C[T243C] (blue) and [u-¹⁵N]-Gal3C[T243C]-PEG (orange) measured at 30° C. FIG. 2B shows expanded views of FIG. 2A that were selected to show specific amide signals where significant changes in chemical shift or line widths were observed between Gal3C[T243C] and Gal3C[T243C]-PEG. FIG. 2C is a surface representation of the crystal structure of Gal3C (PDB ID 4R9C). The red sphere indicates the site of chemical conjugation with PEG. Specific amino acids that showed significant changes in NMR signals are shown in stick representation and colored according to the observed difference: chemical shift perturbations (CSP) larger than 10 Hz (blue), line widths broadened by more than 25% (orange) or both chemical shift perturbations and line broadening (green). FIG. 2D is a histogram of the chemical shift perturbations (blue bars) and line broadening (orange bars) observed for Gal3C[T243C]-PEG relative to the unconjugated protein as a function of the residue position. The “*” indicates residues with ambiguous assignments. The green ‘#’ indicates the L114 backbone amide was broadened beyond detection for Gal3C[T243C]-PEG but not for Gal3C[T243C]. Consecutive residues that displayed significant NMR signal perturbations are highlighted by the grey boxes and are mapped on the structure in FIG. 2C.

FIG. 15 shows one-dimensional cross sections from [¹⁵N, ¹H]-HSQC spectra of [u-¹⁵N]-Gal3C[T243C] (blue) and [u-¹⁵N]-Gal3C[T243C]-PEG (orange). The cross sections were selected at the maximum signal intensity in the ¹⁵N dimensions for residues E193 and K199 at the chemical shifts indicated in the plots. Upon chemical conjugation with PEG, signals for E193 and K199 show the presence of splitting or multiple components in the ¹H dimension.

Summary of Gal3C[T243C] amino-acid residues that are perturbated upon conjugation with PEG at 30° C. or upon heating the conjugate to 62° C. before recording NMR spectra at 30° C. Signals with chemical shift differences larger than 10 Hz or line broadening of more than 25% are reported here. See Table S3, below.

TABLE S3
Perturbed Gal3C[T243C] Amino Acid Residues.

| | Secondary | Gal3C[T243C] relative to | Heated relative to freshly |
|---|---|---|---|
| Residue | structure | Gal3C[T243C]-PEG | prepared Gal3C[T243C]-PEG |
| L114 | N-terminus | Broadening beyond detection | — |
| I115* | N-terminus | — | Large signal perturbation |
| V116 | N-terminus | — | Shift and broadening |
| Y118* | β-strand | Broadening | — |
| N119 | β-strand | — | Large signal perturbation |
| G124* | Loop | — | Shift |
| R129* | Loop | — | Broadening |
| I134 | β-strand | Broadening | — |
| G136 | β-strand | Shift and broadening | Broadening |
| T137 | β-strand | Shift and broadening | Large signal perturbation |
| V138 | Loop | Shift and broadening | Large signal perturbation |
| K139 | Loop | Shift and broadening | — |
| N141 | Loop | Shift | — |
| R151 | Loop | — | Shift and broadening |
| F159 | β-strand | — | Shift |
| F163 | β-strand | — | Broadening |
| R169* | β-strand | — | Large signal perturbation |
| L177 | Loop | — | Large signal perturbation |
| V189 | Loop | — | Large signal perturbation |
| E193 | Loop | Shift, broadening, possible splitting | Large signal perturbation |
| G195 | Loop | Shift and broadening | — |
| K196 | Loop | Shift and broadening | Shift |
| F198 | β-strand | Shift and broadening | Broadening |
| K199 | β-strand | Shift, broadening, possible splitting | Shift |
| I200 | β-strand | Shift and broadening | — |
| V213* | Loop | — | Shift and broadening |
| N214 | Loop | Broadening | — |
| I240 | β-strand | Broadening | — |
| L242* | β-strand | Broadening | — |
| A245 | β-strand | Broadening | Large signal perturbation |
| M249* | C-terminus | Shift and broadening | — |

*indicate residues with ambiguous assignments.

### Example 5: Evidence for “Shroud-Like” Polymer-Protein Interactions within PEGylated Gal3C[T243C]

To investigate whether PEG and Gal3C[T243C] behaved more as two independent domains within the conjugated protein or if they interacted more closely, the rotational correlation times (τ_{c}) for Gal3C[T243C] and Gal3C[T243C]-PEG at 30° C. were determined. See FIG. 3A. τ_{c} values were determined from measurements of the ¹⁵N longitudinal (T₁) and transverse (T₂) relaxation times of backbone amides from the well folded cores of conjugated and unconjugated Gal3C[T243C] (see FIG. 3A, FIG. 3B, and FIG. 16A). This approach has been used for determination of rotational correlation times of globular proteins up to ˜25 kDa.

Conjugation of Gal3C[T243C] with PEG increased the rotational correlation time from 10.7 (±0.7) to 12.0 (±0.8) ns. The increased correlation time for Gal3C[T243C]-PEG also was directly observed with a decrease of the transverse relaxation time (T₂) and line broadening observed in ¹⁵N-edited 1-dimensional ¹H NMR spectra of Gal3C[T243C]-PEG. See FIG. 2 and FIG. 16A. The increased rotational correlation time for Gal3C[T243C]-PEG is in line with expectations for a polypeptide chain of increased molecular weight, indicating that the protein and polymer do not behave as two independent subunits connected by a covalent linker. τ_{c} for Gal3C[T243C] and Gal3C[T243C]-PEG was further compared with values of the literature of globular proteins of different molar masses (see FIG. 16B). PEGylation significantly slowed the rotational diffusion of Gal3C[T243C], which appeared to be in line with an estimated rotational correlation time for a single polypeptide chain of the same molecular weight as the Gal3C[T243C]-PEG conjugate (FIG. 16B).

FIG. 16 shows the NMR measurement of the rotational diffusion times of Gal3C[T243C] and Gal3C[T243C]-PEG. FIG. 16A shows superposition of ¹⁵N-edited 1D ¹H spectra of [u-¹⁵N]-Gal3C[T243C] (blue) and [u-¹⁵N]-Gal3C[T243C]-PEG (orange). The region of integration used to determine longitudinal and transverse ¹⁵N relaxation times is highlighted with a grey box and corresponds to signals from well-folded secondary structure. FIG. 16B shows the correlation of rotational correlation times (τ_{c}) with molar masses plotted as blue circles for 20 well-characterized globular proteins from literature data. The grey line is a linear regression of τ_{c} as a function of protein molar mass determined from the literature data. The calculated τ_{c} values for Gal3C[T243C] and Gal3C[T243C]-PEG are plotted as orange boxes with error bars that indicate the 95% confidence interval from the non-linear regression of ¹⁵N relaxation data used to determined τ_{c}. The τ_{c} values for Gal3C[T243C] and Gal3C[T243C]-PEG were temperature-corrected to 25° C. to directly compare with the literature data, as described herein.

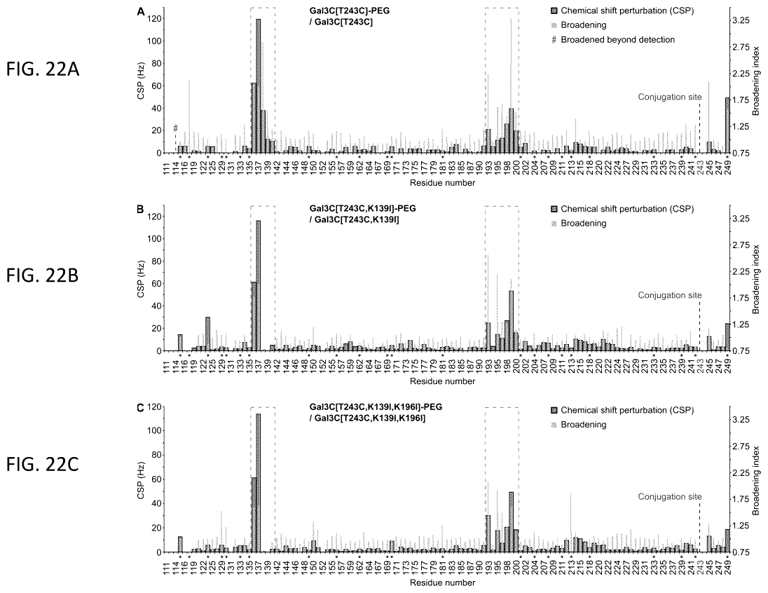

To probe whether the noncovalent interactions between Gal3C[T243C] and PEG persist over longer time scales, we recorded hydrogen-to-deuterium exchange (HDX) NMR data with Gal3C[T243C]-PEG at 30° C. (see FIG. 4A). A [¹⁵N, ¹H]-HSQC spectrum was recorded with [u-¹⁵N] Gal3C[T243C]-PEG, then the same sample was concentrated and diluted with buffer containing 99.9% ²H₂O and a second [¹⁵N, ¹H]-HSQC spectrum was recorded at the same temperature. The final concentration of ²H₂O in the NMR sample was calculated to be 73%. Thus a reduction in the intensity of amide signals proportional to the change in ²H ₂O concentration indicated that an amide group was fully solvent accessible, while a reduction in the NMR signal by less indicated some degree of protection from solvent accessibility, calculated as the HDX ‘protection index’ (see FIG. 17). The regions of Gal3C[T243C]-PEG protected from solvent exchange on the experimental time scale (hours) correlated with the protein's secondary structure (FIG. 17). However, HDX protection did not correlate with the region of amino acids perturbed by PEG conjugation (residues with grey dot representation, FIG. 4A), showing that non-covalent interactions between Gal3C[T243C] and PEG do not persist long enough to prevent bulk solvent molecules from accessing the protein's surface. In line with these observations, ¹H-¹H NOESY experiments recorded with Gal3C[T243C]-PEG at 30° C. failed to show persistent contacts between the protein and polymer (data not shown). Taken together, these results indicate PEG does interact with the surface of Gal3C[T243C] but these interactions were relatively transient. Next, how these interactions may modify the thermal unfolding pathway of the protein conjugate was investigated with variable temperature NMR spectroscopy.

FIG. 17 is a histogram of the relative resistance of Gal3C[T243C]-PEG amide protons to exchange with deuterium in a solution containing 73.5% ²H₂O, indicated as the “HDX protection index”. The values are normalized so that an index of “1.0” indicates the minimal exchange with ²H₂O among the protein backbone amides. The grey schematic at the top represents secondary structural elements in the crystal structure of Gal3C (PDB ID 4R9C). The ‘*’ indicates residues with ambiguous assignments.

### Example 6: Variable Temperature NMR Data Indicate Local PEG-Gal3C Interactions Mediate Formation of the Unfolding Intermediate

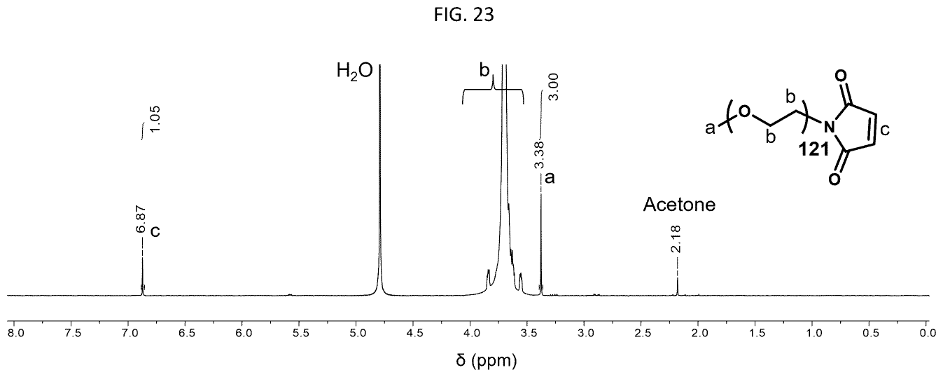

To probe the effects of PEGylation on the behavior of Gal3C[T243C] at higher temperatures, we recorded variable temperature [¹⁵N-¹H]-HSQC spectra of Gal3C[T243C] and Gal3C[T243C]-PEG at 40° C., 50° C. and 55° C., the latter temperature being the highest temperature at which the NMR cryoprobe could be safely operated. For each temperature, chemical shift perturbations and line broadening in NMR data sets of unconjugated and conjugated Gal3C[T243C] was compared to visualize perturbations to the signals that result from PEGylation (see FIG. 18A and FIG. 18B). For all temperatures, we observed chemical shift perturbations and line broadenings at the same residues as described above when comparing NMR data of the conjugated and unconjugated proteins at 30° C. (FIG. 2D). This indicated that perturbations to chemical shifts and line broadenings from PEGylation persisted over the entire range of temperatures studied. PEGylation offered prolonged stability at 55° C. (empirical observation). In addition, the changes in ¹⁵N-¹H HSQC signals that occurred between data recorded at 30° C. and 55° C. were analyzed, for both Gal3C[T243C] and Gal3C[T243C]-PEG. The observed chemical shift perturbations and/or line broadening are globally similar for the protein and the conjugate, but we observed some local differences (see FIG. 18A and FIG. 18B. Interestingly, a number of these differences are clustered on the site of Gal3C[T243C] where PEGylation induced chemical shift perturbations or line broadening at 30° C.

FIG. 18 shows the effect of PEGylation of Gal3C[T243C] measured in comparative NMR spectra from 40° C. to 55° C. FIG. 18A is a histogram plot of chemical shift perturbations (CSP) observed in [¹⁵N, ¹H]-HSQC spectra of Gal3C[T243C]-PEG relative to the unconjugated protein as measured at 40° C. (yellow), 50° C. (orange) and 55° C. (red). The grey box indicates amino acid positions that show chemical shift perturbations upon PEGylation of Gal3C[T243C] measured at 30° C. The site of chemical conjugation with PEG is indicated by the vertical red dashed line, and the ‘*’ indicates residues with ambiguous assignments. FIG. 18B is a histogram plot of observed line broadening in [¹⁵N, ¹H]-HSQC spectra of Gal3C[T243C]-PEG relative to the unconjugated protein as measured at 40° C. (yellow), 50° C. (orange) and or 55° C. (red). Other figure details are similar to FIG. 18A. FIG. 18C is a histogram plot of differences in chemical shift perturbations between [¹⁵N, ¹H]-HSQC spectra of Gal3C[T243C]-PEG relative to Gal3C[T243C] compared between spectra measured at 55° C. and 30° C. Differences in chemical shift perturbations (absolute value, |ΔCSP|) or in peak intensities (Δ I ratio) are displayed as blue and orange bars, respectively. Other figure details are similar to FIG. 18A.

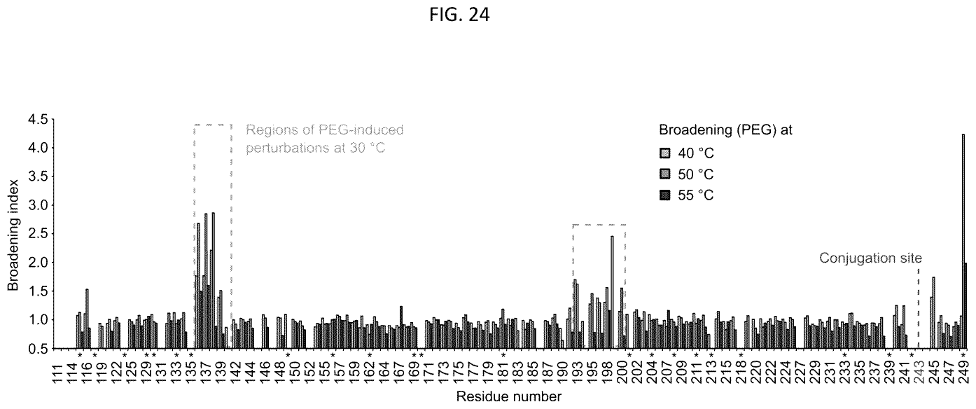

To probe the effect of temperature on Gal3C[T243C]-PEG above 55° C., an NMR sample of Gal3C[T243C]-PEG was heated to 62° C. for 3 minutes before quickly cooling the sample to 30° C. and acquiring a [¹⁵N, ¹H]-HSQC spectrum (FIG. 20). This temperature was above the observed Tₘ of Gal3C[T243C] and was selected to partially populate the intermediate state of the protein while avoiding total irreversible protein unfolding. To prepare this sample, CD spectroscopy was used to monitor the global fold of the protein as a continuous thermal ramp was applied, which showed a slight decrease in intensity but similar lambda minimum value (see FIG. 19). After reaching 62° C., a slight decrease in CD intensity was observed with a similar 1 minimum value as Gal3C[T243C]-PEG at 30° C. The sample was then cooled down to 30° C., and the [¹⁵N-¹H]-HSQC spectrum was immediately recorded.

FIG. 19 shows the superposition of normalized circular dichroism (CD) plots recorded during the preparation of the intermediate state of Gal3C[T243C]-PEG by heating from 30° C. to 62° C. To prepare the sample, a CD spectrum was obtained at each temperature in a continuous thermal ramp from 30 to 62° C. at 1° C. per minute as indicated by the vertical arrow on the plot. Upon reaching 62° C., the sample temperature was held constant for 3 minutes before a final CD spectrum was obtained (black dashed line) and the sample cooled back down to room temperature.

The HSQC spectrum of the sample heated to 62° C. was overall highly similar to the HSQC spectrum of a freshly prepared sample of Gal3C[T243C]-PEG recorded at 30° C. See FIG. 20. This result indicated that the global fold of Gal3C[T243C]-PEG at 30° C. was preserved during the heating and cooling procedure. However, local changes were observed in chemical shifts or line widths for the signals of several backbone amides when comparing the heated and freshly prepared Gal3C[T243C]-PEG. See FIG. 20 and Table 3. These amino acids were almost exclusively located in loops and the ends of β-strands. Interestingly, most of the residues perturbed by heating were the same residues that also displayed chemical shift and/or line width changes upon PEG conjugation (see FIG. 4B, FIG. 4C, and Table S3). This suggests that non-covalent PEG interactions with the Gal3C[T243C] surface induce local changes in the protein structure and dynamics that play a key role in the formation of the unfolding intermediate and ultimately relate to an increase in thermal stability.

FIG. 4 shows mapping Gal3C[T243C]-PEG solvent accessibility and NMR characterization of thermally cycled Gal3C[T243C]-PEG. FIG. 4A shows the solvent accessibility of amide groups of Gal3C[T243C]-PEG determined from HDX experiments mapped onto the crystal structure of Gal3C (PDB ID 4R9C). Amides moderately or highly protected from solvent exchange over a long time scale at 30° C. are colored in pink or purple, respectively, and represented as sticks. The red sphere indicates the site of chemical conjugation with PEG Amino acids shown in a grey dotted space-filling representation indicate residues for which significant changes were observed upon conjugation with PEG in HSQC spectra measured at 30° C. FIG. 4B shows perturbations to amide groups after heating Gal3C[T243C]-PEG to the first transition temperature of 62° C. and then cooling back down to 30° C. Data were measured at 30° C. and compared to a freshly prepared sample of Gal3C[T243C]-PEG measured at the same temperature. Amide signals that showed chemical shift perturbations and/or line broadening are colored orange and represented as sticks. Other presentation details are the same as in FIG. 4A. FIG. 4C is a histogram of the chemical shift perturbations (blue bars) and line broadening (orange bars) observed at 30° C. for Gal3C[T243C]-PEG after heating to 62° C. relative to the native protein conjugate. The * indicate residues with ambiguous assignments. The green ‘#’ indicates amide signals showing very large perturbations. Consecutive residues shown in grey dotted space-filling representation in FIG. 4A and FIG. 4B are highlighted by the grey boxes.

FIG. 20 shows superposition of [¹⁵N, ¹H]-HSQC spectra measured at 30° C. with freshly prepared [u-¹⁵N]-Gal3C[T243C]-PEG (blue) and for the same sample heated to 62° C. for 3 minutes and then cooled back down to 30° C. (orange). FIG. 20A shows the overall [¹⁵N, ¹H]-HSQC spectra and FIG. 20B shows expanded views from FIG. 20A, showing NMR signals that were perturbed upon heating the sample. The ‘*’ indicates the assignment for residue I115 was ambiguous in these data.

### Example 7: Lysines Contribute to the Localization of the Polymer on the Surface of Gal3C[T243C]-PEG but are not Required for Thermal Stabilization

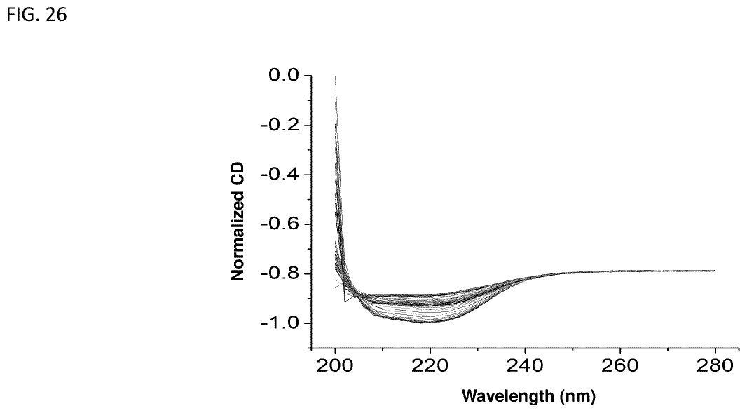

Next, potential determinants that drive the localization of PEG to the region of the surface of Gal3C[T243C] near the site of conjugation were investigated (FIG. 2C), and the potential relationship between PEG localization and the redirection of the thermal unfolding pathway of the PEGylated protein. In several earlier computational studies of PEGylated proteins, results from molecular dynamics simulations proposed that lysines could act as non-covalent ‘anchors’ with PEG on the protein surface through hydrogen bond interactions between the positively charged amino groups on the protein surface and the oxygen atoms in PEG. Three lysines (K139, K196 and K199) formed the perimeter of the region of Gal3C[T243C] for which chemical shift perturbations were observed upon PEGylation. See FIG. 5A. This observation and the earlier computational studies motivated us to replace these specific lysines sequentially with isoleucines, which would not be able to participate as hydrogen bond donors and could potentially disrupt Gal3C[T243C]-PEG interactions. Three Gal3C[T243C] variants were generated: Gal3C[T243C,K139I], Gal3C[T243C,K139I,K196I] and Gal3C[T243C,K139I,K196I,K199I]. Among these, the variants containing one and two lysine replacements could be expressed and purified for NMR and CD experiments (FIG. 5D-I and FIG. 21), but Gal3C[T243C,K139I,K196I,K199I] was not stable enough to produce a PEGylated sample, likely because K199 appears to participate in a salt bridge with D215 located in an adjacent loop. Gal3C[T243C,K139I,K196I,K199I] was, however, subjected to the CD thermal melt assay and exhibited a qualitatively different melting transition as compared to other unmodified variants, indicating that lysines may contribute to stability of the canonical Gal3C native fold (FIG. 5E). The unstable native fold may have prevented generation of stable Gal3C[T243C,K139I,K196I,K199I]-PEG.

Two-dimensional [¹⁵N, ¹H]-HSQC correlation spectra were recorded with [u-¹⁵N] Gal3C[T243C,K139I], [u-¹⁵N] Gal3C[T243C,K139I,K196I] and the corresponding stable isotope-labeled PEGylated variant proteins at 30° C. [¹⁵N, ¹H]-HSQC spectra of the variant proteins were well dispersed, showing the overall fold of the variants was highly similar to Gal3C[T243C], See FIG. 21. The PEGylated variants were also well folded and largely retained the same global fold as Gal3C[T243C], as evidenced by similar [¹⁵N, ¹H]-HSQC correlation spectra. See FIG. 21.

FIG. 21 shows the superposition of [¹⁵N, ¹H]-HSQC spectra measured at 30° C. with unconjugated and PEGylated (FIG. 21A) [u-¹⁵N]-Gal3C[T243C, K139I] and (FIG. 21B) [u-¹⁵N]-Gal3C[T243C, K139I, K196I]. Spectra of the unconjugated and PEGylated variant proteins are colored blue and orange, respectively.

Comparing the HSQC spectra of Gal3C[T243C] with spectra of both variant proteins, we observed subtle differences in the changes of chemical shifts and/or line widths due to PEGylation (see FIG. 22). These differences tended to shift the perturbed region away from K139 and K196 (FIG. 5A and FIG. 5C) and were more widely dispersed across the surface of the proteins, indicating that K139 and K196 participate in localizing PEG. However, replacing these residues with isoleucine was not sufficient to completely abolish PEG interactions with Gal3C[T243C], supporting the idea that other residues on the surface of Gal3C also interact with PEG.

FIG. 22 is a histograms of the chemical shift perturbations (blue bars) and line broadening (orange bars) observed for the residues of PEGylated Gal3C variants relative to their unconjugated counterparts. FIG. 22A: Gal3C[T243C]-PEG relative to Gal3C[T243C] as shown in FIG. 2D; FIG. 22B: Gal3C[T243C,K139I]-PEG relative to Gal3C[T243C,K139I] and FIG. 22C: Gal3C[T243C,K139I,K196I]-PEG relative to Gal3C[T243C,K139I,K196I]. In FIG. 22B and FIG. 22C, the chemical shift perturbations and line broadening values are not displayed for residues 114, 116, 118, 138 and 139 as compared to FIG. 22A because it was not possible to transfer their assignments to the NMR spectra of unconjugated and conjugated Gal3C[T243C,K139I] and Gal3C[T243C,K139I,K196I]-PEG. The ‘*’ indicate residues with ambiguous assignments. The green ‘#’ in FIG. 22A indicates the L114 backbone amide which was broadened beyond detection for Gal3C[T243C]-PEG but not for Gal3C[T243C]. Residues of Gal3C[T243C]-PEG that displayed the most significant NMR signal perturbations relative to Gal3C[T243C] are highlighted by the grey boxes in all three histograms for reference.

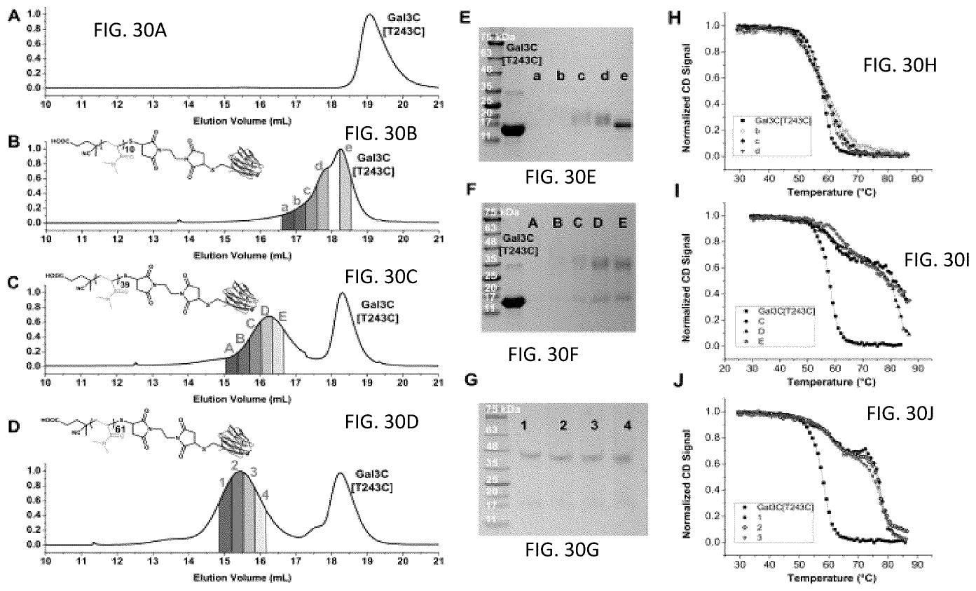

To probe the impact of PEGylation on the thermal unfolding of the Gal3C[T243C] lysine-to-isoleucine variants, variable temperature CD spectra were recorded for Gal3C[T243C,K139I], Gal3C[T243C,K139I,K196I] and the corresponding PEGylated proteins (FIG. 5 and Table S2). Similar to the unfolding behavior of Gal3C[T243C], both Gal3C[T243C,K139I] and Gal3C[T243C,K139I,K196I] displayed cooperative one step unfolding (see FIG. 5D, FIG. 5F, FIG. 5G, and FIG. 5I). Compared with Gal3C[T243C], Gal3C[T243C,K139I] showed a lower Tₘ value of 52.5° C., and Gal3C[T243C,K139I,K196I] exhibited a slightly higher Tₘ value of 60.5° C. (see Table S2). Gal3C[T243C,K139I,K196I,K199I] displayed a significantly altered unfolding profile and a precise Tₘ value could not be determined due to the significantly decreased sample stability (FIG. 26).

With Gal3C[T243C,K139I]-PEG and Gal3C[T243C,K139I,K196I]-PEG (FIG. 5E, FIG. 5F, FIG., 5H, and FIG. 5I), it was observed that lysine-to-isoleucine substitutions did not abolish the formation of the unfolding intermediate nor the thermal stabilization of the protein upon PEGylation. However, compared to Gal3C[T243C], the unfolding intermediate of Gal3C[T243C,K139I]-PEG was further extended to even higher temperatures (see FIG. 5E and FIG. 5F). The thermal unfolding profile of Gal3C[T243C,K139I,K196I]-PEG was more similar to Gal3C[T243C] but showed the presence of multiple states, based on the appearance of multiple plateaus (see FIG. 5H and FIG. 5I). These changes appear to correlate with changes in the localization of PEG on the surface of the conjugated protein and suggest that modulating the localization of PEG on the protein surface can result in changes to thermal unfolding profiles of the conjugated proteins.

FIG. 5 shows the impact of lysine residues on the spatial localization of PEG and corresponding changes to the thermal unfolding profile of PEGylated Gal3C. FIG. 5A, FIG. 5B and FIG. 5C are ribbon representations of Gal3C. Green colors indicate significant chemical shift perturbations or line broadening for backbone amide signals for FIG. 5A Gal3C[T243C]-PEG, FIG. 5B Gal3C[T243C,K139I]-PEG, and FIG. 5C Gal3C[T243C,K139I,K196I]-PEG upon PEGylation of the proteins. The red sphere indicates the site of chemical conjugation with PEG. For FIG. 5B and FIG. 5C, lysines shown in black stick representation have been replaced by isoleucine in the corresponding PEGylated variant proteins. FIG. 5D, FIG. 5E, FIG. 5F, FIG. 5G, FIG. 5H, and FIG. 5I show thermal unfolding of Gal3C and PEGylated Gal3C variants monitored by variable temperature CD. Superposition of normalized CD vs. wavelength plots of measured from 30° C. to 90° C. for FIG. 5D Gal3C[T243C,K139I] and FIG. 5E Gal3C[T243C,K139I]-PEG. FIG. 5F shows thermal unfolding of Gal3C[T243C,K139I] (black squares) and Gal3C[T243C,K139I]-PEG (red circles) monitored by CD at 224 nm. FIG. 5G and FIG. 5H shows superposition of normalized CD vs. wavelength plots measured from 30° C. to 90° C. of FIG. 5G Gal3C[T243C,K139I,K196I] and FIG. 5H Gal3C[T243C,K139I,K196I]-PEG. FIG. 5F shows thermal unfolding of Gal3C[T243C,K139I,K196I] (black squares) and Gal3C[T243C,K139I,K196I]-PEG (red circles) monitored by CD at 224 nm.

### Example 8: Methods for Examples 9-16

### A. Materials

Poly(ethylene glycol) mono methyl ether (mPEG, Mn=5k g/mol) was purchased from Sigma Aldrich. Mesyl chloride (Ms, >99%) was purchased from TCI America. Dimethylformamide (DMF, 99.8%), dichloromethane (DCM), chloroform (CHCl₃) and triethyl amine (TEA, 99%), anhydrous sodium acetate (NaAc) and 29% NH₄OH were purchased from Fisher Chemical. Sodium phosphate monobasic monohydrate (NaH₂PO₄·H₂O), sodium phosphate dibasic anhydrous (Na₂HPO₄) and sodium chloride were purchased from Fisher Chemical. Dithiothreitol (DTT, >99%) was purchased from GoldBio. Hydrazine monohydrate (65% N₂H₄, 98% reagent grade) was from Sigma. Ethylene diamine (99%), maleic anhydride (99%), and acetic anhydride (99%) were purchased from Oakwood Chemical. The 4-(((2-Carboxyethyl)thio)carbonothioyl)thio)-4-cyanopentanoic acid) CTA (95%) was purchased from Boron Molecular. N,N-dimethylacrylamide (DMA, 99%) was from Sigma-Aldrich (stabilized). Poly(ethylene glycol) methacrylate (PEGMA) was from Sigma-Aldrich. DMA and PEGMA were filtered over basic alumina plugs before use. Azobisisobutyronitrile (AIBN, 98%) was purchased from Sigma Aldrich and recrystallized from methanol before use.

### B. Polymer Synthesis

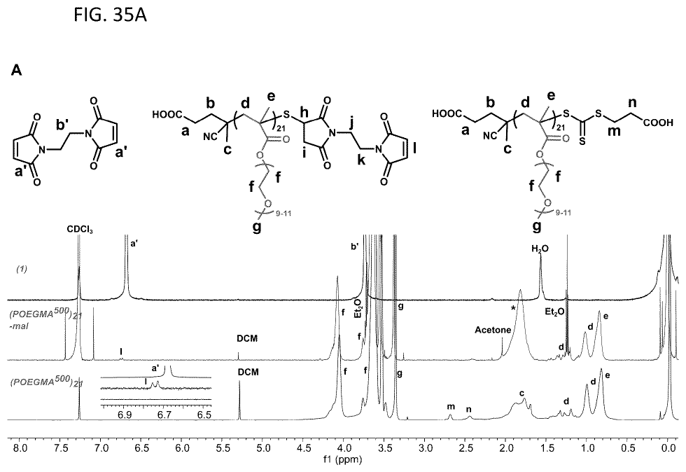

Polymer functionalization is shown in FIG. 32. Before polymerization, DMA and OEGMA monomers and dioxane solvent were purified by passing over a plug of basic alumina. The solvent volume was selected to dissolve monomers at a concentration of 2M. Solid CTA (4-(((2-carboxyethyl)thio)carbonothioyl)thio)-4-cyanopentanoic acid), 6×10⁻⁴ mol, 200-300 mg, 1 equiv.), recrystallized AIBN initiator (6×10⁻⁵ mol, 11 mg, 0.10 equiv.), DMF internal standard (300 uL), and monomer were dissolved in dioxane in a 50 mL Schlenk flask. Flasks were sealed and purged for 30 minutes with N₂ at room temperature before initiating polymerization in a 70° C. oil bath. Polymerization was monitored with GPC and ¹H NMR spectroscopy. When target conversion was reached, the flask was removed from heat and exposed to oxygen to quench the reaction. Polymers were isolated by 3× precipitation in 10 volume equivalents of cold ether. The solid (PDMA) or oil (POEGMA) polymers were isolated and dried under high vacuum overnight before final characterization. The specific monomer amounts, polymerization times, monomer conversion %, and yields are listed for each of the 5 polymers as follows: POEGMA⁵⁰⁰21 (0.0225 mol, 10.5 mL, 19.7 equiv./180 min/55%/6.03 g). POEGMA³⁰⁰20 (0.0224 mol, 6.45 mL, 19.7 equiv./160 min/54%/2.50 g). PDMA₁₀ (0.012 mol, 1.21 mL, 25 equiv./185 min/32%/0.180 g). PDMA39 (0.026 mol, 2.68 mL, 40 equiv./165 min/93%/2.09 g). PDMA62 (0.062 mol, 6.40 mL, 95 equiv/115 min/65%/3.00 g).

C. Synthesis of (1) bis-maleimide [1,1′-(ethane-1,2-diyl)bis(1H-pyrrole-2,5-dione)]:

To generate the bis-amide intermediate, a literature procedure from Sava, M. et al. was adapted. 1 Ethylene diamine (1 equiv., 10 mmol, 0.60 g, 0.67 mL) in 25 mL of acetone was added dropwise to a solution of maleic anhydride (2.5 equiv., 28 mmol, 2.78 g) in 20 mL of acetone in a 100 mL flask. White precipitate was observed immediately upon addition of the amine After 1.5 h, the reaction was heated to 35° C. and stirred for 30 more minutes. The flask was then cooled to room temperature and the solid was filtered out and dried under vacuum. The filtrate was stirred for 2 more days and more solid was collected. The bis-amide intermediate was confirmed by ¹H NMR and the solid used as-is in the next reaction.

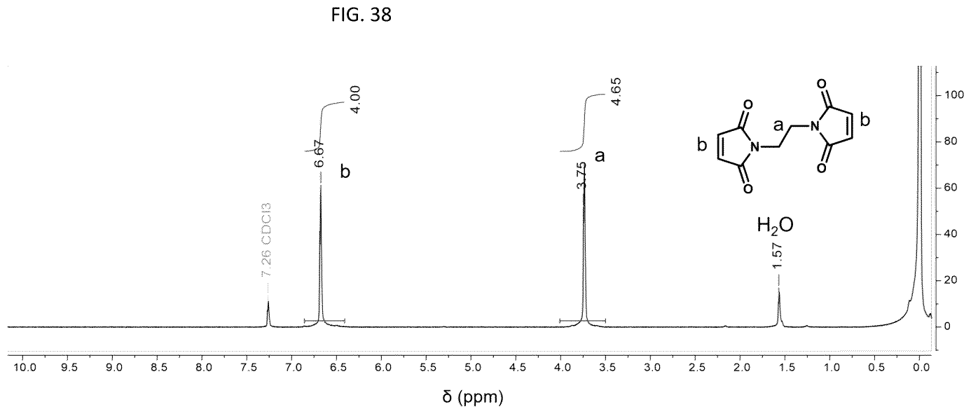

To ring close the product and generate (1), a procedure from Cava, M. P., et al. was adapted.² The crude bis-maleamic acid (1 equiv., 7.93 mmol, 2.03 g) was dissolved in 10 mL of acetic anhydride in a 25 mL round bottom flask. Sodium acetate (2.2 equiv., 17.4 mmol, 1.43 g) was added to the flask and was sparingly soluble. The flask was then sealed, purged, and stirred at 100° C. for 35 minutes until one major product spot was visible on TLC. The reaction was quenched with 50 mL of cold water and an opaque, dull precipitate was observed and filtered off for further purification. The aqueous solution was then extracted 3× with ethyl acetate and the ethyl acetate layer and the previously precipitated solid were combined and purified via silica chromatography with 1 L of 1:1 hexane: ethyl acetate. Final recovery of (1) was 720 mg/3 mmol/30% yield. The ¹H NMR of (1) is shown in FIG. 38.

### D. NMR Spectroscopy of Small Molecules and Polymers

All ¹H NMR samples were analyzed using a 500 MHz Varian or 400 MHz Bruker instrument and prepared in D₂O or CDCl₃ with approximate concentrations of: 5 mg/mL for all small molecules or 10-15 mg/mL for all polymers.

### E. Polymer End Group Functionalization

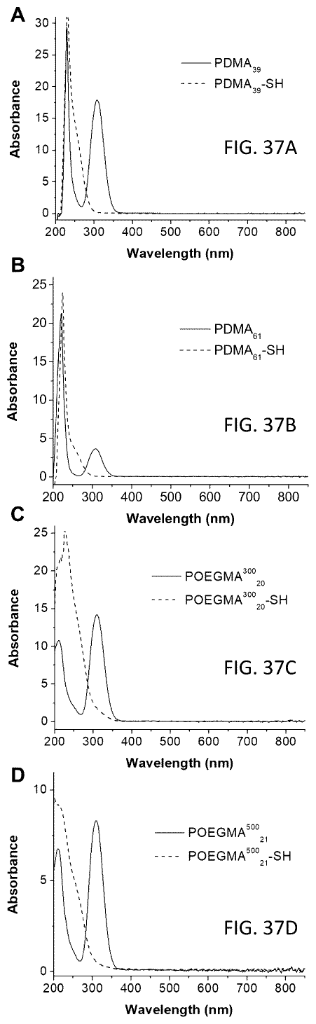

Step 1: Aminolysis. The procedure from Shen et al. ³, was followed with some changes to carry out the CTA cleavage in PBS buffer. Generally, polymers were dissolved at a concentration of 100 mg/mL in 1-2 mL of 1×PBS. Hydrazine monohydrate (5 equiv.) was added to the reaction and it was immediately sealed and purged with N2 before stirring. The reactions were monitored visually for disappearance of yellow CTA color and via UV-Vis with a Nanodrop spectrophotometer at 315 nm. In PBS, quantitative trithiocarbonate cleavage (FIG. 37) took 1-2 hours. The reaction was then cleaned up via desalting using manufacturer protocols for either an AKTA Pure system equipped with a Cytiva 5 mL HiTrap desalting column (1.5 mL max loading volume) or a Cytiva PD-10 gravity desalting column with Sephadex G25 resin (1.75-2.5 mL loading volume). Collected fractions were monitored via UV-vis. A 90% recovery of polymer was assumed for each desalted sample to calculate final polymer yield.

Step 2: Thiol-Michael addition of bis-maleimide (1) Immediately following aminolysis cleanup, 1- 2 mL portions of the polymer-SH in buffer were reacted with bis-maleimide (1) (pre-dissolved in DMF for final reaction concentration of 25% DMF) and triethylamine (1 equiv.). All solutions were purged with N₂ before and immediately after addition of reagents into sealed 10 mL flasks. The total reaction time was 4 h at room temperature. Samples were again desalted to purify while tracking absorbance of fractions at 280 nm. Fractions were pooled and the final concentration of polymer was calculated assuming 90% recovery, per manufacturer standards. Maleimide/DMF could be seen eluting after the polymer via UV-vis measurement of fractions at 280 nm. To quantify conversion, aliquots of the polymer solution were dried down to solid buffer salts and polymer and redissolved in CDCl₃. The conversion was calculated using POEGMA ¹H NMR spectra. Integration of the maleimide 6.7 ppm peaks against the sharp 3.35 ppm methyl peak resulted in 12-21% conversion. Stocks were either used fresh for conjugation or flash frozen.

### F. Grafting-to Conjugation and Purification

Conjugates were prepared with stock solutions of polymer targeting 10 equiv. of polymer:protein. Aliquots of polymer stock of 0.3-0.5 mL were used to obtain the target equivalents. A range of 5-10 mg of Gal3C[T243C] (>100 nM) was reacted with the polymer stock in 1×PBS buffer, at room temperature, while stirring gently for 4 h.

Lactose affinity chromatography was used to remove unreacted polymer and any unfolded protein from the reaction. The reaction was first quenched with 10 mM DTT and bound to lactose-agarose resin (10 mg protein: 1 mL resin) by rotating at 4° C. for 2 h. The mixture was then placed in a mini-column, washed with 10CV of cold PBS (up to 3 mL), and eluted with 30 mM lactose in PBS in single 0.25-0.5 mL aliquots-each following a 15-minute incubation time where the column was just capped.

Size exclusion chromatography (SEC) was performed as a final purification step using an AKTA system with a Superdex 200 Increase 10/300 GL column (0.5 mL/min, inject at 0.05 CV, isocratic elution for 1.1 CV). Protein-polymer conjugates were analyzed using SDS-PAGE with a reducing tris tricine buffer system. Conjugate fractions were collected in sequential 0.330 mL volumes from 0.55-0.86 CV for screening.

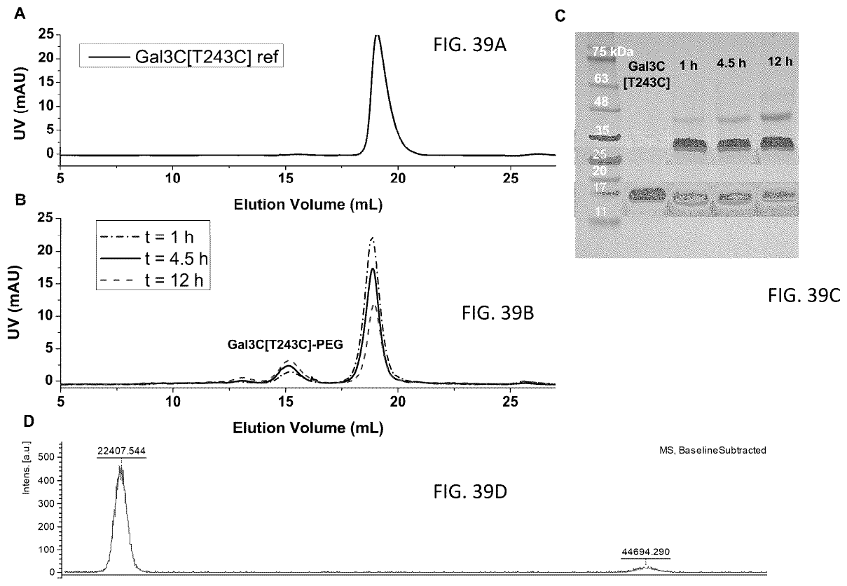

For the control Gal3C[T243C] reaction with 10 equiv. of PEG-mal (FIG. 39), 8.1 mg (10 equiv.) of PEG was reacted with 1.29 mg (1 equiv.) of protein at room temperature in 1.5 mL PBS. At the target time points, 0.5 mL aliquots were selected for SEC and SDS-PAGE, without further purification of the conjugate (from excess PEG-mal) performed. The procedure for synthesis of PEG-mal from purchased mono-methoxy PEG was reported previously.⁴

### G. Protein Production

The plasmid containing Gal3C[T243C] with a 6 ×C-terminal His-tag was transformed into E. coli Origami B (DE3) competent cells. Protein expression was carried out using a previously reported protocol.⁴ Unlabeled Gal3C[T243C] was expressed in 2×TY media. [u-¹⁵N]-Gal3C[T243C] was expressed in M9 minimal media containing 1 g/L ¹⁵N ammonium chloride (Cambridge Isotope Labs).

Gal3C[T243C] purification followed previously reported procedures.⁴ In brief, thawed cell pellets were resuspended in cold PBS, 10 mM DTT and protease inhibitor and lysed three passages through a cell disruptor (Pressure Biosciences) at 25 kpsi. The lysate was clarified by centrifugation, filtered with 0.45 μM GF+PES syringe filters, and mixed with cobalt IMAC resin at the ratio of 0.5 mL resin per 2 L of original culture volume in conical tubes. The filtered lysate and resin were rotated at 4° C. for 20-30 minutes then washed with 1×PBS (50 mL), 1×PBS and 500 mM NaCl (50 mL), and 1×PBS and 5 mM imidazole (30 mL). The protein was eluted with 5 mL of 1×PBS and 75 mM imidazole after first allowing the buffer to enter the resin bed and incubated for 15 minutes. 0.5 mL fractions were collected and highest concentration fractions pooled.

### H. Gel Permeation Chromatography:

Number-averaged molecular weights (Mn) and dispersity were obtained by performing GPC in N,N-dimethylacetamide (DMAC) with 50 mM LiCl at 50° C. with a flow rate of 1.0 mL/min and using multi-angle light scattering detection (Agilent Infinity II isocratic pump, degasser and autosampler and ViscoGel I-series 5 μm guard column, Malvern I-MBLMW and IMBHMW 3078 columns with an exclusion limit of 20,000 g/mol and 1.0×10\hat{ }7 g/mol, respectively). Conventional calibration with poly(methyl methacrylate) standards was employed for POEGMA samples. The detection sources were a Wyatt Optilab T-rEX refractive index detector operating at 658 nm and a Wyatt miniDAWN Treos light scattering detector operating at 659 nm. Molecular weights and molecular ions were calculated using the Wyatt ASTRA software.

### I. R Sample Preparation, Data Acquisition and Analysis

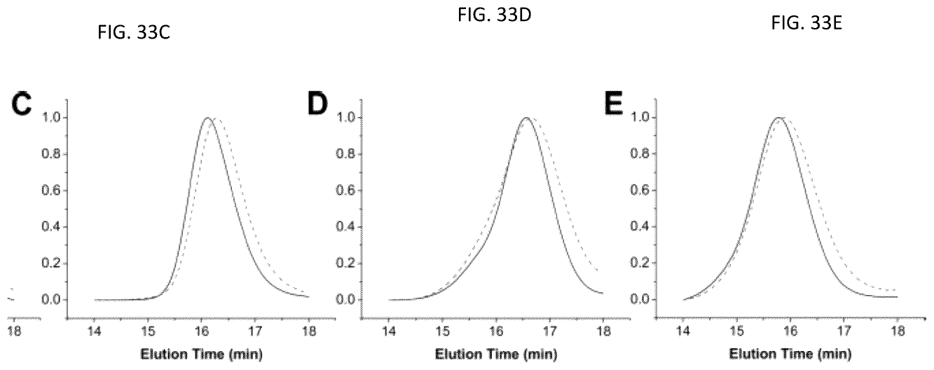

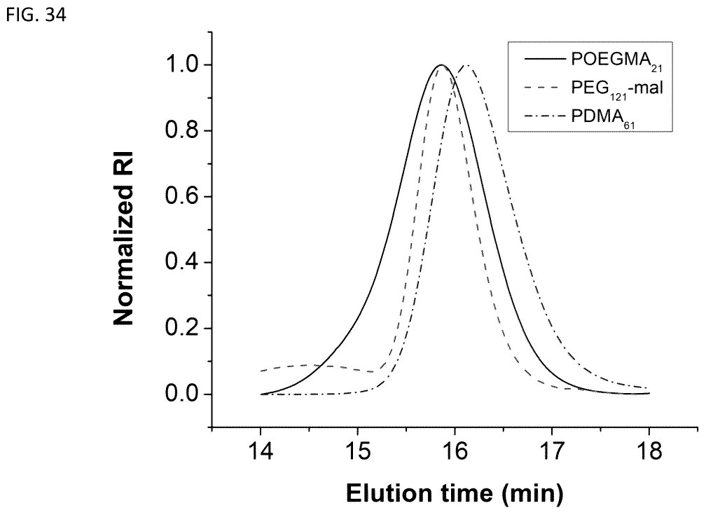

NMR sample preparation, data acquisition and processing followed previously described protocols to be consistent with earlier work.⁴ NMR samples were concentrated to 20 μM for [u-¹⁵N]-Gal3C[T243C]-P(OEGMA⁵⁰⁰)21 and 40 μM for [u-¹⁵N]-Gal3C[T243C]-PDMA₆₁. The POEGMA conjugated NMR sample was prepared by pooling fractions from the top of the conjugate peak ranging from 15.05-15.71 mL (Fraction C and immediately adjacent fractions), as shown in FIG. 31C. The PDMA conjugated NMR sample was prepared by pooling fractions from 15.19-15.85 mL (Fractions 2 and 3 in FIG. 30D). NMR samples were exchanged into NMR buffer (20 mM sodium phosphate pH 6.9, 30 mM NaCl) via a final SEC purification step and ²H₂O was added to the final NMR sample to a final 9.5% v/v ratio.

NMR data were recorded with a Bruker Avance III spectrometer operating at 800 MHz, running Topspin version 3.6.3 and equipped with a 5 mm TXI cryoprobe. The temperature was calibrated using a standard sample of 4% methanol in d4-MeOH. 2D [¹⁵N, ¹H]-HSQC spectra were recorded using a gradient sensitivity-enhanced pulse sequence (hsqcetf3gpsi) with 2048 points in the direct dimension, 180 points in the indirect dimension and 736 or 400 scans for [u-¹⁵ N]-Gal3C[T243C]-P(OEGMA⁵⁰⁰)21 and [u-¹⁵ N]-Gal3C[T243C]-PDMA₆₁, respectively.

NMR spectra were processed with TopSpin 3.2 and analyzed with NMRFAM-SPARKY version 1.470. Prior to Fourier transformation, the data matrices were zero filled to 1024 (t1)×4096 (t2) complex points and multiplied by 6.0 Hz gaussian and cosine window functions applied to the direct and indirect dimensions, respectively. Chemical shift perturbations and broadening indices were calculated by comparing conjugate spectra to [u-¹⁵N]-Gal3C[T243C]. Chemical shift perturbations of the backbone amide signals were quantified using the following equation:

CSP(conjugate)=\sqrt{(ω_{conjugate}−ω_{no conjugate})_{H}²+(ω_{conjugate}−ω_{no conjugate})_{H}²}

where ω_{conjugate} and ω_{no conjugate} are the resonance frequencies of a given amino acid backbone amide for the conjugated and unconjugated protein, respectively, and where H and N written in subscripts outside of the parentheses indicate the amide chemical shifts for the ¹H and ¹⁵N dimensions, respectively.

The extent of line broadening of NMR signals due to conjugation (Broadening (conjugate)) was quantified using the following equation

$$Broadening ( conjugate ) = \frac{( \frac{{I}_{residue}}{{I}_{max}} ) no conjugate}{( \frac{{I}_{residue}}{{I}_{max}} ) conjugate}$$

where I_{residue} is the signal intensity of a given amino acid backbone amide and I_{2′3} is the most intense signal in the spectrum and where no conjugate and conjugate outside of the parentheses designate the unconjugated and conjugated protein, respectively.

### J. Circular Dichorism (CD) Spectroscopy Thermal Melting and Analysis

CD spectroscopic data were recorded with an Applied Photophysics Chirascan spectrophotometer equipped with a Quantum Northwestern Peltier temperature control device and operating with Chirascan v4.7.0 and Pro-Data Viewer software, following procedures used in our previous study.⁴ Spectra were background subtracted against the sample buffer. For thermal unfolding experiments, a linear thermal ramp was applied from 30° C. to 90° C. at 1° C. per minute with samples concentrated to 10 μM in PBS. All spectra were measured in triplicate, and data were normalized to account for slight differences in protein concentration.

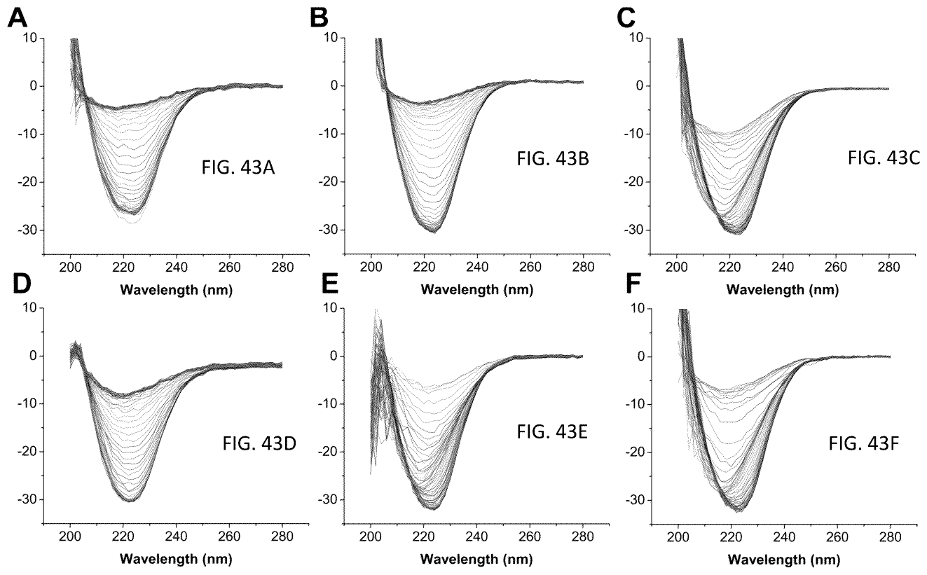

Full wavelength (200-280 nm) CD spectra for each construct (FIG. 43) were used to calculate a wavelength of greatest change (222 or 224 nm) to create single wavelength CD vs. temperature plots (FIG. 30H-J and FIG. 31F-G). The single wavelength CD signal vs. temperature plot of Gal3C[T243C] used data acquired at 220 nm, which was the wavelength of greatest change for the unmodified protein. To calculate melting temperatures, the normalized data were fit to a Boltzmann sigmoidal function in Origin v8.5. For the data fitting, no parameters were fixed, and the ‘x’ parameter defined as the center of the data was interpreted as the melting temperature (Tₘ).

### K. Fluorescence Equilibrium Binding Assay

Equilibrium binding data acquisition and fitting were done according to previous work.⁴ Intrinsic tryptophan fluorescence was monitored using a Cary Eclipse Fluorescence Spectrophotometer (Agilent) operating with Cary Eclipse WinFLR software version 1.2 in ‘Scan Mode’ with an excitation wavelength of 280 nm, acquisition range from 290 nm to 440 nm and photomultiplier tube sensitivity set to ‘medium’. Samples of Gal3C[T243C] conjugates were prepared by pooling SEC fractions, as was done to prepare NMR samples (above), and diluting to 5-6 μM in PBS. Samples were titrated with the N-acetyllactosamine (LacNac) ligand until equilibrium binding conditions were established of at least 10 times the theoretical K_{D}, recording at least 20 data points per titration.

“Hill 1 equation” in Origin v8.5 software, describing reversible binding of a protein to a ligand, was used to fit the normalized equilibrium binding data. Fixed parameters included the Start and End values, which were set to 1 and 0, respectively. Replicated experiments with a fit error of ≤30% were used to the calculate the average K±standard deviation with a total of 3 independent replicated experiments.

### Example 9: Polymer Synthesis and Characterization

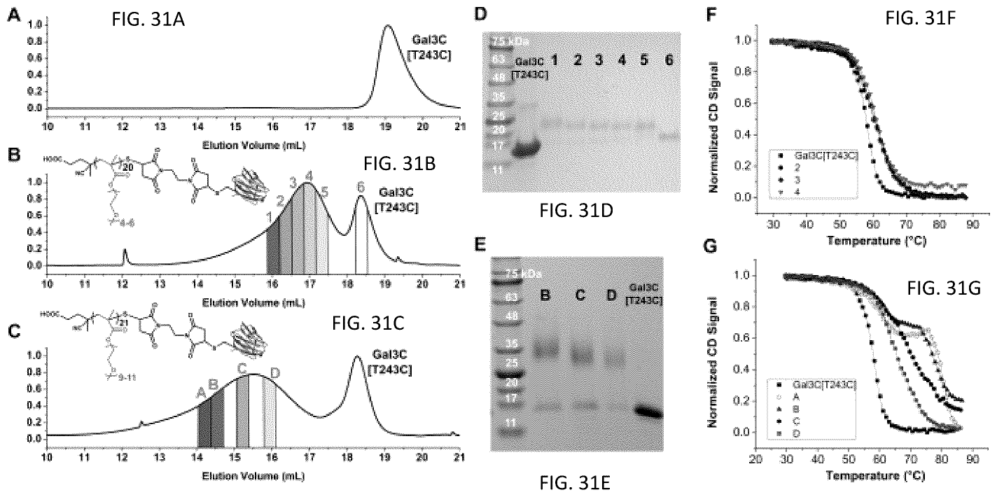

Protein—polymer conjugates were generated with a grafting-to scheme in which a complete polymer was covalently attached to Gal3C[T243C] using thiol-Michael addition (FIG. 27A). The grafting-to method was selected to ensure mild conjugation conditions that preserved Gal3C structure and function, despite the drawback of sterically limited conversion. RAFT polymerization (FIG. 32A) was used to generate five different polymers for conjugation with the following three monomers: dimethylacrylamide (DMA) and two molecular weights of oligoethylene glycol methacrylate (OEGMA³⁰⁰ and OEGMA⁵⁰⁰), which have structures shown in FIG. 32C. The ethylene glycol (EG) monomer of PEG is also included for comparison in FIG. 27B.

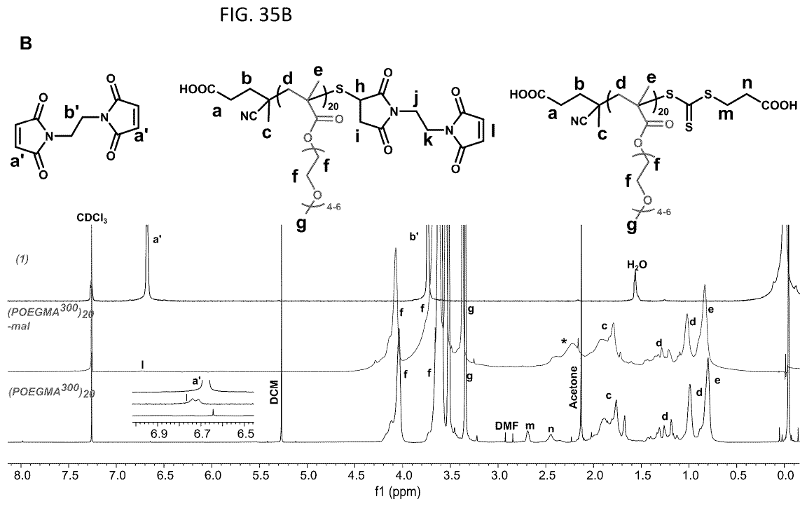

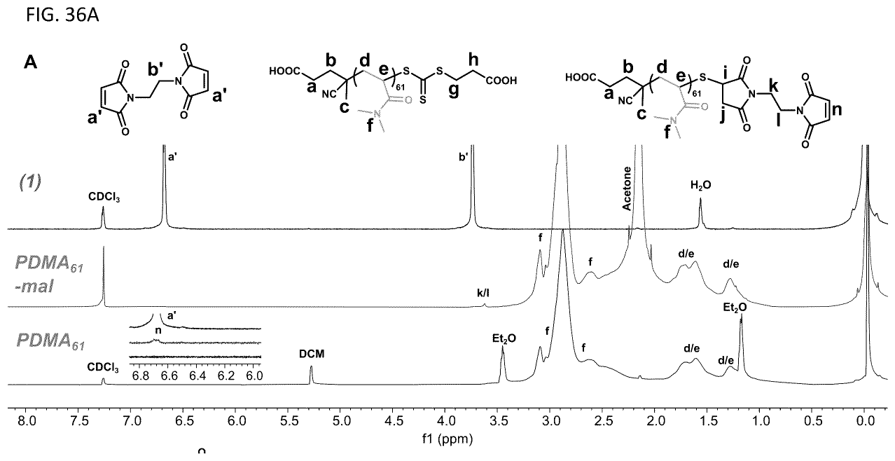

A total of five polymers were synthesized to interrogate the effects of architecture, degree of polymerization (n), and side chain length (m) on conjugate properties. All polymer number-averaged molecular weights (Mₙ) and polydispersity indices (Ð) were characterized by size exclusion chromatography (SEC) (FIG. 27C). Values for the PEG-maleimide polymer, which was previously conjugated to Gal3C[T243C] with a similar approach, are also included. The size exclusion chromatograms for each RAFT polymer are shown in FIG. 33. First, P(OEGMA⁵⁰⁰)₂₁ and PDMA₆₁ polymers were synthesized by targeting molecular weights that would result in SEC elution times comparable to that of the PEG polymer previously conjugated to Gal3C (5400 g/mol PEG-maleimide) (FIG. 34).³³ With this approach, hydrodynamic size was used as a control between the two polymers with different architectures and the previously studied PEG.

### Example 10: RAFT Polymerization of Polymer Series Prior to Functionalization

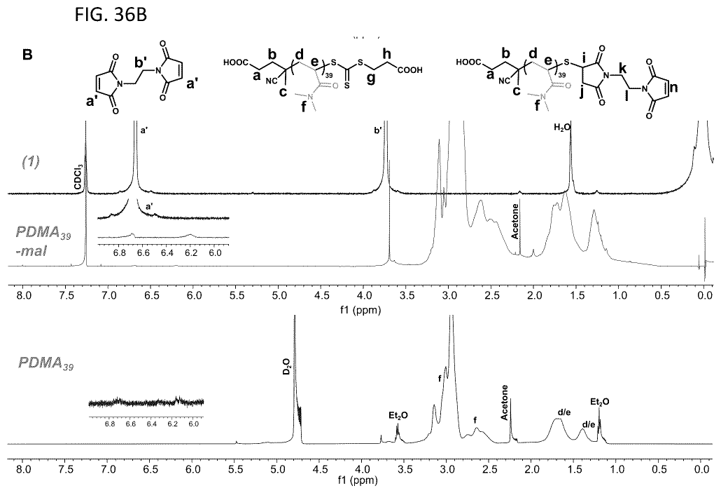

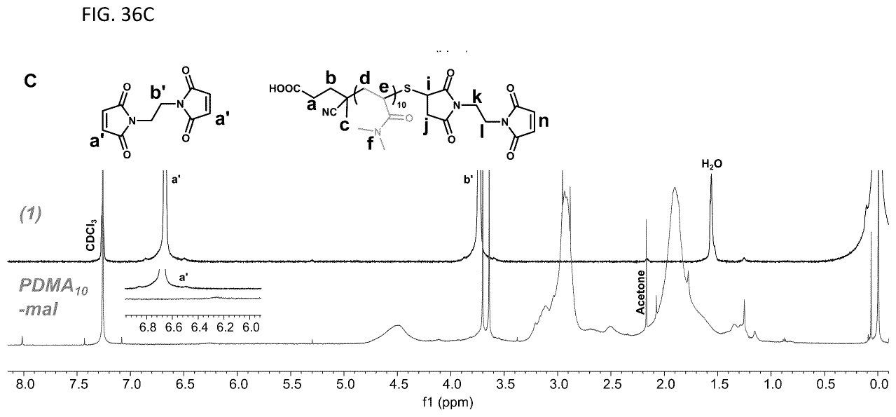

To study the effect of PDMA degree of polymerization (n) on Gal3C properties, intermediate-sized PDMA₁₀ and PDMA₃₉ were synthesized (FIG. 33A,B). To interrogate the effect of side chain length of comblike polymers on conjugate properties, a second OEGMA-based polymer with shorter side chains, P(OEGMA³⁰⁰)₂₀, was synthesized (FIG. 33E) at the approximate degree of polymerization (n) of P(OEGMA⁵⁰⁰)₂₁. Though the PDMA series featured lower dispersity as compared to the POEGMA series (FIG. 27C), SEC data indicated that all polymers were monodispersed before functionalization (FIG. 33). Assigned ¹H NMR spectra of each polymer confirmed their chemical identities (FIG. 35 and FIG. 36).

### Example 11: Polymer End-Group Modification

To rapidly functionalize the RAFT polymers with cysteine-reactive maleimide groups, a two-step end-group-functionalization scheme was employed (FIG. 32B,C). Aqueous desalting columns allowed rapid purification of small batches of polymer after each synthetic step. First, the trithiocarbonate end group was cleaved using hydrazine aminolysis to reveal a thiol group (FIG. 32B) following a modified procedure from Shen et al.³⁴ UV-vis spectra indicated that the trithiocarbonate peak at 315 nm disappeared after reacting with hydrazine, indicating near-quantitative conversion for each polymer (FIG. 37). The polymeric thiols were then functionalized with protein-reactive maleimide (FIG. 32C) by reacting with bismaleimide linker (1); linker structure was verified by ¹H NMR (FIGS. 38, 35 &36, blue traces) and purified via a desalting column. SEC data (FIG. 33, red traces) and ¹H NMR spectra (FIGS. 35 and 36, green traces) were used to characterize the final maleimide-functionalized polymers. After functionalization with 1, each POEGMA polymer ¹H NMR spectrum featured a doublet in the maleimide region at 6.72 ppm, which was integrated against the OEGMA monomer methyl peaks (3.35 ppm) and corresponded with 21% (POEGMA⁵⁰⁰₂₁-mal) and 12% (POEGMA³⁰⁰₂₀-mal) conversion to maleimide end group (FIG. 35A,B, green traces). The size exclusion chromatograms postfunctionalization (FIG. 33D,E) were monomodal, as expected for end-group functionalization with the bismaleimide linker (1). Additionally, the postfunctionalization SEC data of the POEGMA polymers showed a slight shift to higher elution times, indicating that most of the sample is the lower molecular weight polymeric thiol, as expected with only 12-21% functionalization of the bismaleimide.

As compared to the methacrylate POEGMA polymers, the acrylamide PDMA polymer series featured some polydispersity post-end-group modification. The two smallest PDMA polymers (PDMA₁₀-mal and PDMA₃₉-mal) featured multimodal SEC chromatograms postfunctionalization with 1 (FIG. 33A,B, respectively). These polymers were conjugated to Gal3C[T243C] without further purification because only the monomeric polymer species was expected to be protein-reactive. Postfunctionalization, the PDMA₆₁-mal chromatogram featured a monomodal peak with an elution time similar to that of polymer prefunctionalization (FIG. 331C). As with the chromatograms of POEGMA postfunctionalization, PDMA₆₁ exhibited a slight shift to a higher elution time due to the predominant product being the lower molecular weight polymeric thiol. The PDMA₆₁-mal ¹H NMR spectrum (FIG. 36A, green trace) featured a peak in the maleimide region at 6.62 ppm, and rough integration against the polymer N-methyl peaks (2.9 ppm) yielded a conversion of 8%. Due to the multimodal chromatograms observed for PDMA₁₀-mal and PDMA₃₉-mal, the ¹H NMR spectra were provided without full assignment (FIG. 36B,C, green traces). A ¹H NMR spectrum of PDMA₃₉-mal revealed a peak in the expected maleimide region at 6.67 ppm, indicating the presence of the proper end-group modification in one product. PDMA₁₀-mal was found to be the most polydispersed by SEC analysis, and a distinct maleimide peak was not resolved in its ¹H NMR spectrum, suggesting a lower percentage of monomeric polymer-maleimide compared to other products in the mixture. Despite this, the product mixture was still reacted with Gal3C[T243C] as large excess equivalents of the polymer were used, allowing for observed conjugate formation even though the desired end group was a minor product.

### Example 12: Grafting-To Conjugation with Gal3C[T243C]

The polymers were conjugated to Gal3C[T243C] with a grafting-to scheme (FIG. 27A). An excess of each aqueous polymer stock was used, with 10 equiv of reactive polymer-maleimide present to participate in a thiol-Michael addition with the solvent-exposed cysteine, C243, over the course of 4 h. Previously, it was determined that only C243 was functionalized with 5400 g/mol PEG-maleimide under similar grafting-to conditions with a shorter reaction time, and thus we anticipated site specificity would be preserved.³³

To mimic conditions used for conjugating RAFT-synthesized polymers herein, a control reaction using excess PEG-maleimide (10 equiv) was performed to assess for multiple additions of PEG after 4 h at room temperature (FIG. 39). A reaction time of 4.5 h showed mono-PEGylated Gal3C[T243C] as the primary product; only a minor amount of a higher molecular weight side product was detected with SEC (FIG. 39B) and SDS-PAGE at several crude reaction time points (FIG. 39C). MALDI-TOF revealed that this side product had a mass near the expected mass of a conjugate dimer (FIG. 39D). This dimer side product was hypothesized to be the result of contamination of manufacturer monomethoxy-PEG with PEG-diol.^{1,35} Overall, the vast majority of the product is Gal3C[T243C] with one addition of the polymer. This suggested that conjugation with non-PEG polymers would also result in single polymer attachments at site C243 in Gal3C[T243C].

### Example 13: Gal3C[T243C] Conjugate Purification and Assessment of Ligand Binding Function

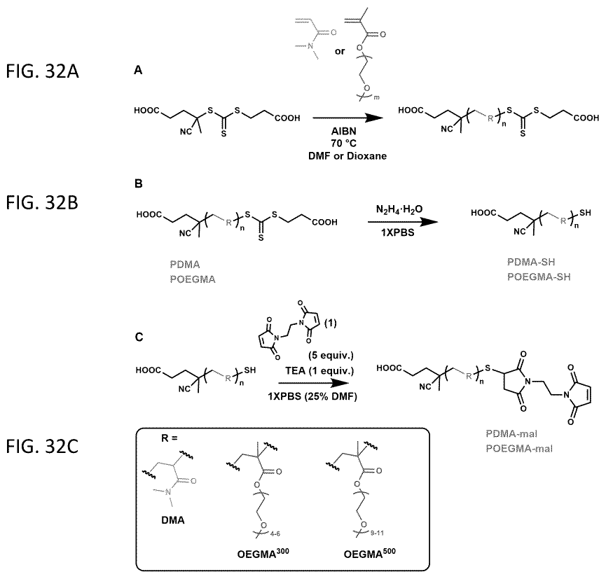

Gal3C[T243C] was first conjugated with the PEG analogues P(OEGMA⁵⁰⁰)₂₁ and PDMA₆₁, and conjugate protein function was assessed (FIG. 28). These two polymers were chosen to interrogate the effect of polymer architecture on conjugate properties and were designed to exhibit hydrodynamic size similar to that of 5400 g/mol PEG (FIG. 34). The conjugates were purified from unreacted polymers and protein with a final SEC purification step (FIG. 28B,C). Similar SEC elution times of each conjugate likely correlated with similar hydrodynamic radii. The dispersity of the conjugates, however, was not the same and corresponded with the dispersity of the attached polymer. The P(OEGMA⁵⁰⁰)₂₁ conjugate peak (FIG. 28B) was wider and therefore more disperse than the PDMA₆₁ conjugate peak (FIG. 28C). Fractions of the conjugate SEC peaks were selected for further characterization with SDS-PAGE (FIG. 28D,E), which revealed that each sample contained some unmodified Gal3C[T243C] protein (band near 17 kDa marker on ladder). Concentrated fractions were pooled and used to measure binding affinity to an endogenous Gal3C ligand, LacNac, via changes in inherent tryptophan fluorescence upon ligand binding (FIG. 27F) for W181 located adjacent to the ligand binding pocket, an established method for assessing Gal3C function³⁶ (FIG. 27F). Conjugation with either POEGMA or PDMA preserved protein function of Gal3C[T243C] with slightly increased binding affinities for LacNac over unmodified Gal3C[T243C] (FIG. 27F).

### Example 14: Assessment of Heterogeneity of Gal3C—Polymer Linker Chemistry

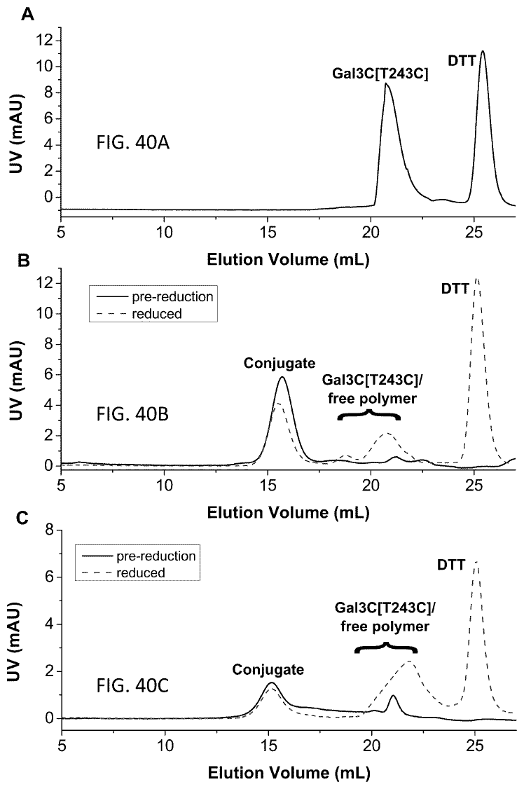

It was hypothesized that some of the conjugates were formed by disulfide bond coupling between C243 and polymer-thiols because the polymers featured subquantitative functionalization with the maleimide linker. Therefore, we wanted to assess if reducing conditions of SDS -PAGE enhanced the presence of the Gal3C[T243C] band by reducing disulfide bonds between polymer and protein. To interrogate this, individual fractions of each conjugate were selected, and SEC was performed pre- and postreduction with 10 mM DTT at 37° C. (FIG. 40). The SEC traces prereduction (FIG. 40B,C, black traces) revealed a small amount of Gal3C[T243C] in both conjugate samples. Postreduction (FIG. 40B,C, red dashed traces), the peak area of the conjugate decreased by 10-20% which indicated some disulfide bond linkages between the protein and polymer. Overall, this analysis suggested that conditions of the reducing SDS-PAGE gels presented herein may underestimate conjugate purity by 10-20%. Samples used for biophysical analyses were therefore prepared under nonreducing conditions. Minor heterogeneity in linker chemistry was not concerning based on previous work which suggests the linker has little effect on the galectin structure³³ or function.³⁷ HSQC spectra of Gal3C[T243C] conjugates did not show the presence of extensive peak doubling, as would be expected for heterogeneous samples containing a significant fraction of unconjugated protein (see below).

### Example 15: Mapping Gal3C—Polymer Interactions via NMR Spectroscopy in Aqueous Solutions

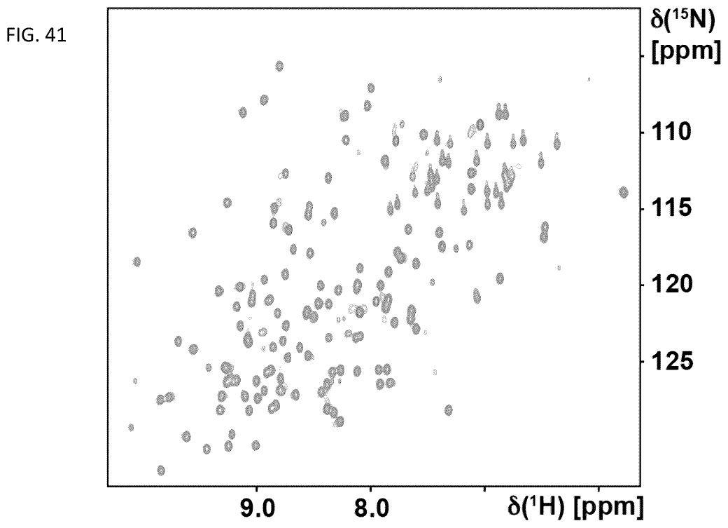

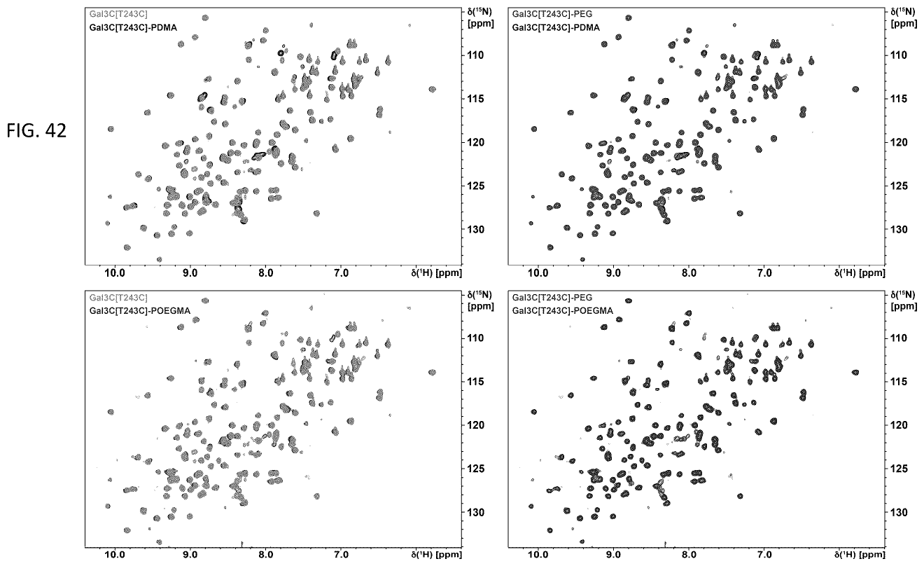

To provide a residue-specific assessment of the impact of polymer conjugation on the Gal3C backbone structure and conformational dynamics at an atomic scale, we prepared uniformly ¹⁵N-labeled Gal3C[T243C] conjugated to PDMA₆₁ or P(OEGMA⁵⁰⁰)₂₁ and recorded two-dimensional heteronuclear single-quantum correlation (HSQC) spectra. The HSQC spectra were recorded at 30° C. with both polymer conjugates and were well-dispersed and consistent with properly folded proteins (FIG. 41 and FIG. 42). Spectral resolution of both the unconjugated and conjugated proteins was also excellent, facilitating a careful comparison of the unconjugated and conjugated protein samples.

Because the chemical shifts of most signals for both conjugates were similar to those previously assigned for Gal3C,³⁸ Ga13,³⁹ and Gal3C[T243C]-PEG,³³ we could transfer information about the assignments to spectra of the present conjugates with PDMA₆₁ and P(OEGMA⁵⁰⁰)₂₁. This allowed us to quantitatively compare the impact of polymer conjugation on the protein backbone structure and dynamics by reporting chemical shift perturbations and changes in signal line widths, respectively, upon polymer conjugation. This also facilitated a comparison of the impact of Gal3C conjugation with PDMA₆₁ or P(OEGMA⁵⁰⁰)₂₁ to earlier results of Gal3C conjugated with PEG to reveal possible relationships between polymer architecture and response of the protein to conjugation with each polymer.³³

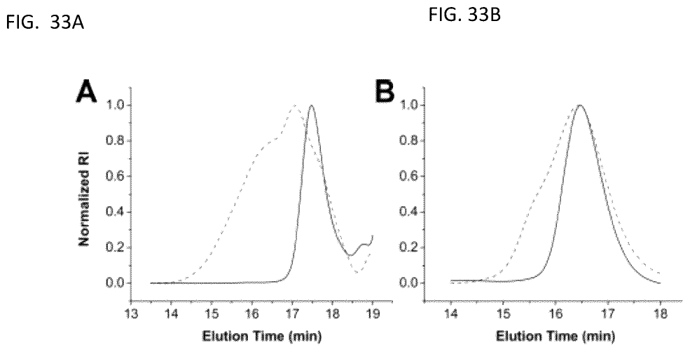

Comparison of HSQC spectra of unmodified [U-¹⁵N]-Gal3C[T243C] and [U-¹⁵N]-Gal3C[T243C] conjugated to either PDMA₆₁ or P(OEGMA⁵⁰⁰)₂₁ revealed distinct impacts of each polymer on the protein, which are dependent on the polymer chemical scaffold. The conjugation of [U-¹⁵N]-Gal3C[T243C] with the linear polymer PDMA₆₁ (FIG. 29B) resulted in patterns of chemical shift perturbations and line broadening that were highly similar to those observed for Gal3C[T243C] conjugated with PEG (FIG. 29A,D). The impact of conjugation of Gal3C with PDMA₆₁ was further visualized by mapping residues showing either significant chemical shift perturbations (>10 Hz) or line broadening onto a structure of Gal3C (PDB ID 4R9A)⁴⁰ (FIG. 29B, E). The largest impact was observed for residues local to the site of chemical conjugation, similar to Gal3C conjugates prepared with PEG,³³ and similar patterns of effects on the Gal3C backbone were observed for PEG and PDMA₆₁ (FIG. 29). Though monomers for PDMA₆₁ and PEG have different chemical structures (FIG. 27), both polymers have linear architecture, indicating that similar polymer architectures result in similar patterns of protein-polymer interactions (FIG. 29).

In contrast to the Gal3C[T243C] conjugate with PDMA₆₁, we observed more significant line broadening for a wider range of residues in NMR data of the conjugate with P(OEGMA⁵⁰⁰)₂₁ (FIG. 29C, F and FIG. 42). Broadening of signals in HSQC spectra of globular proteins has been attributed to fluctuations in the backbone conformation on the time scale of milliseconds to microseconds.⁴¹ Because we could not precisely delineate all potential contributions to line broadening, we focused on analyzing the patterns of protein-polymer interactions. For both conjugates with PDMA₆₁ and P(OEGMA⁵⁰⁰)₂₁, we observed significant line broadening and chemical shift differences for residues proximate to the site of conjugation. However, for the conjugate with P(OEGMA⁵⁰⁰)₂₁, more significant line broadening was observed among the same set of residues, especially for residues at positions 135-140 and positions 192-200, which primarily comprise loops connecting two adjacent β-strands (FIG. 29C,F). Interestingly, while conjugates prepared with PDMA and POEGMA shared similar sites of perturbations local to the conjugation site, Gal3C-POEGMA showed more extensive changes at residue positions farther from C243 (FIG. 29C,F). The polymer backbones for PDMA or PEG are linear and typically adopt a more random-coil-like conformation in solution. Conversely, POEGMA, with its numerous branches or side chains, adopts an extended, comblike conformation in solution. POEGMA can transition between extended or collapsed conformations depending on factors such as the length and chemical structure of its side chains, salt concentrations, or temperature.²¹ The NMR data for Gal3C conjugated to P(OEGMA⁵⁰⁰)₂₁ suggest a more extended conformation of the polymer in the conjugate, as changes in the HSQC NMR data were spread over a wider region of the protein surface (FIG. 29), as opposed to perturbations that would be more confined locally to the site of chemical conjugation for a more compact polymer backbone. For both conjugates, line broadening was observed to be specific to individual residues, not uniformly observed for the entire protein, indicating that local changes in protein dynamics caused by conjugation were likely responsible, rather than significant changes in the diffusion of the conjugated proteins.

### Example 16: Conjugation with PDMA and POEGMA Alters Gal3C Stability in a Polymer Length or Branch Length Dependent Manner

To interrogate the role of polymer length and chemical structure on the thermal stability of conjugated Gal3C[T243C], we recorded circular dichroism (CD) spectroscopy thermal melting assays with Gal3C[T243C] conjugated to each of the five polymers shown in FIG. 27C, including PDMA₆₁ and P(OEGMA⁵⁰⁰)₂₁. To determine the effect of polymer conjugation on the unfolding pathway of Gal3C[T243C], discrete fractions of Gal3C[T243C]-polymer conjugates from SEC experiments (Table S1) were collected and subjected to continuous ramp thermal melting monitored by CD spectroscopy. Melting profiles were determined from full CD versus wavelength spectra recorded with an applied linear temperature ramp from 30 to 90° C. for each conjugate (FIG. 43). Previously it was shown that conjugation of Gal3C[T243C] with PEG altered the unfolding pathway of Gal3C[T243C], resulting in a significantly higher thermal melting temperature (Tₘ) via the formation of a polymer-dependent unfolding intermediate.³³ It was thus of interest to investigate which factors were critical for improved protein stability, i.e., to what extent polymer architecture, degree of polymerization (n), or branch length (m) influenced the protein thermal unfolding profile.

TABLE S1
Exact fraction volumes of Gal30[T243C]
conjugates purified with SEC

| Conjugate | Fraction | Volume Range (mL) |
|---|---|---|
| Gal3C[T243C]-P(OEGMA⁵⁰⁰)₂₁ | A* | 14.06-14.39* |
| | B* | 14.39-14.72* |
| | C* | 15.05-15.38* |
| | D* | 15.71-16.04* |
| Gal3C[T243C]-PDMA₆₁ | 1* | 14.86-15.19* |
| | 2* | 15.19-15.52* |
| | 3* | 15.52-15.85* |
| | 4 | 15.85-16.18 |
| Gal3C[T2430]-P(OEGMA³⁰⁰)₂₀ | 1 | 15.89-16.22 |
| | 2* | 16.22-16.55* |
| | 3* | 16.55-16.88* |
| | 4* | 16.88-17.21* |
| | 5 | 17.21-17.54 |
| | 6 | 18.20-18.53 |
| Gal3C[T243C]-PDMA₁₀ | a | 16.55-16.88 |
| | b* | 16.88-17.21* |
| | c* | 17.21-17.54* |
| | d* | 17.54-17.87* |
| | e | 18.20-18.53 |
| Gal3C[T243C]-PDMA₃₉ | A | 15.05-15.38 |
| | B | 15.38-15.71 |
| | C* | 15.71-16.04* |
| | D* | 16.04-16.37* |
| | E* | 16.37-16.70* |

*Denotes samples used for CD thermal melting analysis. All fractions were 330 μL

To delineate the role of polymer length on thermal unfolding of Gal3C conjugated to linear polymers, Gal3C was conjugated to PDMA of three different degrees of polymerization (n=10, 39, and 61). Conjugated protein samples were evaluated via analytical SEC, SDS-PAGE, and CD-monitored thermal melting assays. Analytical SEC showed the presence of a peak for conjugated Gal3C that shifted left with increasing molecular weight, as expected (FIG. 30A-D). For n values of 39 and 61, the peak for modified Gal3C could be baseline resolved from unmodified Gal3C. For n=10, the modified and unmodified proteins were partially overlapped (FIG. 30B). Discrete fractions of each conjugate were characterized by SDS-PAGE (FIG. 30E-G) and thermal melting (FIG. 30H-J and Table S1) and compared with unmodified Gal3C[T243C].

FIG. 30H shows results of thermal melting of the PDMA₁₀ conjugate fractions b-d (FIG. 30B,E), which were selected to be enriched with conjugate rather than unmodified protein. Gal3C[T243C]PDMA₁₀ did not have observable intermediate formation, while Gal3C[T243C]-PDMA₃₉ (FIG. 301) and Gal3C[T243C]-PDMA₆₁ (FIG. 30J) did. Additionally, the two higher molecular weight PDMA conjugates had similar thermal stabilities and unfolding profiles. The thermal unfolding data indicated the oligomer PDMA₁₀ was not a large enough polymer to produce an intermediate, while PDMA₃₉ and PDMA₆₁ were long enough to significantly alter the Gal3C[T243C] thermal unfolding pathway. These results are reminiscent of earlier studies showing increased protein activity at higher temperatures with increased polymer chain lengths.^{23,29} From the thermal unfolding data, we concluded that a threshold polymer length for the linear PDMA was needed both for significant increased thermal stability and formation of a thermal unfolding intermediate.

Next, we used two comb-shaped POEGMA polymers to interrogate the effect of monomer branch length (m) on Gal3C thermal unfolding. We hypothesized that, for conjugates made with POEGMA, the polymer-polymer interactions of the methacrylate backbone and PEG oligo side chains may dominate over protein-polymer interactions, preventing the formation of thermal unfolding intermediates, especially for more hydrophobic, shorter branch length OEGMA³⁰⁰. Additionally, both POEGMA polymers exhibited a much lower n compared with intermediate forming polymers PDMA₆₁ and PEG. To directly test this, conjugates of Gal3C[T243C] were prepared with P(OEGMA⁵⁰⁰)₂₁ and P(OEGMA³⁰⁰)₂₀. Degree of polymerization (n) was controlled to assess the role of branch length (m) in protein unfolding.

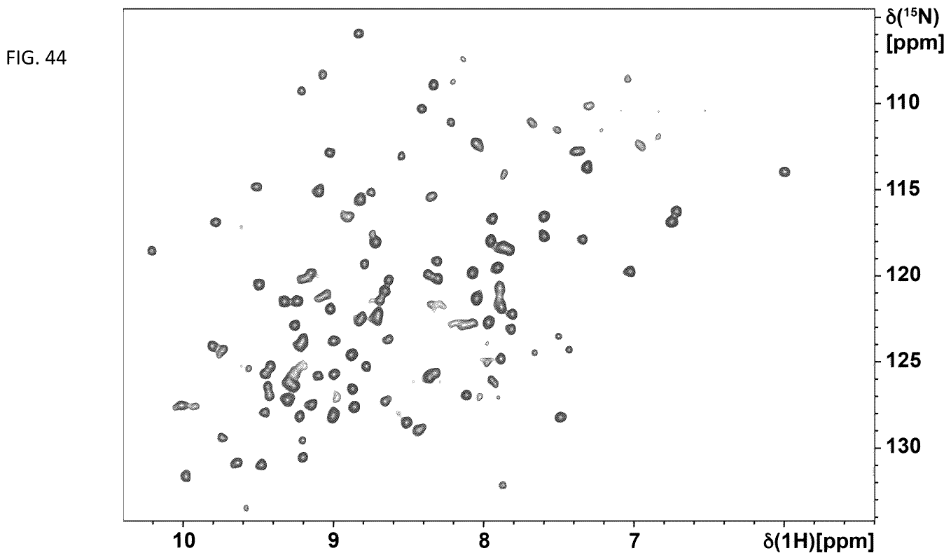

SEC separations of Gal3C[T243C]-P(OEGMA³⁰⁰)₂₀ and Gal3C[T243C]-P(OEGMA⁵⁰⁰)₂₁ were mostly resolved from unconjugated protein in SEC separations (FIG. 31B,C). Defined fractions of conjugated protein of varying molecular weight sizes were selected for SDS-PAGE analysis (FIG. 31D,E) and thermal melting (FIG. 31F,G). SDS-PAGE and SEC of the two conjugates showed consistent conjugate sizes as compared to unmodified Gal3C[T243C], which was observed as a contaminant in SDS-PAGE. The CD thermal melting assay revealed that all selected Gal3C[T243C]-P(OEGMA³⁰⁰)₂₀ fractions did not form defined intermediate states, but had a slightly higher overall melting temperature as compared to unmodified Gal3C[T243C] (FIG. 31F). Interestingly, thermal melting of Gal3C[T243C]-P(OEGMA⁵⁰⁰)₂₁ revealed a striking increase in Tₘ for conjugates of higher molecular weight fractions (FIG. 31C, fractions A and B). The same higher molecular weight fractions formed defined unfolding intermediate states, indicated by plateau regions from 65 to 80° C. (FIG. 31G). CD spectra of the intermediate state show that it is globally folded with mostly β sheet secondary structure (FIG. 43). The global fold of the intermediate is likely similar to that of the conjugated protein at lower temperature, but a shift in the λₘᵢₙ value from 222 to 218 nm over the intermediate formation temperature range indicates it is not identical. HSQC spectra of Gal3C[T243C]-P(OEGMA⁵⁰⁰)₂₁ measured at 55° C., the highest temperature we could safely operate the NMR probe, confirm the conjugated protein was folded (FIG. 44). The transition from a two-state to a three-state melting curve was previously observed for Gal3C[T243C] conjugated to PEG_{4.5k} and attributed to sustained protein-polymer interactions at increasing temperatures.³³

Lower molecular weight fractions of Gal3C[T243C]-P(OEGMA⁵⁰⁰)₂₁ (C and D) exhibited a qualitatively higher Tₘ than the unmodified protein but lacked the plateauing indicative of a defined intermediate (FIG. 31G, black circles and red squares). Comparison of thermal unfolding results from the two POEGMA-conjugated protein samples suggested the hydrophobic nature of P(OEGMA³⁰⁰)₂₀ (short branch length, m=4-6) may have reduced protein-polymer interactions required to redirect the protein thermal unfolding pathway. For the less hydrophobic and higher molecular weight P(OEGMA⁵⁰⁰)₂₁, a greater number of protein-polymer interactions may lead to the formation of a clear thermal unfolding intermediate state.

Comparing observations of thermal unfolding of Gal3C[T243C] conjugated to the linear PDMA and to the nonlinear POEGMA polymers enabled us to investigate potential correlations between polymer architecture and the thermal unfolding behavior of conjugated proteins. For both PDMA and POEGMA, a minimal polymer length was needed to form the thermal unfolding intermediate state. Interestingly, the intermediate states observed for both longer chain PDMA and POEGMA conjugates formed over similar temperature ranges. Intermediate states for both the PDMA and POEGMA conjugated protein and exhibited similar spectral signatures in the full CD versus wavelength melting spectra, as observed by the λ minimum shifting from 222 to 218 nm for the unfolding intermediate state (FIG. 43C,F). This suggested both polymers lead to similar intermediate global structures which are distinct from the native global structure. Because polymers can form amphiphilic interactions with the protein surface,^{33,42} it is conceivable that any neutral polymer above a certain threshold length may be able to form a sufficient amount of interactions with the protein to redirect the unfolding pathway, which may explain similar intermediate formation pathways shared among Gal3C[T243C] conjugated to P(OEGMA⁵⁰⁰)₂₁, PDMA₆₁, or PEG.

### REFERENCES

All references listed below and throughout the specification are hereby incorporated by reference in their entirety.
1. Anselmo, A. C., Gokarn, Y. & Mitragotri, S. Non-invasive delivery strategies for biologics. Nature Reviews Drug Discovery 18, 19-40 (2019).
2. Pham, J. V. et al. A Review of the Microbial Production of Bioactive Natural Products and Biologics. Front. Microbiol. 10, 1404 (2019).
3. Harris, J. M. & Chess, R. B. Effect of pegylation on pharmaceuticals. Nature Reviews Drug Discovery 2, 214-221 (2003).
4. Krall, N., Cruz, F. P.d., Boutureira, O. & Bernardes, G. J. L. Site-selective protein-modification chemistry for basic biology and drug development. Nat. Chem. 8, 103-113 (2016).
5. Shaunak, S. et al. Site-specific PEGylation of native disulfide bonds in therapeutic proteins. Nat. Chem. Biol. 2, 312-313 (2006).
6. Zhang, B. et al. Site-specific PEGylation of interleukin-2 enhances immunosuppression via the sustained activation of regulatory T cells. Nature Biomedical Engineering 5, 1288-1305 (2021).
7. Belén, L. H. et al. From Synthesis to Characterization of Site-Selective PEGylated Proteins. Front. Pharmacol. 10, 1450 (2019).
8. Grace, M. J. et al. Site of Pegylation and Polyethylene Glycol Molecule Size Attenuate Interferon-α Antiviral and Antiproliferative Activities through the JAK/STAT Signaling Pathway*. J. Biol. Chem. 280, 6327-6336 (2005).
9. Grigoletto, A., Mero, A., Zanusso, I., Schiavon, O. & Pasut, G. Chemical and Enzymatic Site Specific PEGylation of hGH: The Stability and in vivo Activity of PEG-N-Terminal-hGH and PEG-G1n141-hGH Conjugates. Macromol. Biosci. 16, 50-56 (2016).
10. Natalello, A. et al. Biophysical Characterization of Met-G-CSF: Effects of Different Site-Specific Mono-Pegylations on Protein Stability and Aggregation. PLoS One 7, e42511 (2012).
11. Rajan, R. S. et al. Modulation of protein aggregation by polyethylene glycol conjugation: GCSF as a case study. Protein Sci. 15, 1063-1075 (2006).
12. Zaghmi, A. et al. Mechanisms of activity loss for a multi-PEGylated protein by experiment and simulation. Materials Today Chemistry 12, 121-131 (2019).
13. Baker, S. L. et al. Intramolecular Interactions of Conjugated Polymers Mimic Molecular Chaperones to Stabilize Protein-Polymer Conjugates. Biomacromolecules 19, 3798-3813 (2018).
14. Chang, P. K., Prestidge, C. A., Barnes, T. J. & Bremmell, K. E. Impact of PEGylation and non-ionic surfactants on the physical stability of the therapeutic protein filgrastim (G-CSF). RSC Advances 6, 78970-78978 (2016).
15. Hauptstein, N. et al. Molecular Insights into Site-Specific Interferon-α2a Bioconjugates Originated from PEG, LPG, and PEtOx. Biomacromolecules 22, 4521-4534 (2021).
16. Pai, S. S. et al. The Conformation of the Poly(ethylene glycol) Chain in Mono-PEGylated Lysozyme and Mono-PEGylated Human Growth Hormone. Bioconjugate Chemistry 22, 2317-2323 (2011).
17. Cœur, C. m. L. et al. Conformation of the Poly(ethylene Glycol) Chains in DiPEGylated Hemoglobin Specifically Probed by SANS: Correlation with PEG Length and in Vivo Efficiency. Langmuir 31, 8402-8410 (2015).
18. He, L. et al. Analysis of MonoPEGylated Human Galectin-2 by Small-Angle X-ray and Neutron Scattering: Concentration Dependence of PEG Conformation in the Conjugate. Biomacromolecules 11, 3504-3510 (2010).
19. Shu, J. Y., Lund, R. & Xu, T. Solution Structural Characterization of Coiled-Coil Peptide-Polymer Side-Conjugates. Biomacromolecules 13, 1945-1955 (2012).
20. Ferebee, R. et al. Light Scattering Analysis of Mono- and Multi-PEGylated Bovine Serum Albumin in Solution: Role of Composition on Structure and Interactions. The Journal of Physical Chemistry B 120, 4591-4599 (2016).
21. Dhalluin, C. et al. Structural and Biophysical Characterization of the 40 kDa PEG-Interferon-α2 a and Its Individual Positional Isomers. Bioconjugate Chemistry 16, 504-517 (2005).
22. Munasinghe, A., Mathavan, A., Mathavan, A., Lin, P. & Colina, C. M. PEGylation within a confined hydrophobic cavity of a protein. Phys. Chem. Chem. Phys. 21, 25584-25596 (2019).
23. Yang, C., Lu, D. & Liu, Z. How PEGylation Enhances the Stability and Potency of Insulin: A Molecular Dynamics Simulation. Biochemistry 50, 2585-2593 (2011).
24. Munasinghe, A., Mathavan, A., Mathavan, A., Lin, P. & Colina, C. M. Molecular Insight into the Protein-Polymer Interactions in N-Terminal PEGylated Bovine Serum Albumin. The Journal of Physical Chemistry B 123, 5196-5205 (2019).
25. Farhadi, S. A. et al. Locally anchoring enzymes to tissues via extracellular glycan recognition. Nature Communications 9, 4943 (2018).
26. Newlaczyl, A. U. & Yu, L.-G. Galectin-3—A jack-of-all-trades in cancer. Cancer Lett. 313, 123-128 (2011).
27. Yu, L.-G. et al. Galectin-3 Interaction with Thomsen-Friedenreich Disaccharide on Cancer-associated MUC1 Causes Increased Cancer Cell Endothelial Adhesion. J. Biol. Chem. 282, 773-781 (2007).
28. Nakahara, S., Oka, N. & Raz, A. On the role of galectin-3 in cancer apoptosis. Apoptosis 10, 267-275 (2005).
29. Takenaka, Y., Fukumori, T. & Raz, A. Galectin-3 and metastasis. Glycoconj. J. 19, 543-549 (2002).
30. Reesink, H. L. et al. Galectin-3 Binds to Lubricin and Reinforces the Lubricating Boundary Layer of Articular Cartilage. Sci. Rep. 6, 25463 (2016).
31. Mattos, R. M. d. et al. Galectin-3 plays an important role in endometriosis development and is a target to endometriosis treatment. Molecular and Cellular Endocrinology 486, 1-10 (2019).
32. John, C. M., Leffler, H , Kahl-Knutsson, B., Svensson, I. & Jarvis, G. A. Truncated Galectin-3 Inhibits Tumor Growth and Metastasis in Orthotopic Nude Mouse Model of Human Breast Cancer. 10 (2003).
33. Mirandola, L. et al. Galectin-3C Inhibits Tumor Growth and Increases the Anticancer Activity of Bortezomib in a Murine Model of Human Multiple Myeloma. PLoS One 6, e21811 (2011).
34. García-Arellano, H., Valderrama, B., Saab-Rincón, G. & Vazquez-Duhalt, R. High Temperature Biocatalysis by Chemically Modified Cytochrome c. Bioconjugate Chemistry 13, 1336-1344 (2002).
35. Wang, Y. & Wu, C. Site-Specific Conjugation of Polymers to Proteins. Biomacromolecules 19, 1804-1825 (2018).
36. Ravasco, J. M. J. M., Faustino, H., Trindade, A. & Gois, P. M. P. Bioconjugation with Maleimides: A Useful Tool for Chemical Biology. Chemistry—A European Journal 25, 43-59 (2019).
37. Renault, K. v., Fredy, J. W., Renard, P.-Y. & Sabot, C. Covalent Modification of Biomolecules through Maleimide-Based Labeling Strategies. Bioconjugate Chemistry 29, 2497-2513 (2018).
38. Restuccia, A., Tian, Y. F., Collier, J. H. & Hudalla, G. A. Self-Assembled Glycopeptide Nanofibers as Modulators of Galectin-1 Bioactivity. Cell. Mol. Bioeng. 8, 471-487 (2015).
39. Seetharaman, J. et al. X-ray Crystal Structure of the Human Galectin-3 Carbohydrate Recognition Domain at 2.1-Å Resolution*. J. Biol. Chem. 273, 13047-13052 (1998).
40. Umemoto, K. & Leffler, H. Assignment of 1H, 15N and 13C resonances of the carbohydrate recognition domain of human galectin-3. J. Biomol. NMR 20, 91-92 (2001).
41. Ippel, H. et al. Intra- and intermolecular interactions of human galectin-3: assessment by full-assignment-based NMR. Glycobiology 26, 888-903 (2016).
42. Aramini, J. M. et al. Structural basis of O6-alkylguanine recognition by a bacterial alkyltransferase-like DNA repair protein. J. Biol. Chem. 285, 13736-41 (2010).
43. Rossi, P. et al. A microscale protein NMR sample screening pipeline. J. Biomol. NMR 46, 11-22 (2010).
44. Jain, A. & Ashbaugh, H. S. Helix Stabilization of Poly(ethylene glycol)-Peptide Conjugates. Biomacromolecules 12, 2729-2734 (2011).
45. Hamed, E., Xu, T. & Keten, S. Poly(ethylene glycol) Conjugation Stabilizes the Secondary Structure of cc-Helices by Reducing Peptide Solvent Accessible Surface Area. Biomacromolecules 14, 4053-4060 (2013).
46. Morgenstern, J., Baumann, P., Brunner, C. & Hubbuch, J. Effect of PEG molecular weight and PEGylation degree on the physical stability of PEGylated lysozyme. Int. J. Pharm. 519, 408-417 (2017).
47. Plesner, B., Fee, C. J., Westh, P. & Nielsen, A. D. Effects of PEG size on structure, function and stability of PEGylated BSA. Eur. J. Pharm. Biopharm. 79, 399-405 (2011).
48. Lawrence, P. B. et al. Criteria for Selecting PEGylation Sites on Proteins for Higher Thermodynamic and Proteolytic Stability. J. Am. Chem. Soc. 136, 17547-17560 (2014).
49. Draper, S. R. E. et al. Polyethylene Glycol Based Changes to β-Sheet Protein Conformational and Proteolytic Stability Depend on Conjugation Strategy and Location. Bioconjugate Chemistry 28, 2507-2513 (2017).
50. Lawrence, P. B. et al. Conjugation Strategy Strongly Impacts the Conformational Stability of a PEG-Protein Conjugate. ACS Chem. Biol. 11, 1805-1809 (2016).
51. Cattani, G., Vogeley, L. & Crowley, P. B. Structure of a PEGylated protein reveals a highly porous double-helical assembly. Nat. Chem. 7, 823-828 (2015).
52. Lin, Y. et al. J. Biol. Chem. 2017, 29217845-17856
53. Seetharaman, J.; et al. J. Biol.Chem. 1998,273(21), 13047-13052.
54. John; Leffler; Kahl-Knutsson; Svensson; Jarvis; “Truncated Galectin-3 Inhibits Tumor Growth and Metastasis in Orthotopic Nude Mouse Model of Human Breast Cancer” Clinical Cancer Research (2003), 9, 2374-2383.
55. Mirandola; Yu; Chui; Jenkins; Cobos; John; Chiriva-Internati; “Galectin-3C Inhibits Tumor Growth and Increases the Anticancer Activity of Bortezomib in a Murine Model of Human Multiple Myeloma” PLOS one (2011), 6, e21811.
56. Barnes, B. E.; Jenkins, T. A.; Stein, L. M.; Mathers, R. T.; Wicaksana, M.; Pasquinelli, M. A.; Savin, D. A., Synthesis and Characterization of a Leucine-Based Block Co-Polypeptide: The Effect of the Leucine Zipper on Self-Assembly. Biomacromolecules 2020, 21 (6), 2463-2472.
57. Restuccia, A.; Tian, Y. F.; Collier, J. H.; Hudalla, G. A., Self-Assembled Glycopeptide Nanofibers as Modulators of Galectin-1 Bioactivity. Cell. Mol. Bioeng. 2015, 8 (3), 471-487.
58. Umemoto, K.; Leffler, H., Assignment of 1H, 15N and 13C resonances of the carbohydrate recognition domain of human galectin-3. J. Biomol. NMR 2001, 20 (1), 91-92.
59. Ippel, H.; Miller, M. C.; Vértesy, S.; Zheng, Y.; Canada, F. J.; Suylen, D.; Umemoto, K.; Romanò, C.; Hackeng, T.; Tai, G.; Leffler, H.; Kopitz, J.; André, S.; Kübler, D.; Jiménez-Barbero, J.; Oscarson, S.; Gabius, H.-J.; Mayo, K. H., Intra- and intermolecular interactions of human galectin-3: assessment by full-assignment-based NMR. Glycobiology 2016, 26 (8), 888-903.
60. Czaplicki, J.; Comelissen, G.; Halberg, F., GOSA, a simulated annealing-based program for global optimization of nonlinear problems, also reveals transyears. J. Appl. Biomed. 2006, 4 (2), 87-94.
61. Lipari, G.; Szabo, A., Model-free approach to the interpretation of nuclear magnetic resonance relaxation in macromolecules. 2. Analysis of experimental results. J. Am. Chem. Soc. 1982, 104 (17), 4559-4570.
62. Aramini, J. M.; Tubbs, J. L.; Kanugula, S.; Rossi, P.; Ertekin, A.; Maglaqui, M.; Hamilton, K.; Ciccosanti, C. T.; Jiang, M.; Xiao, R.; Soong, T.-T.; Rost, B.; Acton, T. B.; Everett, J. K.; Pegg, A. E.; Tainer, J. A.; Montelione, G. T., Structural basis of O6-alkylguanine recognition by a bacterial alkyltransferase-like DNA repair protein. J. Biol. Chem. 2010, 285 (18), 13736-41.
63. Rossi, P.; Swapna, G. V. T.; Huang, Y. J.; Aramini, J. M.; Anklin, C.; Conover, K.; Hamilton, K.; Xiao, R.; Acton, T. B.; Ertekin, A.; Everett, J. K.; Montelione, G. T., A microscale protein NMR sample screening pipeline. J. Biomol. NMR 2010, 46 (1), 11-22.
64. Su, J.; Zhang, T.; Wang, P.; Liu, F.; Tai, G.; Zhou, Y., The water network in galectin-3 ligand binding site guides inhibitor design. Acta Biochimica et Biophysica Sinica 2015, 47 (3), 192-198.
65. Dimitrov et al., Methods Mol. Biol., 2012. Munasinghe et al., J. Phys. Chem. B, 2019. Dong et al., Int. J. Mol. Med., 2017.

### References for Example 8

(1) Sava, M. Preparation and characterization of bismaleimide monomers with various structures. Designed Monomers and Polymers 2013, 16 (1), 14-24. DOI: 10.1080/15685551.2012.705485.
(2) Cava, M. P.; Deana, A. A.; Muth, K.; Mitchell, M. J. N-PHENYLMALEIMIDE. Organic Syntheses 1973, 5, 944.
(3) Shen, W.; Qiu, Q.; Wang, Y.; Miao, M.; Li, B.; Zhang, T.; Cao, A.; An, Z. Hydrazine as a Nucleophile and Antioxidant for Fast Aminolysis of RAFT Polymers in Air. Macromolecular Rapid Communications 2010, 31 (16), 1444-1448, https://doi.org/10.1002/marc.201000154. DOI: https://doi.org/10.1002/marc.201000154 (acccessed 2022 Dec. 10).
(4) Pritzlaff, A.; Ferre, G.; Mulry, E.; Lin, L.; Gopal Pour, N.; Savin, D. A.; Harris, M. E.; Eddy, M. T. Atomic-Scale View of Protein-PEG Interactions that Redirect the Thermal Unfolding Pathway of PEGylated Human Galectin-3. Angewandte Chemie International Edition 2022, 61 (40), e202203784, https://doi.or /10.1002/anie.202203784. DOI: https://doi.org/10.1002/anie.202203784 (acccessed2022 Dec. 8).

### References for Examples 9-16

1. Harris J. M.; Chess R. B. Effect of pegylation on pharmaceuticals. Nat. Rev. Drug Discovery 2003, 2 (3), 214-221. 10.1038/nrd1033. [PubMed] [CrossRef] [Google Scholar]
2. Wright T. A.; Page R. C.; Konkolewicz D. Polymer conjugation of proteins as a synthetic post-translational modification to impact their stability and activity. Polym. Chem. 2019, 10 (4), 434-454. 10.1039/C8PY01399C. [PMC free article] [PubMed] [CrossRef] [Google Scholar]
3. Anselmo A. C.; Gokarn Y.; Mitragotri S. Non-invasive delivery strategies for biologics. Nat. Rev. Drug Disc. 2019, 18 (1), 19-40. 10.1038/nrd.2018.183. [PubMed] [CrossRef] [Google Scholar]
4. Treetharnmathurot B.; Ovartlarnporn C.; Wungsintaweekul J.; Duncan R.; Wiwattanapatapee R. Effect of PEG molecular weight and linking chemistry on the biological activity and thermal stability of PEGylated trypsin. Int. J. Pharm. 2008, 357 (1-2), 252-259. 10.1016/j.ijpharm.2008.01.016. [PubMed] [CrossRef] [Google Scholar]
5. Lee P.; Towslee J.; Maia J.; Pokorski J. PEGylation to Improve Protein Stability During Melt Processing. Macromol. Biosci. 2015, 15 (10), 1332-1337. 10.1002/mabi.201500143. [PMC free article] [PubMed] [CrossRef] [Google Scholar]
6. Krall N.; da Cruz F. P.; Boutureira O.; Bernardes G. J. L. Site-selective protein-modification chemistry for basic biology and drug development. Nat. Chem. 2016, 8 (2), 103-113. 10.1038/nchem.2393. [PubMed] [CrossRef] [Google Scholar]
7. Zhang B.; Sun J.; Wang Y.; Ji D.; Yuan Y.; Li S.; Sun Y.; Hou Y.; Li P.; Zhao L.; et al. Site-specific PEGylation of interleukin-2 enhances immunosuppression via the sustained activation of regulatory T cells. Nature Biomedical Engineering 2021, 5 (11), 1288-1305. 10.1038/s41551-021-00797-8. [PubMed] [CrossRef] [Google Scholar]
8. Tenchov R.; Bird R.; Curtze A. E.; Zhou Q. Lipid Nanoparticles—From Liposomes to mRNA Vaccine Delivery, a Landscape of Research Diversity and Advancement. ACS Nano 2021, 15 (11), 16982-17015. 10.1021/acsnano.1c04996. [PubMed] [CrossRef] [Google Scholar]
9. Ju Y.; Lee W. S.; Pilkington E. H.; Kelly H. G.; Li S.; Selva K. J.; Wragg K. M.; Subbarao K.; Nguyen T. H. O.; Rowntree L. C.; et al. Anti-PEG Antibodies Boosted in Humans by SARS-CoV-2 Lipid Nanoparticle mRNA Vaccine. ACS Nano 2022, 16 (8), 11769-11780. 10.1021/acsnano.2c04543. [PMC free article] [PubMed] [CrossRef] [Google Scholar]
10. Kozma G. T.; Shimizu T.; Ishida T.; Szebeni J. Anti-PEG antibodies: Properties, formation, testing and role in adverse immune reactions to PEGylated nano-biopharmaceuticals. Adv. Drug Delivery Rev. 2020, 154-155, 163-175. 10.1016/j.addr.2020.07.024. [PubMed] [CrossRef] [Google Scholar]
11. Chen E.; Chen B.-M.; Su Y.-C.; Chang Y.-C.; Cheng T.-L.; Barenholz Y.; Roffler S. R. Premature Drug Release from Polyethylene Glycol (PEG)-Coated Liposomal Doxorubicin via Formation of the Membrane Attack Complex. ACS Nano 2020, 14 (7), 7808-7822. 10.1021/acsnano.9b07218. [PubMed] [CrossRef] [Google Scholar]
12. Olson R. A.; Korpusik A. B.; Sumerlin B. S. Enlightening advances in polymer bioconjugate chemistry: light-based techniques for grafting to and from biomacromolecules. Chemical Science 2020, 11 (20), 5142-5156. 10.1039/DOSC01544J. [PMC free article] [PubMed] [CrossRef] [Google Scholar]
13. Russell A. J.; Baker S. L.; Colina C. M.; Figg C. A.; Kaar J. L.; Matyjaszewski K.; Simakova A.; Sumerlin B. S. Next generation protein-polymer conjugates. AIChE J. 2018, 64 (9), 3230-3245. 10.100 2/aic.16338. [CrossRef] [Google Scholar]
14. Perrier S. 50th Anniversary Perspective: RAFT Polymerization—A User Guide. Macromolecules 2017, 50 (19), 7433-7447. 10.1021/acs.macromo1.7b00767. [CrossRef] [Google Scholar]
15. Boyer C.; Bulmus V.; Davis T. P.; Ladmiral V.; Liu J.; Perrier S. Bioapplications of RAFT Polymerization. Chem. Rev. 2009, 109 (11), 5402-5436. 10.1021/cr9001403. [PubMed] [CrossRef] [Google Scholar]
16. Kierstead P. H.; Okochi H.; Venditto V. J.; Chuong T. C.; Kivimae S.; Fréchet J. M. J.; Szoka F. C. The effect of polymer backbone chemistry on the induction of the accelerated blood clearance in polymer modified liposomes. J. Controlled Release 2015, 213, 1-9. 10.1016/j.jconre1.2015.06.023. [PMC free article] [PubMed] [CrossRef] [Google Scholar]
17. Hoang Thi T. T.; Pilkington E. H.; Nguyen D. H.; Lee J. S.; Park K. D.; Truong N. P. The Importance of Poly(ethylene glycol) Alternatives for Overcoming PEG Immunogenicity in Drug Delivery and Bioconjugation. Polymers 2020, 12 (2), 298. 10.3390/polym12020298. [PMC free article] [PubMed] [CrossRef] [Google Scholar]
18. Joh D. Y.; Zimmers Z.; Avlani M.; Heggestad J. T.; Aydin H. B.; Ganson N.; Kumar S.; Fontes C. M.; Achar R. K.; Hershfield M. S.; et al. Architectural Modification of Conformal PEG-Bottlebrush Coatings Minimizes Anti-PEG Antigenicity While Preserving Stealth Properties. Adv. Healthcare Mater. 2019, 8 (8), 1801177 10.1002/adhm.201801177. [PMC free article] [PubMed] [CrossRef] [Google Scholar]
19. Wright T. A.; Rahman M. S.; Bennett C.; Johnson M. R.; Fischesser H.; Ram N.; Tyler A.; Page R. C.; Konkolewicz D. Hydrolytically Stable Maleimide-End-Functionalized Polymers for Site-Specific Protein Conjugation. Bioconjugate Chem. 2021, 32 (11), 2447-2456. 10.1021/acs.bioconjchem.1c00487. [PMC free article] [PubMed] [CrossRef] [Google Scholar]
20. Rahman M. S.; Brown J.; Murphy R.; Carnes S.; Carey B.; Averick S.; Konkolewicz D.; Page R. C. Polymer Modification of Lipases, Substrate Interactions, and Potential Inhibition. Biomacromolecules 2021, 22 (2), 309-318. 10.1021/acs.biomac.0c01159. [PubMed] [CrossRef] [Google Scholar]
21. Baker S. L.; Munasinghe A.; Kaupbayeva B.; Rebecca Kang N.; Certiat M.; Murata H.; Matyjaszewski K.; Lin P.; Colina C. M.; Russell A. J. Transforming protein-polymer conjugate purification by tuning protein solubility. Nat. Commun. 2019, 10 (1), 4718. 10.1038/s41467-019-12612-9. [PMC free article] [PubMed] [CrossRef] [Google Scholar]
22. Liu M.; Leroux J.-C.; Gauthier M. A. Conformation-function relationships for the comb-shaped polymer pOEGMA. Prog. Polym. Sci. 2015, 48, 111-121. 10.1016/j .progpolymsci.2015.03.001. [CrossRef] [Google Scholar]
23. Wright T. A.; Bennett C.; Johnson M. R.; Fischesser H.; Chandrarathne B. M.; Ram N.; Maloof E.; Tyler A.; Upshaw C. R.; Stewart J. M.; et al. Investigating the Impact of Polymer Length, Attachment Site, and Charge on Enzymatic Activity and Stability of Cellulase. Biomacromolecules 2022, 23 (10), 4097-4109. 10.1021/acs.biomac.2c00441. [PubMed] [CrossRef] [Google Scholar]
24. Piçarra S.; Relogio P.; Afonso C. A. M.; Martinho J. M. G.; Farinha J. P. S. Coil-Globule Transition of Poly(Dimethylacrylamide): Fluorescence and Light Scattering Study. Macromolecules 2003, 36 (21), 8119-8129. 10.1021/ma0345899. [CrossRef] [Google Scholar]
25. Nasseri R.; Tam K. C. Sticky Hydrogels from Hydrazide-Functionalized Poly(oligo(ethylene glycol) methacrylate) and Dialdehyde Cellulose Nanocrystals with Tunable Thermal and Strain-Hardening Characteristics. ACS Sustainable Chem. Eng. 2021, 9 (31), 10424-10430. 10.1021/acssuschemeng.1c03183. [CrossRef] [Google Scholar]
26. Roy D.; Brooks W. L. A.; Sumerlin B. S. New directions in thermoresponsive polymers. Chem. Soc. Rev. 2013, 42 (17), 7214-7243. 10.1039/c3cs35499g. [PubMed] [CrossRef] [Google Scholar]
27. Liu M.; Tirino P.; Radivojevic M.; Phillips D. J.; Gibson M. I.; Leroux J.-C.; Gauthier M. A. Molecular Sieving on the Surface of a Protein Provides Protection Without Loss of Activity. Adv. Funct. Mater. 2013, 23 (16), 2007-2015. 10.1002/adfm.201202227. [CrossRef] [Google Scholar]
28. Qi Y.; Chilkoti A. Protein-polymer conjugation—moving beyond PEGylation. Curr. Opin. Chem. Biol. 2015, 28, 181-193. 10.1016/j.cbpa.2015.08.009. [PMC free article] [PubMed] [CrossRef] [Google Scholar]
29. Cao L.; Shi X.; Cui Y.; Yang W.; Chen G.; Yuan L.; Chen H. Protein-polymer conjugates prepared via host-guest interactions: effects of the conjugation site, polymer type and molecular weight on protein activity. Polym. Chem. 2016, 7 (32), 5139-5146. 10.1039/C6PY00882H. [CrossRef] [Google Scholar]
30. Wright T. A.; Lucius Dougherty M.; Schmitz B.; Burridge K. M.; Makaroff K.; Stewart J. M.; Fischesser H. D.; Shepherd J. T.; Berberich J. A.; Konkolewicz D.; et al. Polymer Conjugation to Enhance Cellulase Activity and Preserve Thermal and Functional Stability. Bioconjugate Chem. 2017, 28 (10), 2638-2645. 10.1021/acs.bioconjchem.7b00518. [PubMed] [CrossRef] [Google Scholar]
31. Cummings C. S.; Campbell A. S.; Baker S. L.; Carmali S.; Murata H.; Russell A. J. Design of Stomach Acid-Stable and Mucin-Binding Enzyme Polymer Conjugates. Biomacromolecules 2017, 18 (2), 576-586. 10.1021/acs.biomac.6b01723. [PubMed] [CrossRef] [Google Scholar]
32. Lucius M.; Falatach R.; McGlone C.; Makaroff K.; Danielson A.; Williams C.; Nix J. C.; Konkolewicz D.; Page R. C.; Berberich J. A. Investigating the Impact of Polymer Functional Groups on the Stability and Activity of Lysozyme-Polymer Conjugates. Biomacromolecules 2016, 17 (3), 1123-1134. 10.1021/acs.biomac.5b01743. [PubMed] [CrossRef] [Google Scholar]
33. Pritzlaff A.; Ferré G.; Mulry E.; Lin L.; Gopal Pour N.; Savin D. A.; Harris M. E.; Eddy M. T. Atomic-Scale View of Protein-PEG Interactions that Redirect the Thermal Unfolding Pathway of PEGylated Human Galectin-3. Angew. Chem., Int. Ed. 2022, 61 (40), e202203784 10.1002/anie.202203784. [PMC free article] [PubMed] [CrossRef] [Google Scholar]
34. Shen W.; Qiu Q.; Wang Y.; Miao M.; Li B.; Zhang T.; Cao A.; An Z. Hydrazine as a Nucleophile and Antioxidant for Fast Aminolysis of RAFT Polymers in Air. Macromol. Rapid Commun. 2010, 31 (16), 1444-1448. 10.1002/marc.201000154. [PubMed] [CrossRef] [Google Scholar]
35. Veronese F. M.; Mero A. The Impact of PEGylation on Biological Therapies. BioDrugs 2008, 22 (5), 315-329. 10.2165/00063030-200822050-00004. [PubMed] [CrossRef] [Google Scholar]
36. Restuccia A.; Tian Y. F.; Collier J. H.; Hudalla G. A. Self-assembled glycopeptide nanofibers as modulators of galectin-1 bioactivity. Cellular and molecular bioengineering 2015, 8 (3), 471-487. 10.1007/s12195-015-0399-2. [PMC free article] [PubMed] [CrossRef] [Google Scholar]
37. Kane B. J.; Fettis M. M.; Farhadi S. A.; Liu R.; Hudalla G. A. Site-Specific Cross-Linking of Galectin-1 Homodimers via Poly(ethylene glycol) Bismaleimide. Cellular and Molecular Bioengineering 2021, 14, 523. 10.1007/s12195-021-00681-0. [PMC free article] [PubMed] [CrossRef] [Google Scholar]
38. Umemoto K.; Leffler H. Assignment of 1H, 15N and 13C resonances of the carbohydrate recognition domain of human galectin-3. J. Biomol NMR 2001, 20 (1), 91-92. 10.1023/A:1011269008175. [PubMed] [CrossRef] [Google Scholar]
39. Ippel H.; Miller M. C.; Vertesy S.; Zheng Y.; Canada F. J.; Suylen D.; Umemoto K.; Romanò C.; Hackeng T.; Tai G.; et al. Intra- and intermolecular interactions of human galectin-3: assessment by full-assignment-based NMR. Glycobiology 2016, 26 (8), 888-903. 10.1093/glycob/cww021. [PMC free article] [PubMed] [CrossRef] [Google Scholar]
40. Su J.; Zhang T.; Wang P.; Liu F.; Tai G.; Zhou Y. The water network in galectin-3 ligand binding site guides inhibitor design. Acta Biochim Biophys Sin (Shanghai) 2015, 47 (3), 192-198. 10.1093/abbs/gmu132. [PubMed] [CrossRef] [Google Scholar]
41. Waudby C. A.; Ramos A.; Cabrita L. D.; Christodoulou J. Two-Dimensional NMR Lineshape Analysis. Sci. Rep. 2016, 6 (1), 24826. 10.1038/srep24826. [PMC free article] [PubMed] [CrossRef] [Google Scholar]
42. Munasinghe A.; Mathavan A.; Mathavan A.; Lin P.; Colina C. M. Molecular Insight into the Protein—Polymer Interactions in N-Terminal PEGylated Bovine Serum Albumin J Phys. Chem. B 2019, 123 (25), 5196-5205. 10.1021/acs.jpcb.8b12268. [PubMed] [CrossRef] [Google Scholar]

What is claimed is:

1. An agent comprising galectin 3C that comprises at least one substituted cysteine residue substituted in place of a rationally-selected and/or solvent accessible residue, wherein the galectin 3C is conjugated to a polymer at the at least one substituted cysteine residue.

2. The agent of claim 1, wherein the polymer is PEG, PDMA, or POEGMA.

3. The agent of claim 2, wherein the polymer is PEG.

4. The agent of claim 2, wherein the polymer is PDMA.

5. The agent of claim 2, wherein the polymer is POEGMA.

6. The agent of claim 5, wherein the POEGMA is a polymer of POEGMA300 or POEGMA500.

7. The agent of claim 1, wherein the galectin 3C comprises a sequence of SEQ ID NO:11, or a variant thereof comprising at least 90%, 92%, 95%, or 98% sequence identity therewith.

8. The agent of claim 7, wherein the at least one substituted cysteine residue replaces threonine at position 243 (T243C).

9. The agent of claim 1, wherein the polymer and sequence are linked by a succinimide molecule or similar thioether linkage resulting from a reaction between a thiol and maleimide.

10. A method for treating a subject with cancer comprising administering a therapeutically effective amount of a composition comprising the agent of any of claim 1.

11. The method of claim 10, wherein administering comprises intravenous administration.

12. The method of claim 10, wherein the cancer is a hematological cancer.

13. The method of claim 12, wherein the cancer is multiple myeloma.

14. The method of claim 10, wherein the cancer is ovarian cancer.

15. A method of improving the pharmacokinetics or thermal stability of a protein drug compound comprising:
a) obtaining a sequence comprising SEQ ID NO: 11 or variant thereof comprising at least 90%, 92%, 95% or 98% sequence identity therewith, wherein the Gal3C sequence or variant thereof comprises at least one substituted cysteine residue in place of a rationally-selected and/or solvent accessible residue; and
b) conjugating a polymer to the Gal3C sequence or variant thereof to the at least one substituted cysteine residue utilizing a thiol-Michael reaction.

16. The method of claim 15, wherein the conjugating step comprises combining a polymer comprising a maleimide molecule covalently bound thereto, wherein the maleimide molecule reacts with a thiol group of the at least one substituted cysteine residue resulting in the polymer being linked to a sulfur of the at least one substituted cysteine residue via a succinimide molecule.

17. The method of claim 15, wherein the polymer covalently bound to a maleimide molecule comprises a polymer based on monomers of the following: [chemical structure]


SEQUENCE LISTING (13 sequences)

SEQ ID NO: 1 - DNA, 25 bp, synthetic construct
  source 1..25: mol_type = other DNA
cccctccatggctgggccactgatt

SEQ ID NO: 2 - DNA, 25 bp, synthetic construct
  source 1..25: mol_type = other DNA
ccagccatggaggggcgccataggg

SEQ ID NO: 3 - DNA, 25 bp, synthetic construct
  source 1..25: mol_type = other DNA
agacctctgcagtgcttcatatacc

SEQ ID NO: 4 - DNA, 23 bp, synthetic construct
  source 1..23: mol_type = other DNA
agcactgcagaggtctatgtcac

SEQ ID NO: 5 - DNA, 51 bp, synthetic construct
  source 1..51: mol_type = other DNA
ctgataacaattctgggcacggtgattcccaatgcaaacagaattgcttta

SEQ ID NO: 6 - DNA, 51 bp, synthetic construct
  source 1..51: mol_type = other DNA
taaagcaattctgtttgcattgggaatcaccgtgcccagaattgttatcag

SEQ ID NO: 7 - DNA, 51 bp, synthetic construct
  source 1..51: mol_type = other DNA
tcggttttcccatttgaaagtgggattccattcaaaatacaagtactggtt

SEQ ID NO: 8 - DNA, 51 bp, synthetic construct
  source 1..51: mol_type = other DNA
aaccagtacttgtattttgaatggaatcccactttcaaatgggaaaaccga

SEQ ID NO: 9 - DNA, 51 bp, synthetic construct
  source 1..51: mol_type = other DNA
ccatttgaaagtgggattccattcattatacaagtactggttgaacctgac

SEQ ID NO: 10 - DNA, 51 bp, synthetic construct
  source 1..51: mol_type = other DNA
gtcaggttcaaccagtacttgtataatgaatggaatcccactttcaaatgg

SEQ ID NO: 11 - protein, 149 aa, synthetic construct
MAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYCMILEHHHHHH

SEQ ID NO: 12 - protein, 250 aa, Homo sapiens [sequence omitted]

SEQ ID NO: 13 - protein, 149 aa, synthetic construct
MAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMILEHHHHHH